SEQUENCE LISTING (51 sequences)

SEQ ID NO: 1 - protein, 799 aa, Triticum aestivum
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGVRDTVPPFDPFNHSGLGWTFDRAEAHKLIEALGHCLRTYRDYKESWRGLQERGMSQDFSWEHAAKLYEDVLLKAKYQW

SEQ ID NO: 2 - protein, 798 aa, Triticum aestivum
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 3 - protein, 799 aa, Triticum aestivum
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAAKLYEDVLVKAKYQW

SEQ ID NO: 4 - DNA, 2821 bp, Triticum aestivum
gctgccaccacctccgcctgcgccgcgctctgggcggaggaccaacccgcgcatcgtaccatcgcccgccccgatcccggccgccgccatgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctcaagtcggacggctacaccaacttctccctggggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggttgaaccagctttacgccatggcctacggcaccgtccccgtcgtgcacgccgtcggcggggtgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtgaacgctagctgctagccgctccagccccgcatgcgtgcatgcatgagagggtggaactgcgcattgcgcccgcaggaacgtgccatccttctcgatgggagcgccggcatccgcgaggtgcagtgacatgagaggtgtgtgtggttgagacgctgattccgatctcgatctggtccgtagcagagtagagcggacgtagggaagcgctccttgttgcaggtatatgggaatgttgtcaacttggtattgtagtttgctatgttgtatgcgttattacaatgttgttacttattcttgttaagtcggaggcaaagggcgaaagctagctcacatg

SEQ ID NO: 5 - DNA, 2793 bp, Triticum aestivum
gcactccagtccagtccagcccactgccgcgctactccccactcccactgccaccacctccgcctgcgccgcgctctgggcggaccaacccgcgcatcgtatcacgatcacccaccccgatcccggccgccgccatgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtgaacgctagctgctagccggtccagccccgcatgcgtgcatgacaggatggaattgcgcattgcgcacgcaggaatgtgccatggagcgccggcatccgcgaagtacagtgacatgaggtgtgtgtggttgagacgctgattccgatctggtccgtagcagagtagagcggaggtagggaagcgctccttgttacaggtatatgggaatgttgttaacttggtattgtaatttgttatgttgtgtgcattattacaaagggcaa

SEQ ID NO: 6 - DNA, 2846 bp, Triticum aestivum
ggagaatagccacatccctggtttggagagaccatcgcagcaacaaccattaccaccacaacaaacattatcgcaccaacaaccacaacaacccatccagtccagcccactgccaccgcgctactctccactcccactgccaccacctccgcctgcgccgcgctctgggcggaccaacccgcgaaccgtaccatctcccgccccgatccatgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgaggagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtgaacgctagctgctagccgctccagccccgcatgcgtgcatgacaggatggaactgcattgcgcacgcaggaaagtgccatggagcgccggcatccgcgaagtacagtgacatgaggtgtgtgtggttgagacgctgattccaatccggcccgtagcagagtagagcggaggtatatgggaatcttaacttggtattgtaatttgttatgttgtgtgcattattacaatgttgttactt

SEQ ID NO: 7 - DNA, 6898 bp, Triticum aestivum
gggggccgttcgtacgtacccgcccctcgtgtaaagccgccgccgtcgtcgccgtcccccgctcgcggccatttccccggcctgaccccgtgcgtttaccccacagagcacactccagtccagtccagcccactgccgccgcgctactccccactcccgctgccaccacctccgcctgcgccgcgctctgggcggaggaccaacccgcgcatcgtaccatcgcccgccccgatcccggccgccgccatgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtagttggttcgttatgacttgctgtatggcgcgtgcgcctcgagatcagctcacgaattgtttctacaaaacgcacgcgctcgtgtgcaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggcatggacattacctcttcagtctctcttcccgttgttcataaaactttgctcgaatcactcataagaacaaacattgtgttgcataggtggtcttggagatgttgcgggtgctctgcccaaggctttggcaaagagaggacatcgtgttatggtactgcaggctttcacttaactctgttgagtccatatgttcgaataatatcagtgattggcataatgttattaagtgcaagacatgaaagtgttcttctgttagagtatttcatagccaaccctggaggttaggttgttggggcctactgggtgcgggagggggtttgcaaaaagtggtggttagcagtcggatttcacaaataaggaggctgataaccacgccatcagtgaagggaatgagtgtcgggtacccgatcgaccgttttgcccgacgtcaggtttacctgccctgtagatccgaataagtagttcctatcctcagttaagtaccaaatatcgccagcacccgtgtgtgtatttatagtactggatgatcaatttatcaacatttccggttaatggttgctatcatattcactgtaattgttagtaaacagtggatgtttgtaatgtagatgatggctaaatgtatgttgtcaagctttcattttaaagaaaattttattgggagctagttttcgggtttggttagagccaccaaaaccccagaatttttgggagttggcttgtgacagagggttttggggagttaactttcgggattcagttagagacgctcttactagttccagtaaagagtaaactattttctgcagtcatcccaattgttctgtagaaattaaaagtagaaaatagttgtggtatcatataaaccatatattattcaaaatctagaatcatggacttggctagactttgatgatctgaaattttaaatttgatgataattgagaaatgatcctttctatcttaggttgtggtaccaaggtatggggactatgaggaagcctacgatgtcggagtccgaaaatactacaaggctgctggacaggtaagcgaaaatgcaatcaaaggggagctgaaatttcaatgcttactatcataataaatcaattttaagtaaaaaaatttgtcctgcaggatatggaagtgaattatttccatgcttatatcgatggagttgattttgtgttcattgacgctcctatcttccgacaccgtcaggaagacatttatgggggcagcagacaggttaattttctatatgttggtgtttgattgcactgataaactgagaataagccaaggcctactgactggcatatgattacacattttattttttcaggaaattatgaagcgcatgattttgttctgcaaggccgctgtcgaggtatcctctccaactcaattgacaacctattaccactatacaattatgtgtatgcatgtatttcaacagatacgtaatctcccttgtgaagtgtatatatactaataacatttcaatacctcacatgcacatttggtcaagcgttatgatttaacttctgataatctattgcactgatgaacaataatattgatgatccttgttacttcatcgttatgtttatgttctcttcaccggcgcattgattttggaaatagcatttccacctgccacaaacaataatatatactcctactttcatccaatgtagatattttcgcacttggcatatcatcccattaaatattattggtccatcatttttattcctctataatttgcaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccaggttccttttctcctaatcttgttttttctctagtctctactattcactccacattgtttgaggaaactaaaggggttgcaaaattatgatggcttatgaaagttatggaggtaaatgcatcagtggtgcttgaacttgtcacgcatgttcactttggtgcttacagttgtagactacggaaaactggtgcaaaaacttggctattgtgtgcaatacggtgtattttccgtatgtagggtcaaatgttgcctatgtggcattgtattcccgtctatagatgttagaccgtgcctacatcgccattgggcccacacactccctattacatgtgggacccacttgtcagcctatgacataaataaaatggaaatttataataaaaatgatggcctggggtcttgaaaatgggacctcgcaggtatgccgctagccagcacgccctaatcattaatcccctatgcacttcagtatgtgtgtgtctgtgtgtggagtcggggggggggggggtatgtattcttatatcctttgctctaaggctatcattggcgtgctagcaccgccgggtctccatcaacaccgacatcatccacgaccccatccagctcttcctcaacaagcccacctccagcctcaactcgggcagcaccttcatggtggcctaggtgctctgcaccatcgctcgaagtggcaacgtcgttgtcatgaccatccaccaacccaacacgcaaaatcctcaacatcatttgacagtgagcatgcccctcttgtcatttccccctcatacccaaacctgtctcgataacccttggagctgcacaagttgtgaccatcgcctgcgtcgctgcacaacgcctgacctagccggaccattatagaagcctgccttgggagcccatacctccctgcacatcctcctctttccccatagaccgtgccgccatcgcaaatcgacttctcctctcctccttctcctgctctggccgttttccccgccgcgaagctgcaatccatggcgagttggccatggccctattccccaattgctcgcactaggaggtcctccttgaagcctagcacctttccccctcactaattgcaagttggggagcccctcacgagctccctacattggccgtagtcgcctgccgcctcaactctggtccagacctcgttcccgtggcctcgacgacatctcctcgacctcccattccacacgcggcctggagaggatcaccgcatgttcatccatccgacccgaatcatcatagaaccaacgccagagaggtcatcccgacgacgtcgcactgttcctctatttcccccaagctgtgtcgcgtcataatataagacgggcttgtttgtatctctaggggtcatcgggttcaatggctagctcatgcatggacctgactttaggtcccaggttcgaacccccgcgtgcacataatttatttgctatttatttctcctatctactaataagtgggacccacacatcatactaagccctttttgtctcttgcctgctgataagtgggacccacacgcaatacttagccagagagagaacatgagcttgttggtgccgcgttggcaagccacgccagcagtcttaacggctacaaacagaggatatggtgtcacatcaacgtgcagagcgtttacgaatggaaagtgtactatatgcacacaagagccagagccaggtttttgcaccagttttttgtattctacaactgcgagcaccaaagtgtacatgccgaaccaaagtgaacacggcgagtccattcttttctggtacgatgggtggctcaaagacaccccaatagaagctatcacctcggacattgccaattgggtgccgaactacattaaagtggcaaggtcagttgattgcgctatgtgttggatcagggacataaacgattccataaatattgcaatgttcattcaaattcttaacatttgcgaggcgcttcatgatttccatctccctcatatcagagacatttggtcgtgtacactaaatttctcaggtcacttctcgtctaaatccgcatatgtagctcacttcaatgacttgactttggtccagctaacgccatttggcgctcttgggcccctttgcgtagcaattttttcatatggctcgctccgcgcaataggatttggatcacgggcagacgcgctagatgaggtcttccacacaatgaacattgcgttctttgctctgctcttccagaagacacttgtgattttattacgagttgtgccatagatgccggtgggtttcagctaggaacagggtgtcaccttcggacaagaagaagttgcatagtttggtcgtcttaactgcttggtcgatttggaaggagcacaacaacagtctttgaaggcaaagctaattccctcgatcaagttattagacggatcaaatgtggtacagtgccatggctagttgcttggagtcacttttaagctaggtcgcttgccatcacgctttgtgttaagcgcttggggtcgcttttgctcaatttgtattttgttgttatgtgtttttagttatgtagcctgaactttctggacttagttttttcctctataatgatcacatgctttggtcagttattcctttcttgggtactccgttgggctaattctttctcttgattgatgttgtatatgcagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 8 - DNA, 6811 bp, Triticum aestivum
gggggccgttcgtacgtacccacccctcgtgtaaagccgccgccgtcgtcgccgtcccccgctcgcggccatttcctcggcctgaccccgtgcgtttaccccacacagagcacactccagtccagtccagcccactgccgcgctactccccactcccactgccaccacctccgcctgcgccgcgctctgggcggaccaacccgcgcatcgtatcacgatcacccaccccgatcccggccgccgccatgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggtagttagttatgaccaagttatgacgcgtgcgcgcgccttgagatcatcgtcgtctcgctgacaaattgtttatacaaaacgcacgcgcgcgcgtgtgtgcaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggcatggacattacctcttcagtgtcttttttctctctgttcataaaacctggcttgaattactcataagaacaaacgttgtgttgcataggtggtcttggagatgttgccggtgctttgcccaaggctttggcgaagagaggacatcgtgttatggtaccacatgctttcatttaactctgttgaatccatatgttcgaataatatcagtgagcagtataatgttattaagtgcaagacatgaaagtgttcttctgttatagagtatttcatagccaaccctggaggttaggttgttggggcctactgggtgtgggagggggtttgaaaaaagtgttggttagcagtcggatttcacaaagaacgctgataaccacgccatcagtgaagggaatgaatgtcgggtacccgatcgaccgttttgcccgacgtcaggtttacccgccctgtaggtccgaataagtagttcctatcctcagttaagtaccaaatatcggcagcgcccgtgtgtgtatttatagtactggatgatcaatttatcaacattttcagttaatggttgctatcatattcactgtaattgttagtaaacagtggatgtttgtaatgtagatgatggctaaatgtatgttgtcaagctttcatttcaatgcaatttttattgggagctagttttggggttcggttagagccaccaaaaccccagaatttctgggagttggcttgtgagagagggttttggcgagttgactttcgggattcagttagagacgctcttactagttccagtaaagaagtaaactattttctgcagacatcccaattattctgtagaaattagaagtagaaaatagttatggtatcatataaccatatattattcaaaatctagaatcatggacttggctagactttgatgatctgaaattttaaatttgatgataattgagaaatgatcctttctatcttaggttgtggtaccaaggtatggggactatgaggaagcctacgatgtcggagtccgaaaatactacaaggctgctggacaggtaagcgaaaatgcaatcaaaggggagctgaaatttcaatgcttactatcataataaatcaattttaagtgtttttttttgtcctgcaggatatggaagtgaattatttccatgcttatatcgatggagttgattttgtgttcattgacgctcctctcttccgacaccgccaggaagacatttatgggggcagcagacaggttaatcttctatatgttggtgtttgattgcactgataaactgagaacaagccaaggcctactgaccggcatatgattacacattttattttttcaggaaattatgaagcgcatgattttgttctgcaaggccgctgtcgaggtatcctctccaactcaattgacaacctattacaactatacaattatgtgtatgcatgtatttcaacagatacgtaatctcttgtgaagtgcatatatactaacaacatttcaataccttacatgcacatttggtcaagcgttatgatttaacttctaataatctattgcactgatgaacaattatcttgatgatccttggtacttcatcgttatgtttccatgttctcttcaccgattgatttggaaatagcatttccacctgccacaaacaataatatacactcctactttcatccaatgtagatattttcgcacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctccaaatcttgatttttctctagtctctactatttactccacattgtttgaggaaactaaacgggttgcaaaattatgatggcttatgaaagttatagtcttatataggtaaatgcaccagtggtgcttgcacttgtcacgcgtgttcactttggtgcttacagttgtagacaataaaaaactagtgcaaaaacttggctgttgtgtgcaatacggtgcattttccgtatgtaggagtcaaatattgcctgtgtggcattgtattcccgtctatagctgttagaccatgcctacgtcgccattgggcccacacaccctctattacatgtgggccccacttgtcagcctatgacataaataaatggaaatttataatgaaaatgatggcctggggtcttgaaaatgggccgtcgcaggtatgctggtagccagcatgccctaatcattaatccccctatgcacttcatgtcttgtgtatgtgtgtgtgggaaagggggggggtatgtatgcttatgcttatatcctttgctccaaggctgccatcctcaacaagcccacctccagcttcaacacggccagcgccttcatgatggcccaggtgctccgcaccatcgatcgaagcggcaacgtcgtcgtcacgaccatccaccaacccaacgcacaaaatcctcaggctccgttcggtacggaggaagagaaaatgcaggaaaaaacaacttcatgggaacaaggtttggtgaacagtaaaaacatgtggaatctgaaaatgtaggtaccagaaaaaccggcctgttcggtttgcaggaaaaagcatacttgcagcagcactgtttggccctttccagtgtaaggctaaccatagtgggagtaacataactaatattatgtacttggaactcacaaacatgcttatgtggcaggcaattaaagaagagagagagagtcatagtaacatagttagataccgtatcataataaatattatgttactatgtgtcatgcatgacaataaatgagaccatctgtgatactacgttatgatattatgcactatagatgtagtatcatacactagtatcatatgcatgatactagtgtatgttactccccactatgaccagcctaacgaggggatgggcatcagtagtgattaccagtttttattattttttattcggctggacgccctactcttgtggtgcgtagcggaaaaaggcagtgctagcttcgggactccgcgagcacatgaggttctaccggattttagatttcctataaaaagtacaggctcacgcaacttttcaatggaacagaccatcaagattcctttgaaccgaacgcactgcatgcaagaattccaatgaaaaacgagccgtcaaatgttcctgcgaatttcctctataccgaacagaccctcaacatcctcaaatagtgagcatgcccctcttgtcctttccccctcgtacccaaacgccatttggcgctcttggtgttggatcatttgcctcgggcatttgccaatttggcgccgaaccatattaaagctatgactacttggtgttggatcagggacgcaaagaatcccataaatattgcaacgttcattcaaattcttaacatttgcgaggcgcttcatgatttccatctccgtcaggtctgagacatttggtcgtgtacactaaatttctcaggtcacttctcgtctaaatccgcatatgtagctcacttcaatgacttgcctttggtctagctaacgccatttggcgctcttgggcccccttgcttagcaaatttttcatatggctcgcactgcgcaagaggatttagatcacgggcagacgcgctagacgaggtcttccgcacaatgaacattgcgttctttgctctgctcttcccggagacacttgtgatcttattacgagttgtgccatttcaaacatctgtctctccatggtcgctccagccatagatgccttgttctctgaatggtgggtttcagctaggaacagggtgccaccttcgacaagaagttgcatagtttggtcgtcttgactgcttggtcgatttggaaggaacgcaacataagagtctttgaaggcaaagctaatttctttgatcaagttattagccagatcaaatgtgatgtattctactggtacaaggccggggctatttgctttgagtcactttttagctaaggccgcttgggctaagcgcttggggtcgtttttgctcaactgtagcctgaactttctggactttgtaattttttttatcctctataatgatcacatacagctctcctgcatggttcgaaaaggaaaaatgtgaacatgtgtggcaagtttaagcacaacccgtgcatttacctcaaagttatacaacactgacatgctgaattacattttttttgaggatctgacatgccgaattacatgctttggacagttattcatttcttcggtacaccattggctaattatttctcttgacagttgctgaattagtacatgctttggtcgcagttattcctttgttcggtactctgttgggctaattatttctcttgattgatgttgcatgcagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 9 - DNA, 6950 bp, Triticum aestivum
gggggccgttcgtacgtacccgcccctcgtgtaaagccgccgccgtcgtcgccgtcccccgctcgcggccatttcttcggcctgaccccgttcgtttacccccacacagagcacactccagtccagtccagcccactgccaccgcgctactctccactcccactgccaccacctccgcctgcgccgcgctctgggcggaccaacccgcgaaccgtaccatctcccgccccgatccatgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtagttagttatgaccaagttatgacgcgtgcgcgcgcctcgagatcatcgtcgtctcgctcacgaattgtttatttatacaaaacgcacgcccgcgtgtgcaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggcatggacattacctcttcagtctctcttcctgttgttcataaaactttgctcgaattactcataagaacaaacattgtgttgcataggtggtctgggagatgttgcgggtgctctgcccaaggctttggcaaagagaggacatcgtgttatggtactacaagctttcatttaactctgttgggtccatatgttcgaataatatcagtgagtagtataatgttattaagtgcaagacatgaaagtgttcttttgtcatactccctccgtaaattaatataagagcgtttagattactactttagtgatctaaacgctcttatagtagtttacagacggagtagagtatttcatagccaaccctggaggttaggttgctgaggcctactgggtgggggagggggtttgaaacaagtggtggttagcagccagatttcacaaagaaggaggctgataaccacaccatcagtgaaggaatgaatgtcgggtacccgatcgaccgttttgcccaacgtcgggtttacccgccctatagatccgaataagtagttcctatcttcaattaggtaccaaatatcgccagcgcccgtgtgtgtatttatactactggatgatcaatttatcaacatttccggttaatggtttctatcatattcactgtaattgttagtaaacagtagatgtttgtaatgtagatgatggataaatgtatgttgtcgagctttcatttcaatgcaattttgattgggagctagtttcgcggttcggttagagccatcaaaaccccagaatttttgggagttggcttgtgagagagggttttggggagttaactttcgggattcagttagagacgctcttactagttccagtaaagagtaaactattttctgcaggcatcccaattattctgtagaaattagaagtggaaaatagttatggtatcatataaaccatatattattcaaaatctagaatcatggacttggctagactttgataatctgaaattttaaatttgatgataattgagaaatgatcctttctatcttaggttgtggtaccaaggtatggggactatgaagaagcctacgatgtcggagtccgaaaatactacaaggctgctggacaggtaagcaaaaatgcaatcgaaggggagctgaaattttattgcttattgtcataataaatcaatttttaagtgttttttttgtcctgcaggatatggaagtgaattatttccatgcttatatcgatggagttgattttgtgttcattgacgctcctctcttccgacaccgtcaggaagacatttatgggggcagcagacaggttaatcttctatatgttggtgtttgattgcactgataaactgagaacaagccaaggcctactgactggcatatgattacacattttattttttcaggaaattatgaagcgcatgattttgttctgcaaggccgctgttgaggtatctctccaactcaattgacaacctattaccactatacaattatgtgtatgcatgtatttcaacagatacataatctcttgtgaagtgcatatatactaataacatttcaataccttacatgcacatttggtcaagcgttatgatttaacttctgataatctattgcactgatgaacaattatcttgatgatccttgttacttcatcgttatgtttccatgttctcttcaccgcgaattgatttggaaatagcatttccacctgccacaaacaataatatacactcctactttcatccaatttagatattttcgtacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctcctaatcttgatttttctctagtctctactatttactccacattgtttgaggaaactaaacgggttgcaaaattatgatggcttatgaaagttatagtcttatagaggtaaatgcaccagtggtgcttgaacttgtcacgcgtgttcactttggtgcttacagttgtagactatgaaaaacgggtgcaaaaacttgctgttgtgtgccatacggtgcattttccgtatgtaggagtcaaacgttgcctatgtgggcattgtattcccgtctatagctgttagaccgtgcctacgtcgccattgggcccacacactctctatttacatgtgggccccacttgtcaacctatgacataaataaatggaaatttataataaaaatgatggcctggggtcttgaaaatgggacctcgcaggtatgctggtagccagcacgccctaaacattaatcccctatgcacttcatgtcttgtgtatgtgtgtgtctgtgtggggagggggggggtatgtatgcttatatcctttgctccaaggctaccatcctcaacaagcccacctccgcttcaacacggccagcgccttcatgatggcccaggtgctccgcaccatcgctcaaagcggcaacgtcgttgtcatgaccatccaccaacccaacacacaaaatcctcaacatccgcaaatagtgagcatgcccctcttgtcctttcccctcgtacccaaacatgtcttgataacccttggagctgcacaagttgtgaccatcgcctgcgtcgcctcatagagcccgacctagccggaccgttatagaagcctacttgggagcccatacctccctgcacatcctcctctttccccatagatcgtgccgccatcgcaaaccaacttctcctctccttctcccactctggccgtttcccccgccgcgaagctgcaatacatgccgagttggccatggccctattccccaattgctcgcactaggaggtcctcctctaagcctagcaccttttcccctcaccaattgcaagttggggagcccctcgcgagctccctacgtcggctgcagttgcctgccgcctcaactctgatccagacctcgttcccgtggcctcggcgacatctcctcgacctcccattccacacgtggcctggcgaggatcaccgcatgttcatccatgtgaaccgaatcatcatagaactaacaccggagaggtcatcccgacggcgtcgcactgttcctctattccccccaagccgtgtcgcgtcataatataagacggacttatttgtatcccttgggtcatcggttcaatggctatttctttctcctgtctactgataagtgggacccacacgccacactaagccctttctttctcctacccgttgataagtgggacccacacacagtacttagccagagagagaacatgagcttgttggtgccacgtcggcaagccatgtcagcagtcttaacggctacaaacaacggatatggtgtcacgtgagcgtttacgaatggaaagtgcatcatactgcatgcgagagccagagccaggtttttgcaccagttttctgtattttacaactgcgagcatcaaagtgtacatatgccgaaccaaagtgaacatggtgagtccattcttttctggtgcggtgggtggctcaaagacaccccaatagaagctattgcctccgacattgccaattcggtgccgaaccatattgaagtggtgaggtcagttgcttgtgctatgactactaggtattggatgagggacataaaggatctcataaatattgcaatgttcattcaaattcttaacatttgcgaagcgcttcatgatttccatctcccctagatcagagacacttggtcgtgtacactgaatttctcaggtcgcttctcgtctaaatccgcatatgtagctcacttcaatgacttgcctttggtccagctaacgccatttgcgtagcaaatttttcatatggctcgctctgcgcaagaggatttggatcacgggcagacgcgctagacaaggtcttccgcacaatgaacattgagttttttgatccgctcttcccgaagacacttgtgatcttattacgagttgtgccatttcaaacatctgtctctccatggtcgccccagccatagatgccttgttctctgaatggtgggtttcagctaggaacagggtgccaccttcggacaagaagttgcgtagtttggtcgtcttaactgcttggttgatttggaaggaacacaacaacagtctttgaaggcaaagctaattccttcgatcaagttattagacggatcaagtgtgatgaatcctactggtacaatgccgttgctagttgcttggagtcactatttggctaggtcgcttgccatcccgctctgtgctaagcgcttggggtcgcttttgctcaatttgtattttgttgttatgtgtttttagtaatgtaacctgaactttctggactaagtagaaaaaaattctcctccataatgatcacatacagttctcctgcatggttcgaaaaaaaaatgagaacatccgtggcaagtttaagcaccaccggtgcatttttacctcaaagttatatacaacactgacatgccgaattacatgctttggtcagttattccattcttcggtactccgttgggctaattctttctcttcatgttgcatgcagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagc

SEQ ID NO: 10 - protein, 676 aa, Triticum aestivum
MLGHIASSSAAFLLLVASSSSSRRWRSSSPRSFGAAGTRLHWERRGFSRDGSVPCRRVSAAAAVAGGEEGAGKAIAGEGSRQAASGQRGKLEAAENEDAVSQKSVASAPRPDSTVAEQHWAAGSRSEVDPSAPVSAAASGYWKDVVVAEQVGAKVDAGGDAAKVWSSPVDSENMGSAPLAGPNVMNVIVVASECAPFCKTGGLGDVVGALPKALARRGHRVMVVIPKYGDYGEARDLGVRKRYKVAGQDSEVSYFHSYIDGVDFVFLEAPPFRHRHNDIYGGERPDVLKRMILFCKAAVEVPWYAPCGGTIYGDGNLVFIANDWHTALLPVYLKAYYRDNGLMQYTRSVLVIHNIAHQGRGPVDDFNIMDLPEHYMDHFKLYDPLGGQHNNVFAAGLKMADRVVTVSHGYMWELKTMEGGWGLHDIINQNDWKLDGIVNGIDTAEWNPAVDVHLHSDDYTNYTRDTLDIGKRQCKAALQRELGLQVRDDVPLIGFIGRLDHQKGVDIIAAAMPWIAQQDVQLVMLGTGRPDLEDMLRRFEGEHRDKVRGWVGFSVRMAHRITAGADVLLMPSLFEPCGLNQLYAMAYGTVPVVHAVGGLRDTVAPFDPFGGTGLGWTFDRADAGRMIDALGHCLNTYWNYKESWRGLQVRGMSQDLSWDRAAELYENVLVKAKYQW

SEQ ID NO: 11 - protein, 674 aa, Triticum aestivum
MSGAIASSSAAFLLLVASSSCSSSSPRRWRSSSPRSFAAAGTRLHWERRGFSRDGPVPCRSPCAAAGGEDGAAAGESSKEGVAGLRGKLKAAENEDPVSQKSVASAPRLDSSVAEQNGAAATRSKADPSAPVSAAASGYWKDVVVAEQVGTKVDTGGDAAEVSRSPVDSENKGSAPLAGPNVMNVIVVASECAPFCKTGGLGDVVGALPKALARRGHRVMVVIPKYGDYAEARDLGVRKRYKVAGQDSEVSYFHSYIDGVDFVFLEAPPFRHRHNDIYGGERPDVLKRMILFCKAAVEVPRYALCGGTIYGDGNLVFIANDWHTALLPVYLKAYYRDNGLMQYTRSVLVIHNIAHQGRGPVDDFNIMDLPDHYMGHFKLHDPLGGEHNNVFAAGLKMADRVVTVSHGYMWELKTVEGGWGLHDIINQNDWKLDGIVNGIDTAEWNPAVDVHLHSDDYTNYTRDTLDIGKRQCKAALQRELGLQVRDDVPLIGFIGRLDHQKGVDIIAAAMPWIAQQDVQLVMLGTGRPDLEDMLRRFEGEHRDKVRGWVGFSVRMAHRITAGADVLLMPSLFEPCGLNQLYAVAYGTVPVVHAVGGLRDTVAPFDPFGGTGLGWTFDRADAGRMIDALGHCLNTYWNYKESWRGLQVRGMSQDLSWDHAAELYENVLVKAKYQW

SEQ ID NO: 12 - DNA, 2727 bp, Triticum aestivum
acccgtcgtcttcctcggcgcgggcgctctagctcttcccagtctcggcctccgagctccaccacacaagccgctcgcctgtccccggcctccagttcctccgcctcccgcccccccccccccccccgctccgtaccgccgcaaaaaacctgcagccacacgtagccagcgcagatctttcttccacccgcgcttaatttccctcgtgctcgcgccgctcgccgtcgccctcgtccactactactctgccttgtttgtccggctgtttctgttcatccccgaagacttctggcgcgtcggatcccgggatgttggggcacatcgcttcctcgtccgcggcgttcctcttgctcgtggcctcctcctcgtcgtcgcggcgctggcggagcagctcaccgcgctctttcggtgccgccggtacgcggctgcattgggaacggcgagggttttctcgggacggatcggtgccgtgccgccgtgtttcagcagcagcggcagtggctggtggtgaggagggcgcggggaaggcaatagcaggggagggctcgaggcaggcagcctctgggcagcgcggcaagctcgaggctgctgaaaatgaggatgctgtttcacaaaaatcagttgcatctgcacccaggccagatagtaccgttgcagagcaacattgggcagctgggagcagatccgaagtcgatccgtcagctcctgtctcggcggcggcatcaggttattggaaagacgtcgttgtcgctgaacaggttggggctaaggttgatgccggtggagatgcagcaaaagtgtggagttctccggttgacagtgagaacatggggtctgctcctttggctggcccgaatgtgatgaatgtcatcgttgtggcttctgaatgtgctcctttctgtaaaacaggtgggcttggagatgttgtgggtgctttgccgaaggctctggcaagaagagggcatcgtgttatggtcgtgataccaaaatatggagattacggagaagctcgtgatctaggtgttcgcaaacgttacaaggtagctggccaggattcagaagtgagctactttcattcttatatcgatggagttgattttgtgttcttagaggccccgcccttccgccaccggcacaatgatatttatggaggagaaagaccggatgtgctgaagcgcatgattttgttctgcaaggctgctgtggaggttccttggtacgctccatgcggtggtactatctacggtgatggcaatttagtcttcattgcaaacgattggcatactgcacttctacctgtttatctgaaggcgtattaccgagacaatggcctgatgcagtatactcgttctgttctcgtgatccataacattgctcatcagggacgtggccctgtagatgacttcaacatcatggacttgcctgagcactacatggatcacttcaaactgtatgatccccttggcggccagcacaacaacgtgttcgctgctggcctgaagatggcagaccgggtggtcaccgtcagccatggctacatgtgggagctgaagacgatggaaggcggctggggcctccacgacataataaaccagaacgactggaagctggacggcatcgtgaacggcatcgacacggccgagtggaaccccgcagtcgacgtgcacctgcactccgacgactacaccaactacacccgagacacgctggacatcggcaagcggcagtgcaaggcggccctgcagcgcgagctgggcctgcaggtccgcgacgacgtgccgctcatcgggttcatcgggcggctggaccaccagaagggcgtggacatcatcgcggcggcgatgccgtggatcgcgcagcaggacgtgcagctggtgatgctgggcacggggcggccggacctggaggacatgctgcggcggttcgagggggagcaccgggacaaggtgcgcgggtgggtggggttctcggtgcggatggcgcaccggatcacggcgggcgcggacgtgctcctcatgccgtcgctgttcgagccgtgcgggctgaaccagctgtacgcgatggcgtacgggaccgtgcccgtggtgcacgccgtcggcgggctccgggacacggtggcgccgttcgacccgttcggcggcaccgggctggggtggacgttcgaccgcgcggacgccggcaggatgatcgacgcgctcgggcactgcctcaacacgtactggaactacaaggagagctggaggggcctgcaggtccgcggcatgtcgcaggacctcagctgggaccgcgccgccgagctctacgagaacgtcctcgtcaaggccaagtaccagtggtgatgcggctgatgctgacccctctactgcaacgcatgcacgtacttaggggacagccggaagtggtgcccacgatcgcgatcactggcgccccggagtagccaagtgtcacgcgatgccaccggcggcagccggacgacggtggaggtagtgaaccgtgcccgtggcgccgcccaacttttgggttcacgagttgtacacaccagttcagtcaggttcgtggcttctgtgaattaccgagtcgtaggcgatcaaataggaagttgcagagtttctatactgccaattttctcagctcttggtgtggaaaaacacagttctgagaaggttcatcactggacgctgaccacgttcacgttctgtttcatgctcaaaaaaaaaaaaaaaacga

SEQ ID NO: 13 - DNA, 1282 bp, Triticum aestivum
tggtgaggatggcgcggcggcaggggagagctcgaaggagggggtcgctgggctgcgcggcaagctcaaggttggtaacgctatcattccttcttctgatggattccatttcgtgctcaggctgctgaaaatgaggatccggtttcacaaaaatcagttgcatctgcacccaggctagacagtagcgttgcagagcaaaatggggcagctgcgaccagatccaaagccgatccgtcagctcctgtctcggcggcagcatcaggttattggaaagacgtcgttgtcgctgaacaggttggaactaaggttgacaccggtggagatgcagcagaagtgtcgagatctcccgttgacagtgagaacaaggggtctgctcctttggctggcccgaatgtgatgaatgtcatcgttgtggcttctgagtgtgctcctttctgtaaaacaggtgcgcatacataaactgagcaatttgaccaataatgatatcatgcataggcgggcttggagatgtcgtgggtgctttgcccaaggctctggccagaagagggcatcgcgttatggtacccactcctttgcttttctcttatacaagtatttctttcaattttaggttgtgataccaaagtatggcgattacgcagaagctcgtgatcttggtgttcgcaaacgttacaaggtagctggccaggtatgttaaataacgagtctgcagtaattgaattgtgttctggtttgcaggattcagaagtgagttactttcactcttatatcgatggagttgattttgtattcttagaggccccgcccttccgccaccggcacaatgatatttatggaggagaaagaccggtaagcgtctgtttccctagagctttgtacatttccatcatttttggcaggatgtgctgaagcgcatgattttgttctgcaaggctgctgtcgaggtaccttcacaaattgtattgctattgttcatctgttcgagcatttgtaggttccttggtacgctccatgtggtggtactatctacggagatggcaatttggtcttcattgcaaacgattggcatactgcacttctacctgtttatctaaaggcatattaccgagacaatggcctgatgcagtatactcgttctgttctcgtgatccataacattgctcatcaggttttacacctcaatctttaactgctggacactaaaattttgctttgcagggacgtggccctgtagatgacttcaacatcatggacttgcctgaccactacatgggtcacttca

SEQ ID NO: 14 - DNA, 2025 bp, Triticum aestivum
atgtcgggggcaatagcttcctcgtccgcggcgttcctcttgctcgtggcctcctcctcctgctcctcctcgtcgccgcggcgctggcggagcagctcaccgcgctctttcgctgccgccggtacgcggctgcattgggaacggcgaggattctctcgggacggaccggtgccgtgccggagcccctgcgcagcagctggtggtgaggatggcgcggcggcaggggagagctcgaaggagggggtcgctgggctgcgcggcaagctcaaggctgctgaaaatgaggatccggtttcacaaaaatcagttgcatctgcacccaggctagacagtagcgttgcagagcaaaatggggcagctgcgaccagatccaaagccgatccgtcagctcctgtctcggcggcagcatcaggttattggaaagacgtcgttgtcgctgaacaggttggaactaaggttgacaccggtggagatgcagcagaagtgtcgagatctcccgttgacagtgagaacaaggggtctgctcctttggctggcccgaatgtgatgaatgtcatcgttgtggcttctgagtgtgctcctttctgtaaaacaggcgggcttggagatgtcgtgggtgctttgcccaaggctctggccagaagagggcatcgcgttatggttgtgataccaaagtatggcgattacgcagaagctcgtgatcttggtgttcgcaaacgttacaaggtagctggccaggattcagaagtgagttactttcactcttatatcgatggagttgattttgtattcttagaggccccgcccttccgccaccggcacaatgatatttatggaggagaaagaccggatgtgctgaagcgcatgattttgttctgcaaggctgctgtcgaggttcctaggtacgctctatgtggtggtactatctacggagatggcaatttggtcttcattgcaaacgattggcatactgcacttctacctgtttatctaaaggcatattaccgagacaatggcctgatgcagtatactcgttctgttctcgtgatccataacattgctcatcagggacgtggccctgtagatgacttcaacatcatggacttgcctgaccactacatgggtcacttcaaactgcatgacccccttggtggcgagcacaacaacgtgttcgccgctggcctgaagatggcagaccgggtggtcaccgtcagccatggctacatgtgggagctgaagacggtggaaggcggctggggcctccacgacataataaaccagaacgactggaaactggacggcatcgtgaacggcatcgacacggccgagtggaaccccgcggtcgacgtgcacctgcactccgacgactacaccaactacacccgagacacgctggacatcggcaagcggcagtgcaaggcggccctgcagcgcgagctgggcctgcaggtccgcgacgacgtgccgctcatcgggttcatcgggcggctggaccaccagaagggcgtggacatcatcgcggcggcgatgccgtggatcgcgcagcaggacgtgcagctggtgatgctgggcacggggcggccggacctggaggacatgctgcggcggttcgagggggagcaccgggacaaggtgcgcgggtgggtggggttctcggtgcggatggcgcaccggatcacggcgggcgcggacgtcctgctgatgccgtcgctgttcgagccgtgcgggctgaaccagctgtacgcggtggcgtacgggaccgtgcccgtggtgcacgccgtcggcgggctccgggacacggtggcgccgttcgacccgttcggcggcaccgggctggggtggacgttcgaccgcgcggacgccggcaggatgatcgacgcgctcgggcactgcctcaacacgtactggaactacaaggagagctggaggggcctccaggtccgcggcatgtcgcaggacctcagctgggaccacgccgccgagctctacgagaacgtcctcgtcaaggccaagtaccagtggtga

SEQ ID NO: 15 - DNA, 20 bp, Artificial Sequence
  Synthetic
tgcgtttaccccacagagca

SEQ ID NO: 16 - DNA, 22 bp, Artificial Sequence
  Synthetic
tgccaaaggtccggaatcatgg

SEQ ID NO: 17 - DNA, 18 bp, Artificial Sequence
  Synthetic
gcggaccaggttgtcgtc

SEQ ID NO: 18 - DNA, 22 bp, Artificial Sequence
  Synthetic
ctggctcacgatccagggcatc

SEQ ID NO: 19 - DNA, 24 bp, Artificial Sequence
  Synthetic
gtaccaaggtatggggactatgaa

SEQ ID NO: 20 - DNA, 22 bp, Artificial Sequence
  Synthetic
gttggagagatacctcaacagc

SEQ ID NO: 21 - DNA, 23 bp, Artificial Sequence
  Synthetic
aaggtctccaccgtgtctcgaag

SEQ ID NO: 22 - DNA, 23 bp, Artificial Sequence
  Synthetic
gacgtacagtttgtcatgcttgg

SEQ ID NO: 23 - DNA, 23 bp, Artificial Sequence
  Synthetic
ctgctggacaggatatggaagtg

SEQ ID NO: 24 - DNA, 23 bp, Artificial Sequence
  Synthetic
gtatcaccataacggagcgactg

SEQ ID NO: 25 - DNA, 25 bp, Artificial Sequence
  Synthetic
caatcactgatggtgtaaccaaagg

SEQ ID NO: 26 - DNA, 23 bp, Artificial Sequence
  Synthetic
ccttcatgttggtcaatagcagc

SEQ ID NO: 27 - DNA, 25 bp, Artificial Sequence
  Synthetic
cagaatggacaaagaatcatccacg

SEQ ID NO: 28 - DNA, 24 bp, Artificial Sequence
  Synthetic
gaaaatacatccatgcctccatcg

SEQ ID NO: 29 - DNA, 21 bp, Artificial Sequence
  Synthetic
agggtacagggtgggcgttct

SEQ ID NO: 30 - DNA, 20 bp, Artificial Sequence
  Synthetic
gtagggttggtccacgaagg

SEQ ID NO: 31 - DNA, 20 bp, Artificial Sequence
  Synthetic
ttcgaccccttcaaccactc

SEQ ID NO: 32 - DNA, 20 bp, Artificial Sequence
  Synthetic
acgtcctcgtagagcttggc

SEQ ID NO: 33 - DNA, 20 bp, Artificial Sequence
  Synthetic
tgacgaatcttggaaaatgg

SEQ ID NO: 34 - DNA, 23 bp, Artificial Sequence
  Synthetic
ggcggcatttatcataactattg

SEQ ID NO: 35 - DNA, 22 bp, Artificial Sequence
  Synthetic
gtagatgcggtcgtttacttga

SEQ ID NO: 36 - DNA, 20 bp, Artificial Sequence
  Synthetic
ccagccaccttctgtttgtt

SEQ ID NO: 37 - DNA, 19 bp, Artificial Sequence
  Synthetic
gggccttcatggatcaacc

SEQ ID NO: 38 - DNA, 20 bp, Artificial Sequence
  Synthetic
ccgcttcaagcatcctcatc

SEQ ID NO: 39 - DNA, 23 bp, Artificial Sequence
  Synthetic
ctggacaggatctggaagtgaaa

SEQ ID NO: 40 - DNA, 22 bp, Artificial Sequence
  Synthetic
ggaacctcaacagcagccttac

SEQ ID NO: 41 - DNA, 19 bp, Artificial Sequence
  Synthetic
gccaatgccaggaagatga

SEQ ID NO: 42 - DNA, 20 bp, Artificial Sequence
  Synthetic
gcgcaacataggatgggttt

SEQ ID NO: 43 - DNA, 30 bp, Artificial Sequence
  Synthetic
tacgaattctccagcggaatgagaacacca

SEQ ID NO: 44 - DNA, 30 bp, Artificial Sequence
  Synthetic
tacggtacccaagatgtacagaagtgcaga

SEQ ID NO: 45 - DNA, 22 bp, Artificial Sequence
  Synthetic
ggaaatacatggcttgctgctt

SEQ ID NO: 46 - DNA, 22 bp, Artificial Sequence
  Synthetic
tctcttcgtcttgatggttgca

SEQ ID NO: 47 - DNA, 20 bp, Artificial Sequence
  Synthetic
caggcttgtatcccaggtca

SEQ ID NO: 48 - DNA, 20 bp, Artificial Sequence
  Synthetic
gcgtaggaggaaagcatgaa

SEQ ID NO: 49 - protein, 17 aa, Artificial Sequence
  Synthetic
AASPGKVLVPDGESDDL

SEQ ID NO: 50 - protein, 10 aa, Artificial Sequence
  Synthetic
AGGPSGEVMI

SEQ ID NO: 51 - protein, 16 aa, Artificial Sequence
  Synthetic
VSAPRDYTMATAEDGV

(19) United States
(12) Patent Application Publication
(10) Pub. No.: US 2019/0338299 A1
(43) Pub. Date: Nov. 7, 2019

(54) HIGH AMYLOSE WHEAT - III

(71) Applicant: Commonwealth Scientific and Industrial Research Organisation, Acton, Australian Capital Territory (AU)

(72) Inventors: Xinguo Li, Duffy, Australian Capital Territory (AU); Zhongyi Li, Kaleen, Australian Capital Territory (AU); Ahmed Regina, Franklin, Australian Capital Territory (AU); Stephen Alan Jobling, Nicholls, Australian Capital Territory (AU)

(21) Appl. No.: 16/312,217

(22) PCT Filed: Jul. 4, 2017
(86) PCT No.: PCT/AU2017/050693

§ 371 Date: Dec. 20, 2018

(30) Foreign Application Priority Data
Jul. 5, 2016 (AU) 2016902643

(51) Int. Cl. C12N 15/82; G01N 33/00; A01H 1/04; A01H 6/46; A23L 7/10

(52) U.S. Cl. CPC C12N 15/8218; G01N 33/0098; A23L 7/197; A01H 6/4678; C12N 15/8245; A01H 1/04

(57) ABSTRACT

The present invention provides wheat grain of the species Triticum aestivum, the grain comprising
i) mutations in each of its SSIIa genes such that the grain is homozygous for a mutation in its SSIIa-A gene, homozygous for a mutation in its SSIIa-B gene and homozygous for a mutation in its SSIIa-D gene, wherein at least two of the mutations in said SSIIa genes are null mutations,
ii) a total starch content comprising an amylose content and an amylopectin content,
iii) a fructan content which is increased relative to wild-type wheat grain on a weight basis, preferably between 3% and 12% of the grain weight,
iv) a β-glucan content,
v) an arabinoxylan content,
vi) a cellulose content.

The grain has a weight of between 25 mg and 60 mg, and the amylose content is between 45% and 70% on a weight basis of the total starch content of the grain as determined by iodine binding assay. The amylopectin content on a weight basis is reduced relative to the wild-type wheat grain, and each of the β-glucan content, arabinoxylan content and cellulose content are increased relative to the wild-type wheat grain on a weight basis, such that the sum of the fructan content, β-glucan content, arabinoxylan content and cellulose content is between 15% and 30% of the grain weight.

### RELATED APPLICATION

This application is a § 371 national stage of PCT International Application No. PCT/AU2017/050693, filed Jul. 4, 2017, and claims priority to Australian patent application no. 2016902643 filed Jul. 5, 2016, the contents of each of which are incorporated herein by reference in their entirety.

### REFERENCE TO A SEQUENCE LISTING

This application incorporates-by-reference nucleotide and/or amino acid sequences which are present in the file named “190710_90774_Sequence_Listing_CAS.txt”, which is 84.1 kilobytes in size, and which was created Jul. 10, 2019 in the IBM-PCT machine format, having an operating system compatibility with MS-Windows, which is contained in the text file filed Jul. 10, 2019 as part of this application.

### FIELD

The specification describes methods of obtaining hexaploid wheat plants having high amylose starch and the use of such plants, and particularly grain or starch therefrom in a range of food and non-food products.

### BACKGROUND

Reference to any prior art in this specification is not, and should not be taken as, an acknowledgment or any form of suggestion that this prior art forms part of the common general knowledge in any country. Throughout this application, various publications are referenced, including referenced in parenthesis. Full citations for publications referenced in parenthesis may be found listed in alphabetical order at the end of the specification immediately preceding the claims. The disclosures of all referenced publications in their entireties are hereby incorporated by reference into this application in order to more fully describe the state of the art to which this invention pertains.

Food produced from wheat grain supplies at least 20% of the food kilojoules for the world population and provides a significant portion of the protein and non-starch polysaccharides as well as energy intake to the human diet. Starch is the major component of wheat grain and is used in a vast range of food and non-food products. Starch characteristics vary and they play a key role in determining the suitability of wheat starch for a particular end use. Despite this huge global consumption and despite an increased awareness of the importance of starch functionality on end product quality, research on genetic variation in wheat and its precise impact on starch characteristics lags behind that for other commercially important plant crops.

Carbohydrate accounts for about 65-75% of the mature wheat grain (Stone and Morell, 2009). The main carbohydrate in wheat grain is starch, which is made up of two polymers of glucose, amylose and amylopectin. Amylose is an essentially linear polymer of α-1,4 linked glucose units with a few branches, while amylopectin is relatively highly branched with α-1,6 glucosidic unit bonds linking linear chains of α-1,4 linked glucose units. The ratio of amylose to amylopectin appears to be a major determinant in (i) the health benefit of wheat grain and wheat starch and (ii) the end quality of products comprising wheat starch.

A second important determinant in wheat grain for health is the amount of non-starch polysaccharide in the grain, which forms part of the dietary fibre. Wild-type wheat grain has about 1% by weight of oligosaccharides such as raffinose, about 1% fructans and about 10% cell wall polysaccharides, mainly cellulose, arabinoxylan and β-glucan (Stone and Morell, 2009). These form the major components of dietary fibre which is not digested and absorbed in the small intestine but passes to the colon where it undergoes bacterial degradation. Dietary fibre is important for regulating blood glucose and insulin levels as well as bowel health.

Cereal grain having starch with increased relative amounts of amylose are of particular interest for their health benefits. Foods comprising increased amylose have been found to be higher in levels of resistant starch (RS), a form of dietary fibre. RS is starch or partially digested starch products that are not digested and absorbed in the small intestine. Resistant starch is increasingly seen to have an important role in promoting intestinal health and in protecting against diseases such as colorectal cancer, type II diabetes, obesity, heart disease and osteoporosis. High amylose starches have been developed in certain cereals such as maize and barley for use in foods as a means of promoting bowel health. The beneficial effects of resistant starch result from the provision of a nutrient to the large bowel wherein the intestinal microflora are given an energy source which is fermented to form short chain fatty acids. These short chain fatty acids provide nutrients for the colonocytes, enhance the uptake of certain nutrients by the large bowel and promote physiological activity of the colon. Generally, if resistant starches or other dietary fibre are not provided to the colon it becomes metabolically relatively inactive. Thus high amylose products have the potential to provide for an increased consumption of fibre. Further potential health benefits of consuming high amylose wheat grains or their products such as starch include an enhanced regulation of sugar, insulin and lipid levels in the blood. Additionally, such foods may promote satiety, improving laxation, promotion of growth of probiotic bacteria and enhancing faecal bile acid excretion.

Most processed starchy foods contain very little RS. Breads made using wild-type wheat flour and conventional formulation and baking processes contain <1% RS. In comparison, breads baked using the same process and storage conditions but containing high amylose wheat flour by virtue of reduced starch branching enzyme activity in the grain had levels of RS as much as 10-fold higher (WO2006/069422). Legumes, which are one of the few rich sources of RS in the human diet, contain levels of RS that are normally <5%. Therefore, consumption of the high amylose wheat bread in amounts normally consumed by adults (e.g. 200 g/d) would readily supply at least 5-12 g of RS. Thus, incorporation of high amylose wheat grain into food products has the potential to make a considerable contribution to dietary RS intakes for humans.

Starch is initially synthesized in plants in chloroplasts of photosynthesizing tissues such as leaves in the form of transitory starch. This is mobilized during subsequent dark periods to supply carbon for export to sink organs and energy metabolism or for storage in organs such as seeds or tubers. Synthesis and long-term storage of starch occurs in the amyloplasts of the storage organs, such as the endosperm of cereals, where the starch is deposited as semicrystalline granules up to 100 μm in diameter. Granules contain both amylose and amylopectin, the former typically as amorphous material in the native starch granule while the latter is semicrystalline through stacking of the linear glucosidic chains. Granules also contain some of the proteins involved in starch biosynthesis.

The synthesis of starch in the endosperm involves at least four types of enzymes (FIG. 1). First, ADP-glucose pyrophosphorylase (ADGP) catalyses the synthesis of ADP-glucose from glucose-1-phosphate and ATP. Secondly, a diverse set of starch synthases (SS; EC 2.4.1.21) catalyse the transfer of glucose residues from ADP-glucose to the non-reducing end by α-1,4 linkages to elongate an α-glucan chain. Thirdly, starch branching enzymes (SBE) form new α-1,6 linkages in α-polyglucans. Lastly, starch debranching enzymes (DBE) then remove some of the branch linkages through a mechanism that has not been fully resolved.

While it is clear that at least these four activities are required for normal starch granule synthesis in higher plants, multiple isoforms of each of the enzymes are found in the endosperm of higher plants. Specific roles for some isozymes have been proposed on the basis of mutational analysis or through the modification of gene expression levels using transgenic approaches (Abel et al., 1996; Jobling et al., 1999; Schwall et al., 2000). However, the contributions of each of the isozymes differs markedly between species and the precise contribution of each isoform to starch biosynthesis are still not known. This is especially true for the hexaploid, bread wheat (Triticum aestivum), which has three sets of homologous chromosomes defining genomes A, B and D. Hexaploidy has been considered a significant obstacle in researching and developing useful variants of wheat. In fact, knowledge is limited regarding how homoeologous genes of wheat interact, how their expression is regulated, and how the different proteins produced by homoeologous genes work separately or in concert.

In maize, rice and wheat, the enzymes starch synthase I (SSI), starch synthase IIa (SSIIa) and starch synthase IIIa (SSIIIa) participate in amylopectin synthesis, perhaps along with other SS. In rice, for example, there are 10 different starch synthases including two granule-bound forms (GBSS). Wheat, barley and rice mutants which are defective for SSIIa have been isolated but the three species show different effects on the phenotypes generated by the loss of SSIIa, especially in the extent of the effects. An ssIIa mutant wheat plant entirely lacking the SGP-1 (SSIIa) protein was produced by crossing lines which were lacking the A, B and D genome specific forms of SGP-1 protein (Yamamori et al., 2000). The ssIIa triple-null grain exhibited deformed starch granules and the starch had altered amylopectin structure. The starch had an amylose content of 30-37% w/w, which was an increase of about 8% over the wild-type level, and a substantial reduction in starch content (Yamamori et al., 2000). Starch from the ssIIa triple-null mutant exhibited a decreased gelatinisation temperature compared to starch from corresponding, wild-type grain. The starch content of the ssIIa triple-null grain was reduced to less than 50% from at least 60% w/w in the wild-type grain. There is no suggestion in Yamamori et al., (2000) that wheat having greater than 45% amylose content in its starch could be produced by combining ssIIa gene mutations, indeed Yamamori et al., teaches to the contrary. This was substantiated by Konik-Rose et al., (2007) who obtained a maximum of 43.98% amylose in the starch of a triple-null ssIIa mutant crossed into wheat variety Sunco.

In barley, a chemically induced null mutation in the SSIIa gene greatly reduced the synthesis of amylopectin and thereby raised the proportion of amylose in grain starch to 65-70% w/w (WO02/37955-A1; Morell et al., 2003). Japonica rice comprises an SSIIa mutation which reduces the SSIIa enzyme in the endosperm relative to Indica rice, but the level of amylose in Japonica grain starch was not substantially elevated compared to Indica grain starch. In rice, a combination of mutations in the SBEIIb and SSIIIa genes had a more substantial effect on the relative amount of amylose (Asai et al., 2014).

The different effects of the ssIIa null mutations in wheat, barley and rice were attributed to different extent of pleiotropic effects of the lack of SSIIa protein on the partitioning of starch synthase I (SSI) and starch branching enzyme IIb (SBEIIb) enzymes inside and outside the starch granules in the developing endosperms of these ssIIa mutants (Luo et al., 2015). In addition, differential effects on post-translational levels of the SSI and SBEIIb proteins may have affected the remaining amylopectin structure. This was one example where observations in one cereal species could not be simply extrapolated to another cereal species in the area of starch synthesis.

In maize and rice, high amylose phenotypes have been generated by mutations in the SBEIIb gene, encoding starch branching enzyme lib, also known as the amylose extender (ae) gene (Boyer and Preiss, 1981, Mizuno et al., 1993; Nishi et al., 2001) rather than an SSIIa gene. In these sbeIIb mutants, the amylose content was significantly elevated as a proportion of the starch content, the branch frequency of the residual amylopectin was reduced and the proportion of short chains (<DP17, especially DP8-12) was reduced. Moreover, the gelatinisation temperature of the starch was increased. To obtain further increases in amylose levels in maize, varieties were produced having reduced starch branching enzyme I (SBEI) activity together with an almost complete inactivation of SBEII activity (Sidebottom et al., 1998).

Wheat having at least 50% amylose as a proportion of the starch content has been generated by reduction in SBEIIa activity alone (Regina et al., 2006) rather than reducing SBEIIb or SSIIa activity. In contrast to maize and rice, wheat having reduced SBEIIb by itself did not yield increased amylose content. International Publication No. WO2005/001098 and International Publication No. WO2006/069422 describe transgenic hexaploid wheat comprising exogenous duplex RNA that reduced expression of one or both of SBEIIa and SBEIIb genes in the endosperm. Grain from transgenic lines expressed reduced levels of SBEIIa and/or SBEIIb proteins. The reduction of SBEIIa protein in endosperm was associated with increased relative amylose levels of more than 50% whereas the lack of SBEIIb protein by itself did not appear to substantially alter the proportion of amylose in grain starch. International Publication Nos. WO2012/058730 and WO2013/063653 report production of non-transgenic sbeIIa and/or sbeIIb triple-null mutants substantially lacking expression of SBEIIa and SBEIIb proteins which exhibited increased amylose levels. The grain of sbeIIa triple-null genotypes were viable provided that at least one of the sbeIIa gene mutations was a point mutation rather than a deletion extending beyond the gene into adjacent regions. Therefore, if it were desired to produce high amylose wheat having at least 50% amylose, the SBEIIa gene is the gene that would be targeted in bread wheat. Levels of non-starch polysaccharides such as fructans were not increased in wheat having reduced SBEII activity and increased (>50%) amylose in its starch (e.g. WO2010/006373).

There is a need in the art for improved high amylose wheat plants and for methods of producing same.

### SUMMARY

The inventors have found, unexpectedly, that hexaploid wheat grain with an amylose content of at least 45%, as a weight percentage of the total starch content of the grain, can be produced by combining mutations in the three SSIIa genes on the A, B and D genomes of the wheat by breeding and selection. The prior art had indicated that 45% amylose was not achievable, indeed the amylose level in ssIIa mutant wheat was generally 30-38% (Yamamori et al., 2000). At least two of the three mutations were null mutations, preferably all three. Since loss of function mutations in SSIIa are recessive, the phenotype was seen when the mutations were in the homozygous state. The inventors also found that the mutant ssIIa wheat grain had significantly increased levels of non-starch polysaccharides, particularly β-glucan, fructan, arabinoxylan and cellulose, each as a percentage of the grain weight. This yielded a substantial increase in total fibre content, as well as associated increases in protein content and other favourable phenotypes.

In a first aspect, the present invention therefore provides a wheat grain of the species Triticum aestivum, the grain comprising
i) mutations in each of its SSIIa genes such that the grain is homozygous for a mutation in its SSIIa-A gene, homozygous for a mutation in its SSIIa-B gene and homozygous for a mutation in its SSIIa-D gene, wherein at least two of the mutations in said SSIIa genes are null mutations; preferably all three are null mutations,
ii) a total starch content comprising an amylose content and an amylopectin content,
iii) a fructan content which is increased relative to wild-type wheat grain on a weight basis, preferably between 3% and 12% of the grain weight,
iv) a β-glucan content,
v) an arabinoxylan content, and
vi) a cellulose content,
   the grain having a grain weight of between 25 mg and 60 mg, wherein the amylose content is between 45% and 70% on a weight basis of the total starch content of the grain as determined by iodine binding assay, wherein the amylopectin content on a weight basis is reduced relative to the wild-type wheat grain, wherein each of the β-glucan content, arabinoxylan content and cellulose content are increased relative to the wild-type wheat grain on a weight basis, such that the sum of the fructan content, β-glucan content, arabinoxylan content and cellulose content is between 15% and 30% of the grain weight.

The present invention further provides wheat plants which are capable of producing, or are obtained from, this grain and products such as flour, bran, wheat starch granules and wheat starch produced from this grain.

The present invention also provides food ingredients comprising the grain of the present invention or material produced from this grain. Also provided are food products including these food ingredients and compositions comprising the grain of the present invention or material produced from this grain. The food ingredient may be kibbled, cracked, par-boiled, rolled, pearled, milled or ground grain or any combination of these. A preferred ingredient is flour, most preferably wholemeal or a blend of wholemeal and white flour. These ingredients have an increased level of total fibre relative to a corresponding ingredient from wild-type wheat by virtue of the incorporation of the material from the wheat grain of the invention.

In another aspect, the present invention provides a process for producing a wheat plant that is capable of producing the grain of the present invention, the process comprising step (i) crossing two parental wheat plants each comprising a null mutation in each of one, two or three SSIIa genes selected from the group consisting of SSIIa A, SSIIa-B and SSIIa-D, or of mutagenising a parental plant, preferably comprising one or two of said null mutations; and step (ii) screening plants or grain obtained from the cross or mutagenesis, or progeny plants or grain obtained therefrom, by analysing DNA, RNA, protein, starch granules or starch from the plants or grain, and step (iii) selecting a fertile wheat plant that has reduced SSIIa activity relative to at least one of the parental wheat plants of step (i).

In another aspect, the present invention provides a process for improving one or more parameters of metabolic health, bowel health or cardiovascular health in a subject in need thereof, or of preventing or reducing the severity or incidence of a metabolic disease such as diabetes, bowel disease or cardiovascular disease, the method comprising providing to the subject the grain or food product of the present invention.

In a still further aspect, the present invention provides a process of producing bins of wheat grain comprising:
a) reaping wheat stalks comprising the wheat grain of the present invention;
b) threshing and/or winnowing the stalks to separate the grain from the chaff; and
c) sifting and/or sorting the grain separated in step b), and loading the sifted and/or sorted grain into bins, thereby producing bins of wheat grain.

In embodiments of each of the above aspects, the wheat grain is further characterised by one or more or all of the features as follows. The amylose content is increased relative to wild-type wheat grain, for example between 48% and 70%, preferably between 50% and 65% of the total starch content of the grain as determined by iodine binding assay. In embodiments, the amylose content is between 50% and 70%, or about 48%, about 50%, about 53%, about 55%, about 60% or about 65%. The starch content of the grain is reduced relative to wild-type wheat grain, for example at least 25%. In embodiments, the starch content of the grain of the invention is between 30% and 70% of the grain weight, between 25% and 65%, between 25% and 60%, between 25% and 55%, between 25% and 50%, between 30% and 70%, between 30% and 65%, between 30% and 60%, between 30% and 55%, or between 30% and 50%. In further embodiments, the starch content is about 35%, about 40%, about 45%, about 50%, about 55%, about 60% or about 65% as a percentage of the grain weight (w/w). In an embodiment, at least 50%, preferably at least 60% or at least 70%, more preferably at least 80% of the starch granules obtained from the grain of the invention show distorted shape and/or surface morphology. The starch of the grain comprises at least 2% resistant starch, preferably at least 3% resistant starch, more preferably between 3% and 15% RS, or between 3% and 10% RS. The starch is characterised by a reduced gelatinisation temperature, which is readily measured by differential scanning calorimetry (DSC), for example the first peak in the DSC scan occurs at a temperature 2-8° C. lower than for wild-type starch. In embodiments, the BG content of the grain is the β-glucan content is increased by 1% or by 2% on an absolute basis relative to wild-type grain and/or is increased by between 2-fold and 7-fold relative to the wild-type wheat grain on a weight basis. In embodiments, the BG level is at least 1% or at least 2%, preferably between 1% and 4% or between 1% and 5% by weight of the grain, preferably about 2%, about 3%, about 4%, more preferably between 2% and 5%. In embodiments, the arabinoxylan content is increased by between 1% and 5% on an absolute basis and/or the cellulose content is increased by between 1% and 5% on an absolute basis. In a preferred embodiment, the grain (prior to any treatment that prevents it germinating) has a germination rate which is between about 70% and about 100% relative to the wild-type wheat grain and the grain, when sown, gives rise to wheat plants which are male and female fertile. Each of these phenotypes are associated with the reduction in SSIIa activity whilst the grain is developing in the wheat plant, the result of the mutations in the SSIIa genes.

The above summary is not and should not be seen in any way as an exhaustive recitation of all embodiments of the present invention.

### BRIEF DESCRIPTION OF THE FIGURES

FIG. 1. Schematic representation of the enzymes involved in starch synthesis in cereal grain, for amylose and amylopectin.

FIG. 2. Schematic gene map of wheat SSIIa-A gene mutation in the A genome of wheat line C57. The upper line shows a map of the exons of the SSIIa-A gene. Below are the nucleotide sequences of a region of the SSIIa-A gene from the wild-type Chinese Spring and mutant C57 showing the deletion of 289 nucleotides in exon 1 including the ATG translation start codon and an insertion of 8 nucleotides, net deletion size of 281 nucleotides. Positions of the primers JKSS2AP1F and JKSS2AP2R are shown.

FIG. 3. Schematic gene map of wheat SSIIa-B gene mutation in the B genome of wheat line K79. The upper line shows a map of the exons of the SSIIa-B gene. Below are the nucleotide sequences of a region of the SSIIa-B gene from the wild-type Chinese Spring (CS) and mutant K79 showing the insertion of 179 nucleotides into exon 8 of SSIIa-B. Positions of the primers JKSS2BP7F and JLTSS2BPR1 are shown.

FIG. 4. Schematic gene map of wheat SSIIa-D gene mutation in the D genome of wheat line Turkey 116. The upper line shows a map of the exons of the SSIIa-D gene. Below are the nucleotide sequences of a region of the SSIIa-D gene from the wild-type Chinese Spring (CS) and mutant T116 showing the deletion of 63 nucleotides spanning the junction of exon 5 and intron 5 (intron 5 splice site) of SSIIa-D. Positions of the primers JTSS2D3F and JTSS2D4R are shown.

FIG. 5. Schematic of the crossing and backcrossing program to produce triple null ssIIa mutants in the Sunco genetic background.

FIG. 6. Schematic of the crossing and backcrossing program to produce triple null ssIIa mutants in the EGA Hume genetic background.

FIG. 7. Schematic of the crossing and backcrossing program to produce triple null ssIIa mutants in the Westonia genetic background.

FIG. 8. Upper panel shows the average grain weight (mg per grain) and the lower panel shows the total lipid content (% weight of the grain) in the triple null ssIIa grain (abd) and wild-type SSIIa grain (WT) for individual wheat lines in the EGA Hume, Sunco and Westonia genetic backgrounds.

FIG. 9. Means of the data in FIG. 8 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 10. Uppermost panel shows the amylopectin content (% of starch content on a weight basis), middle panel shows the amylose content (% of starch content on a weight basis, by iodine binding method) and the lower panel shows the total starch content (% weight of the grain) in the triple null ssIIa grain (abd) and wild-type SSIIa grain (WT) for individual wheat lines in the EGA Hume, Sunco and Westonia genetic backgrounds.

FIG. 11. Means of the data in FIG. 10 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 12. Upper panel shows the total fibre content (% of grain on a weight basis) and the lower panel shows the total BG content (% of grain on a weight basis) in the triple null ssIIa grain (abd) and wild-type SSIIa grain (WT) for individual wheat lines in the EGA Hume, Sunco and Westonia genetic backgrounds.

FIG. 13. Means of the data in FIG. 12 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 14. Uppermost panel shows the fructan content (% of grain on a weight basis), middle panel shows the arabinoxylan content (% of grain on a weight basis) and the lower panel shows the cellulose content (% of grain on a weight basis) in the triple null ssIIa grain (abd) and wild-type SSIIa grain (WT) for individual wheat lines in the EGA Hume, Sunco and Westonia genetic backgrounds.

FIG. 15. Upper panel shows the average grain weight (mg per grain) and the lower panel shows the total lipid content (mg per grain) in the triple null ssIIa grain (abd) and wild-type SSIIa grain (WT) for individual wheat lines in the EGA Hume, Sunco and Westonia genetic backgrounds.

FIG. 16. Means of the data in FIG. 15 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 17. Uppermost panel shows the amylopectin content (mg per grain), middle panel shows the amylose content (mg per grain) and the lower panel shows the total starch content (mg per grain) in the triple null ssIIa grain (abd) and wild-type SSIIa grain (WT) for individual wheat lines in the EGA Hume, Sunco and Westonia genetic backgrounds.

FIG. 18. Means of the data in FIG. 17 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 19. Upper panel shows the total fibre content (mg per grain) and the lower panel shows the total BG content (mg per grain) in the triple null ssIIa grain (abd) and wild-type SSIIa grain (WT) for individual wheat lines in the EGA Hume, Sunco and Westonia genetic backgrounds.

FIG. 20. Means of the data in FIG. 19 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 21. Uppermost panel shows the fructan content (mg per grain), middle panel shows the arabinoxylan content (mg per grain) and the lower panel shows the cellulose content (mg per grain) in the triple null ssIIa grain (abd) and wild-type SSIIa grain (WT) for individual wheat lines in the EGA Hume, Sunco and Westonia genetic backgrounds.

FIG. 22. Means of the data in FIG. 21 for the triple null mutant (abd) and WT genotypes in the EGA Hume, Sunco and Westonia genetic backgrounds, showing the standard deviation. Bars indicated with the same letters (a, b, c) are not statistically significantly different, whereas bars with different letters are significantly different.

FIG. 23. Resistant starch content as a percentage of the starch in three selected triple null ssIIa mutants (Sunco-abd) and wild-type (WT) grain in the Sunco genetic background. The three mutants were not significantly different to each other but were significantly different to WT.

FIG. 24. Average mol % differences of chain length distribution (CLD) profiles of debranched starch from 5 triple ssIIa mutant lines compared with the corresponding wild-type starch. The short chains (DP 6-10) were increased in frequency, while the intermediate chains (DP 11-24) were decreased in frequency for the ssIIa mutant starch compared with that of corresponding wild-type.

FIG. 25. Size exclusion chromatography (SEC) profiles of starch from triple null ssIIa mutant grain and corresponding wild-type wheat starch after isoamylase debranching of the starches. The traces show the distribution of normalized refractometer Index (RI) signals of eluted fractions according to their degree of polymerisation (X-axis). The first eluting peak (I) is amylose, the second (II) is long chain amylopectin and peak III is the (debranched) amylopectin. The black trace was for the ssIIa mutant starch, the grey trace was for wild-type starch.

FIG. 26. Immunological characterisation of starch granule bound proteins from mature wheat grains of mutant ssIIa (B22) and wild-type SSIIa (B70) wheat. The top band in the Western blots was identified as SSIIa, the second band from the top was identified as a mixture of SBEIIa and SBEIIb, and the bands of 70 and 60 kDa were SSI and GBSSI, based on their binding with specific antisera. The identity of each band is labelled and indicated by arrows. The identity of the antibodies used for the different blots are labelled on the left or underneath each panel. M: protein molecular weight marker (kDa).

### LISTING OF THE SEQUENCES

SEQ ID NO:1 Amino acid sequence of SSIIa-A polypeptide, encoded by the wheat A genome; Accession number: AAD53263, 799aa.

SEQ ID NO:2 Amino acid sequence of wheat SSIIa-B polypeptide, encoded by the B genome of wheat; Accession number: CAB96627, 798aa.

SEQ ID NO:3 Amino acid sequence of SSIIa-D polypeptide, encoded by the wheat D genome; Accession number: BAE48800, 799aa; Shimbata et al., (2005).

SEQ ID NO:4 Nucleotide sequence of full length cDNA of wheat SSIIa-A gene; 2821 nucleotides; Accession number: AF155217; Li et al., (1999); translation start codon nucleotides 89-91, stop codon 2486-2488.

SEQ ID NO:5 Nucleotide sequence of full length cDNA of wheat SSIIa-B gene; 2793 nucleotides; Accession number: AJ269504; Gao and Chibbar, (2000); translation start codon nucleotides 135-137, stop codon 2529-2531.

SEQ ID NO:6 Nucleotide sequence of full length cDNA of wheat SSIIa-D gene; 2846 nucleotides; Accession number: AJ269502; Gao and Chibbar, (2000); translation start codon nucleotides 210-212, stop codon 2607-2609. Transit peptide encoded by nucleotides 201-384, mature peptide 385-2606.

SEQ ID NO:7 The nucleotide sequence of the SSIIa-A gene of wheat; Accession number: AB201445; 6898nt (IWGSC: Chromosome 7AS, Traes_7AS_53CAFB43A, 52346437 bp to 52346905 bp, 52351676 to 52351931 bp reverse strand).

SEQ ID NO:8 The nucleotide sequence of the SSIIa-B gene of wheat (Accession number: AB201446) (IWGSC: Chromosome 7DS, Traes_7DS_E6C8AF743, 3877787: 1 to 396 bp, 5137 to 5419 bp forward strand), 6811nt.

SEQ ID NO:9 The nucleotide sequence of the SSIIa-D gene of wheat (Accession number: AB201447) (IWGSC: Chromosome 7DS, Traes_7DS_E6C8AF743, 3877787: 1 to 396 bp, 5137 to 5419 bp forward strand); 6950nt.

SEQ ID NO:10 Amino acid sequence of wheat SSIIb-A encoded in the A genome, 676aa, deduced from the nucleotide sequence of Accession number AK332724.

SEQ ID NO:11 Amino acid sequence of wheat SSIIb-D encoded in the D genome, 674aa, Accession number ABY56824 (which has 100% identity with EU333947).

SEQ ID NO:12 Nucleotide sequence of full length cDNA of wheat SSIIb-A gene on the A genome, Accession number: AK332724. 2727nt (IWGSC: Chromosome 6AL, Traes_6AL_AE01DC0EA, 187,500,495 bp to 187,505,249 bp forward strand).

SEQ ID NO:13 The nucleotide sequence of partial length cDNA of wheat SSIIb-B on the B genome, 1282nt, IWGSC: Chromosome 6DL, gene: Traes_6 BL_61 D83E262, 162,113,784 bp to 162,116,959 bp reverse strand).

SEQ ID NO:14 The nucleotide sequence of full length cDNA of wheat SSIIb-D on the D genome, 2025nt (Accession number: EU333947) (IWGSC: Chromosome 6DL, gene: Traes_6DL_19F1042C7, 147,049,693 bp to 147,051,708 bp reverse strand).

SEQ ID NOs:15-49 Oligonucleotide primers.

SEQ ID NOs:50-51 Amino acid sequences of peptides

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and, may or may not be present depending upon whether or not they affect the activity or action of the listed elements.

As used herein the singular forms “a”, “an” and “the” include plural aspects, and vice versa, unless the context clearly dictates otherwise. Thus, for example, reference to “a mutation” includes a single mutation, as well as two or more mutations; reference to “a plant” includes one plant, as well as two or more plants; and so forth.

As used herein the term “about” in relation to a numerical value or range is intended to cover numbers falling within +10% of the specified numerical value or range.

Each embodiment in this specification is to be applied mutatis mutandis to every other embodiment unless expressly stated otherwise.

Genes and other genetic material (e.g. mRNA, constructs etc) are represented in italics and their proteinaceous expression products are represented in non-italicised form. Thus, for example, SSIIa is an expression product of SSIIa.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided after the claims. A list describing the SEQ ID NOs in the sequence listing is provided after the Figure Legends.

Unless defined otherwise, all technical and scientific terms used herein have the same meaning as commonly understood by those of ordinary skill in the art to which the invention pertains. Although any methods and materials similar or equivalent to those described herein can be used in the practice or testing of the present invention, preferred methods and materials are described.

The present invention is based in part on the surprising observations made in the experiments described herein that hexaploid wheat grain comprising null mutations in each of its three SSIIa genes can be produced which have an amylose content of at least 45% (w/w). This was unexpected on the basis of observations made by others that the level of amylose in triple null ssIIa hexaploid wheat grain was less than 45% (Yamamori et al., 2000; Konik-Rose et al., 2007). In this content, the amylose content is defined as a percentage of the total starch content of the grain on a weight/weight. basis. Furthermore, the wheat grain has other desirable properties including increased total fibre content, based on increases in the fructan, β-glucan, arabinoxylan and cellulose contents, which provides for health benefits when the grain or products produced from the grain are used as food or feed.

Accordingly, in a first aspect the present invention provides a wheat grain of the species Triticum aestivum, the grain comprising
i) mutations in each of its SSIIa genes such that the grain is homozygous for a mutation in its SEIIa-A gene, homozygous for a mutation in its SSIIa-B gene and homozygous for a mutation in its SSIIa-D gene, wherein at least two of the mutations in said SSIIa genes are null mutations,
ii) a total starch content comprising an amylose content and an amylopectin content,
iii) a fructan content which is increased relative to wild-type wheat grain on a weight basis, preferably between 3% and 12% of the grain weight,
iv) a β-glucan content,
v) an arabinoxylan content, and
vi) a cellulose content,
   the grain having a grain weight of between 25 mg and 60 mg, wherein the amylose content is between 45% and 70% on a weight basis of the total starch content of the grain as determined by iodine binding assay, wherein the amylopectin content on a weight basis is reduced relative to the wild-type wheat grain, wherein each of the β-glucan content, arabinoxylan content and cellulose content are increased relative to the wild-type wheat grain on a weight basis, such that the sum of the fructan content, β-glucan content, arabinoxylan content and cellulose content is between 15% and 30% of the grain weight.

The present invention further provides wheat plants which produced or are obtained from this grain and flour and/or wheat starch granules produced from this grain.

The present invention also provides food ingredients comprising the grain of the present invention or material produced from this grain. Also provided are food products including these food ingredients and compositions comprising the grain of the present invention or material produced from this grain. The food ingredient may be kibbled, cracked, par-boiled, rolled, pearled, milled or ground grain or any combination of these.

In another aspect the present invention provides a process for producing a wheat plant that produces the grain of the present invention, the process comprising step (i) crossing two parental wheat plants each comprising a null mutation in each of one, two or three SSIIa genes selected from the group consisting of SSIIa-A, SSIIa-B and SSIIa-D, or of mutagenising a parental plant comprising said null mutations; and step (ii) screening plants or grain obtained from the cross or mutagenesis, or progeny plants or grain obtained therefrom, by analysing DNA, RNA, protein, starch granules or starch from the plants or grain, and step (iii) selecting a fertile wheat plant that has reduced SSIIa activity relative to at least one of the parental wheat plants of step (i).

In another aspect the present invention provides a process for improving one or more parameters of metabolic health, bowel health or cardiovascular health in a subject in need thereof, or of preventing or reducing the severity or incidence of a metabolic disease such as diabetes, bowel disease or cardiovascular disease, the method comprising providing to the subject the grain or food product of the present invention.

As used herein “improving one or more parameters of metabolic health” is a relative term and means an improvement in comparison to the consumption of an equivalent amount of food or drink produced with wild-type wheat.

In a still further aspect the present invention provides a process of producing bins of wheat grain comprising:
a) reaping wheat stalks comprising the wheat grain of the present invention;
b) threshing and/or winnowing the stalks to separate the grain from the chaff; and
c) sifting and/or sorting the grain separated in step b), and loading the sifted and/or sorted grain into bins, thereby producing bins of wheat grain.

In certain embodiments the wheat grain of the present invention is further characterised by one or more or all of the following features:
i) a starch content of between 30% and 70% of the grain weight,
ii) the amylose content is between 45% and 65% of the total starch content of the grain as determined by iodine binding assay,
iii) the starch content has a chain length distribution as determined by fluorescence-activated capillary electrophoresis (FACE) after debranching of the starch samples which is increased in the proportion of chain lengths of DP 7-10 and decreased in the proportion of chain lengths DP 11-24, relative to wild-type wheat starch,
iv) the fructan content comprises fructan of DP 3-12 such that at least 50% of the fructan content is of DP 3-12,
v) the fructan content is increased by between 2-fold and 10-fold relative to the wild-type wheat grain on a weight basis,
vi) the β-glucan content is increased by 1% or by 2% on an absolute basis, and/or is increased by between 2-fold and 7-fold relative to the wild-type wheat grain on a weight basis,
vii) the β-glucan content is between 1% and 4% of the grain weight,
viii) the arabinoxylan content is increased by between PA and 5% on an absolute basis,
ix) the cellulose content is increased by between 1% and 5% on an absolute basis,
x) the grain has a germination rate which is between about 70% and about 100% relative to the wild-type wheat grain, and
xi) the grain, when sown, gives rise to wheat plants which are male and female fertile.

The grain may also comprise a level and/or activity of SSIIa protein which is less than 5% of the level or activity of SSIIa protein in the wild-type wheat grain, or which lacks one or more or all of SSIIa-A protein, SSIIa-B protein and SSIIa-D protein. The grain may also be homozygous for a null mutation in its SSIIa-A gene, homozygous for a null mutation in its SSIIa-B gene and homozygous for a null mutation in its SSIIa-D gene. Each null mutation may be selected, independently, from the group consisting of a deletion mutation, an insertion mutation, a premature translation stop codon, a splice site mutation and a non-conservative amino acid substitution mutation, preferably wherein the grain comprises deletion mutations in each of two or three SSIIa genes, or deletions of the genes entirely.

In certain embodiments the grain further comprises a loss of function mutation in an endogenous gene which encodes a starch synthesis polypeptide, or a chimeric polynucleotide which encodes an RNA which reduces the expression of the endogenous gene which encodes the starch synthesis polypeptide, said starch synthesis polypeptide being selected from the group consisting of SSI, SSIIIa and SSIV, wherein said mutation is selected from the group consisting of a deletion mutation, an insertion mutation, a premature translation stop codon, a splice site mutation and a non-conservative amino acid substitution mutation. It is preferred that at least one, more than one, or all of the mutations are i) introduced mutations, ii) were induced in a parental wheat plant or seed by mutagenesis with a mutagenic agent such as a chemical agent, biological agent or irradiation, or iii) were introduced in order to modify the plant genome.

It is preferred that the grain has an amylose content of about 60% on a weight basis of the total starch content of the grain and/or that the grain is non-transgenic or is free of any exogenous nucleic acid that encodes an RNA which reduces expression of a SSIIa gene and/or a SBEIIa gene.

The SSIIa level and/or activity is determined by assaying the SSIIa level and/or activity in developing endosperm, or by assaying the amount of SSIIa protein in harvested grain by immunological or other means. The endosperm may be either from the plant from which the grain was obtained or a progeny plant.

The starch granules of the grain and/or the starch of the grain of the present invention may also be characterised by one or more of the properties selected from the group consisting of:
i) comprising at least 2% resistant starch;
ii) the starch characterised by a reduced glycaemic index (GI);
iii) the starch granules being distorted in shape;
iv) the starch granules having reduced birefringence when observed under polarized light;
v) the starch characterized by a reduced swelling volume;
vi) modified chain length distribution and/or branching frequency in the starch;
vii) the starch characterized by a reduced peak temperature of gelatinisation;
viii) the starch characterized by a reduced peak viscosity;
ix) reduced starch pasting temperature;
x) reduced peak molecular weight of amylose as determined by size exclusion chromatography;
xi) reduced starch crystallinity; and
xii) reduced proportion of A-type and/or B-type starch, and/or increased proportion of V-type crystalline starch;
   wherein each property is relative to wild-type wheat starch granules or wild-type wheat starch.

In certain embodiments of the present invention the grain is processed so that it is no longer capable of germinating. Examples of such processed grain include heat-treated grain, and kibbled, cracked, par-boiled, rolled, pearled, milled or ground grain. Alternatively, the grain is capable of germinating at a rate between 70% and 100% relative to the wild-type.

The present invention clearly extends to the grain as described above when comprised in a wheat plant. The present invention also extends to wheat plants which produce, or are obtained from, the grain of the present invention. such wheat plants may be characterised by a level and/or activity of SSIIa protein in its endosperm which is less than 5% of the level or activity of SSIIa protein in the wild-type wheat grain, or which lacks one or more or all of SSIIa-A protein, SSIIa-B protein and SSIIa-D protein. Preferably, the wheat plant is male and female fertile.

Flour produced from the grain of the present invention is also encompassed. In an embodiment, the flour is white flour. The flour is preferably wholemeal, or a blend of white flour and wholemeal, for example in a ration from 1:2 to 2:1. The invention also provides wheat bran from the grain of the invention. Each of these products comprise wheat cells having the genetic composition of the wheat grain (i.e the DNA of the wheat grain).

Wheat starch granules or wheat starch produced from the grain is also part of the present invention. The wheat starch granules or wheat starch will typically comprise 45%, preferably about 50%, about 55% or about 60% amylose, or between 45% and 70% amylose, each on a weight basis as a proportion of the total starch content of the starch granules or starch, the starch granules preferably comprising wheat GBSSI polypeptide. The starch granules and/or starch is will also be characterised by one or more of:
a) having no detectable SSIIa polypeptide as determined by an immunological means;
b) comprising at least 2% resistant starch on a weight basis;
c) the starch characterised by a reduced glycaemic index (GI);
d) the starch granules being distorted in shape;
e) the starch granules having reduced birefringence when observed under polarized light;
f) the starch characterized by a reduced swelling volume;
g) modified chain length distribution and/or branching frequency in the starch;
h) the starch characterized by a reduced peak temperature of gelatinisation;
i) the starch characterized by a reduced peak viscosity;
j) reduced starch pasting temperature;
k) reduced peak molecular weight of amylose as determined by size exclusion chromatography;
l) reduced starch crystallinity; and
m) reduced proportion of A-type and/or B-type starch, and/or increased proportion of V-type crystalline starch;
   wherein each property is relative to wild-type wheat starch granules or starch.

The present invention also provides a food ingredient that comprises the grain, the flour, preferably the wholemeal, or wheat bran, or the wheat starch granules or wheat starch of present invention, preferably at a level of at least 10%, preferably about 20% to about 80% on a dry weight basis. The food ingredient may be kibbled, cracked, par-boiled, rolled, pearled, milled or ground grain or any combination of these. The food ingredient may also be incorporated in a food product, preferably at a level of at least 10% on a dry weight basis.

The present invention also provides a composition comprising the wheat grain, the flour, preferably the wholemeal, or wheat bran, or the wheat starch granules or wheat starch of the present invention, at a level of at least 10% by weight, or wheat grain having a level of amylose lower than 45% (w/w) or flour, wholemeal, starch granules or starch obtained therefrom. The composition may comprise a blend of flours.

The present invention also provides a process for producing a food comprising steps of (i) adding a food ingredient of the present invention to another food ingredient, and (ii) mixing the food ingredients, thereby producing the food. The process may also involve processing the grain to produce the food ingredient, prior to step (i), or a step of heating the mixed food ingredients from step (ii) at a temperature of at least 100° C. for at least 10 minutes.

As used herein, the term “by weight” or “on a weight basis” refers to the weight of a substance as a percentage of the weight of the material or item comprising the substance. This is abbreviated herein as “w/w”. For example the amylose content is defined as the weight of amylose as a percentage of the weight of the total starch content.

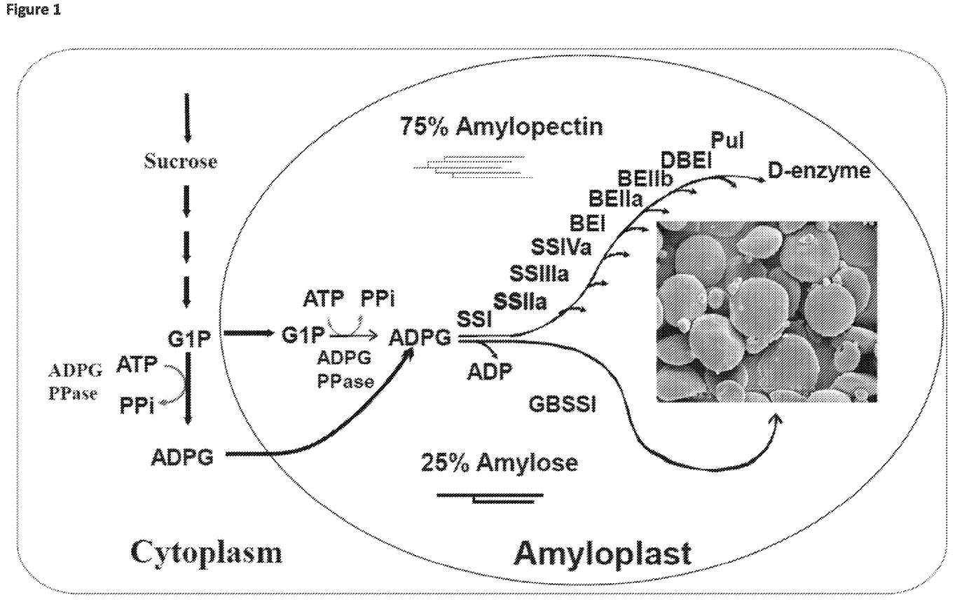

The synthesis of starch in the endosperm of higher plants including wheat is carried out by a suite of enzymes that catalyse four key steps, shown schematically in FIG. 1. Firstly, ADP-glucose pyrophosphorylase (EC 2.7.7.27) activates the monomer precursor of starch through the synthesis of ADP-glucose from G-1-P and ATP. Secondly, the activated glucosyl donor, ADP-glucose, is transferred to the non-reducing end of a pre-existing α-1,4 linkage by starch synthases (EC 2.4.1.24). Thirdly, starch branching enzymes introduce branch points through the cleavage of a region of α-1,4 linked glucan followed by transfer of the cleaved chain to an acceptor chain, forming a new α-1,6 linkage. Starch branching enzymes are the only enzymes that can introduce the α-1,6 linkages into α-polyglucans and therefore play an essential role in the formation of amylopectin. Fourthly, starch debranching enzymes (EC 2.4.4.18) remove some of the branch linkages.

In the cereal endosperm, two isoforms of ADP-glucose pyrophosphorylase (ADGP) are present, one form within the amyloplast, and one form in the cytoplasm. Each form is composed of two subunit types. The shrunken (sh2) and brittle (bt2) mutants in maize represent lesions in large and small subunits respectively.

As used herein, the term “starch synthase” (SS) refers to an enzyme that transfers a glucosyl residue from the activated glucosyl donor, ADP-glucose, to the non-reducing end of a pre-existing glucan chain by a α-1,4 linkage (EC 2.4.1.24). SS enzyme activity may be assayed as described by Guan and Keeling (1998). At least five classes of starch synthase are found in the cereal endosperm including in hexaploid wheat T. aestivum, namely an isoform exclusively localised within or bound to the starch granule, granule-bound starch synthase (GBSS), two forms that are partitioned between the granule and the soluble fraction (SSI and SSII), a form that is entirely located in the soluble fraction (SSIII) and more recently a fifth form, SSIV (FIG. 1). Each of these are included in term “starch synthase”. They are active during endosperm development during growth of the wheat plant, when storage starch is being synthesized and deposited, but may be present in an inactive state in mature (dormant) wheat grain. GBSS has been shown to be essential for amylose synthesis. Each of SSI-IV are involved primarily in amylopectin synthesis on the basis of biochemical and genetic evidence. For example, mutations in SSII and SSIII genes have been shown to alter amylopectin structure (Schondelmaier et al., 1992; Yamamori et al., 2000). Starch synthases are classified according to their amino acid sequence as belonging to one of these five groups based on the extent of homology to known members of these classes.

Within cereals, at least two sub-classes of SSII have been identified, SSIIa and SSIIb, although a third subclass SSIIc has been identified in rice (Ohdan et al., 2005) and genes which appear to encode a corresponding SSIIc enzyme in wheat are identified as described in Example 2. Starch synthase IIa (SSIIa) primarily catalyses the polymerisation of intermediate length glucan chains (DP 12-24) of amylopectin in the endosperm of cereals by transferring a glucosyl moiety from ADP-Glucose to the non-reducing end of a pre-existing α-1,4-linked glucan chains (Fontaine et al., 1993). SSIIa thereby elongates short chains (DP<10) of amylopectin. In a wheat ssIIa mutant, glucan chains of DP 6-11 were increased in frequency and chains of DP 11-25 were decreased in frequency (Yamamori et al, 2000). The different classes of SSII are distinguished by their homology to amino acid sequences of type members of each class i.e. by phylogenetic analysis. Different SSII enzymes and in some cases different wheat SSIIa isoenzymes can be distinguished by the number of amino acids in the polypeptides, as described below.

The level of expression of genes encoding SSII or specifically SSIIa may be assessed by assessing transcript levels such as by Northern blot hybridisation analysis or by RT-PCR analysis. In a preferred method, the amount of SSIIa protein in grain or developing endosperm is measured by separating the proteins in extracts of the grain/endosperm on gels by electrophoresis, then transferring the proteins to a membrane by Western blotting, followed by quantitative detection of the protein on the membrane using specific antibodies (“Western blot analysis”). Exemplary methods for gel electrophoresis and immunoblotting are described in Example 1.

Starch synthase I (SSI) appears to exist as a single isoform in cereals. In rice, SSI accounts for about 70% of the total soluble SS activity in the endosperm (Fujita et al, 2006). SSI preferentially synthesizes short glucan chains of DP6-15, preferring the shortest amylopectin chains as substrates. Despite its important role, the complete absence of SSI enzyme in the rice endosperm does not affect the size and shape of the seed or starch granules, suggesting that other SS enzymes are capable of compensating for absent SSI function.

In contrast, SSIII produces the relatively longer chains of amylopectin, particularly of DP>30, and extends intermediate length glucan chains. ssIII mutants exhibit an increase in intermediate length chains in the amylopectin. There are two forms, SSIIIa being the main form expressed in endosperm, and SSIIIb being a minor form. Little is known about the contribution of SSIV isoforms to glucan chain length in cereal grain, but it appears to function mostly in leaves (Leterrier et al., 2008). Two SSIV genes, SSIVa and SSIVb, are expressed in rice throughout the plant and at relatively constant levels during grain filling, so they appear to have a function throughout the plant. ssIV mutants in Arabidopsis had decreased levels of leaf starch.

Each of the starch synthases are expressed as polypeptides with N-terminal signal peptides that are cleaved off during translocation into the amyloplasts.

As used herein, “starch branching enzyme” (SBE) means an enzyme that introduces α-1,6 glycosidic bonds between chains of glucose residues (EC 2.4.1.18), thereby introducing the α-1,6 branch points in amylopectin. Two main classes of SBEs are known in plants, SBEI and SBEII. SBEII can be further categorized into two types in cereals, SBEIIa and SBEIIb (Redman and Boyer, 1982; Boyer and Preiss, 1978; Mizuno et al., 1992, Sun et al., 1997). Additional forms of SBEs are also reported in some cereals, a putative 149 kDa SBEI from wheat and a 50/51 kDa SBE from barley. Sequence alignment revealed a high degree of sequence similarity at both the nucleotide and amino acid levels and allows the grouping into the SBEI, SBEIIa and SBEIIb classes. The amino acid sequences of SBEIIa and SBEIIb generally exhibit around 80% identity to each other, mostly concentrated in the central regions of the polypeptides.

SBEI, SBEIIa and SBEIIb may also be distinguished by their expression patterns, but this differs in different species. In wheat endosperm, SBEI (Morell et al, 1997) is found exclusively in the soluble fraction, while SBEIIa and SBEIIb are found in both soluble and starch-granule associated fractions (Rahman et al., 1995). In maize, SBEIIb is the predominant form in the endosperm whereas SBEIIa is expressed relatively more strongly in the leaf and appears to be expressed throughout the plant (Gao et al., 1997). In rice, SBEIIa and SBEIIb are found in the endosperm in approximately equal amounts. However, there are also differences in timing of gene expression. SBEIIa is expressed at an earlier stage of seed development, being detected at 3 days after flowering, and was expressed in leaves, while SBEIIb was not detectable at 3 days after flowering and was most abundant in developing seeds at 7-10 days after flowering and was not expressed in leaves. In wheat endosperm, SBEIIa is expressed about 3-4-fold more highly than SBEIIb. Different cereal species show significant differences in SBEIIa and SBEIIb expression, and conclusions drawn in one species cannot readily be applied to another species. Specific antibodies may also be used to distinguish the enzymes.

Genomic and cDNA sequences for each of the SBE genes have been characterized, including for wheat. Sequence alignment reveals a high degree of sequence similarity at both the nucleotide and amino acid levels, but also the sequence differences and allows the grouping into the SBEI, SBEIIa and SBEIIb classes. In wheat, apparent gene duplication events have increased the number of SBEI genes in each genome (Rahman et al., 1999). The elimination of greater than 97% of the SBEI activity in wheat endosperm by combining mutations in the highest expressing forms of the SBEI genes from the A, B and D genomes had no measurable impact on starch structure or functionality (Regina et al., 2004). In contrast, reduction of SBEIIa expression by a gene silencing construct in hexaploid wheat resulted in high amylose levels (>70%), while a corresponding construct that reduced SBEIIb expression but not SBEIIa had minimal effect (Regina et al., 2006). In barley, a gene silencing construct which reduced both SBEIIa and SBEIIb expression in endosperm was used to generate high amylose barley grain (Regina et al., 2010). In maize, SBEIIb mutants known as amylase extender (ae) produced high amylose phenotypes.

Enzyme activity assays of branching enzymes to detect the activity of all three isoforms, SBEI, SBEIIa and SBEIIb are based on the method of Nishi et al., 2001 with minor modification as follows. After electrophoresis, the gel is washed twice in 50 mM HEPES, pH 7.0 containing 10% glycerol and incubated at room temperature in a reaction mixture consisting of 50 mM HEPES, pH 7.4, 50 mM glucose-1-phosphate, 2.5 mM AMP, 10% glycerol, 50 U phosphorylase a, 1 mM DTT and 0.08% maltotriose for 16 h. The bands are visualised with a solution of 0.2% (w/v) 12 and 2% KI. The SBEI, SBEIIa and SBEIIb isoform-specific activities are separated under these conditions of electrophoresis. This is confirmed by immunoblotting using anti-SBEI, anti-SBEIIa and anti-SBEIIb antibodies. Densitometric analysis of immunoblots which measures the intensity of each band is conducted to determine the level of enzyme activity of each isoform.

Starch branching enzyme (SBE) activity may be measured by enzyme assay, for example by the phosphorylase stimulation assay (Boyer and Preiss, 1978). This assay measures the stimulation by SBE of the incorporation of glucose 1-phosphate into methanol-insoluble polymer (α-D-glucan) by phosphorylase A. Isoforms of SBE show different substrate specificities, for example SBEI exhibits higher activity in branching amylose, while SBEIIa and SBEIIb show higher rates of branching with an amylopectin substrate. SBEI preferentially produces longer chains with DP>16 by branching less branched polyglucans, whereas SBEII isoenzymes generates shorter chains with DP<12. The isoforms may also be distinguished on the basis of the length of the glucan chain that is transferred.

Two classes of debranching enzyme (DBE) are known in cereals, namely isoamylase (ISA) and pullulanase (PUL). ISA mainly debranches phytoglycogen and amylopectin, whereas PUL acts upon pullulan and amylopectin but not phytoglycogen (Nakamura et al., 1996). Mutants for ISA have been termed sugary and produce more short chains of amylopectin, and ISA therefore appears to act in the editing of excessively branched chains or improper branches in amylopectin. In contrast, PUL is believed to function in starch degradation in germinating grain as well as starch synthesis.

Developing hexaploid wheat endosperm expresses SSIIa from the SSIIa genes on each of the A, B and D genomes. As used herein, “SSIIa expressed from the A genome” or “SSIIa-A” means a polypeptide whose amino acid sequence is set forth in SEQ ID NO:1 or which is at least 99% identical to the amino acid sequence set forth in SEQ ID NO: 1 or comprising such a sequence. The amino acid sequence provided as SEQ ID NO:1 (Li et al., 1999; Genbank Accession No. AAD53263) is used herein as the reference sequence for a wild-type SSIIa-A polypeptide. The polypeptide of SEQ ID NO:1 is 799 amino acid residues long, as would amino acid substitution mutants of SEQ ID NO:1. Enzymatically active variants of this enzyme exist in wheat, for example in cultivar Fielder, see Accession No. CAB96626.1 (Gao and Chibbar, 2000) whose amino acid sequence is 99.5% (795/799) identical to SEQ ID NO.1, and in diploid relatives of Triticum aestivum such as from Triticum urartu, provided as Accession Nos. CUS28065.1 and CDI68213.1. Such variants are included in “SSIIa-A”. SSIIa-A does not include the homoeologous polypeptides, SSIIa-B and SSIIa-D, because those polypeptides are about 96% identical to SEQ ID NO:1.

As used herein, “SSIIa expressed from the B genome” or “SSIIa-B” means a polypeptide whose amino acid sequence is set forth in SEQ ID NO:2 or which is at least 99% identical to the amino acid sequence set forth in SEQ ID NO:2 or comprising such a sequence. The amino acid sequence provided as SEQ ID NO:2 (Genbank Accession No. CAB99627.1) corresponds to the starch synthase IIa expressed from the B genome of wheat variety Fielder, which is used herein as the reference sequence for a wild-type SSIIa-B polypeptide. The polypeptide of SEQ ID NO:2 is 798 amino acids long, as would amino acid substitution mutants of SEQ ID NO:2. Enzymatically active variants of this enzyme exist in wheat, such variants are included in “SSIIa-B”. SSIIa-B does not include the homoeologous polypeptides, SSIIa-A and SSIIa-D, because those polypeptides are about 96% identical to SEQ ID NO:2 (Li et al., 1999).

As used herein, “SSIIa expressed from the D genome” or “SSIIa-D” means a polypeptide whose amino acid sequence is set forth in SEQ ID NO:3 or which is at least 99% identical to the amino acid sequence set forth in SEQ ID NO:3 or comprising such a sequence. The amino acid sequence of SEQ ID NO:3 (Genbank Accession No. BAE48800; Shimbata et al., 2005) corresponds to the SSIIa expressed from the D genome in wheat cultivar Chinese Spring, which is used herein as the reference sequence for wild-type SSIIa-D. The protein of SEQ ID NO:3 is 799 amino acids long. Enzymatically active variants of this enzyme exist in wheat, for example in cultivar Fielder, see Accession No. CAB86618 (Gao and Chibbar, 2000) whose amino acid sequence is 99.9% (798/799) identical to SEQ ID NO.3, and in diploid relatives of Triticum aestivum such as Aegilops tauschii, a likely progenitor of the D genome of hexaploid wheat, provided as Accession No CAB86618. Such variants are included in “SSIIa-D”. SSIIa-D does not include the homoeologous polypeptides, SSIIa-A and SSIIa-B, because those polypeptides are about 96% identical to SEQ ID NO:3. The amino acid sequence provided as SEQ ID NO: 3 is 95.9% identical to each of SEQ ID NO:1 and SEQ ID NO:2. Alignment of the three amino acid sequences shows amino acid differences which may be used to distinguish the proteins or to classify variants as SSIIa-A, SSIIa-B or SSIIa-D.

When comparing amino acid sequences to determine the percentage identity in this context, for example by Blastp, the full length sequences should be compared, and gaps in a sequence counted as amino acid differences.

As used herein, an “SSIIa polypeptide” means an SSIIa-A polypeptide, SSIIa-B polypeptide or SSIIa-D polypeptide.

As used herein, each of the SSIIa-A, SSIIa-B and SSIIa-D polypeptides include polypeptide variants which have reduced or no starch synthase enzyme activity, as well as polypeptides having wild-type or essentially wild-type enzyme activity. Comparison of the amino acid sequence of a mutant form of an SSIIa polypeptide with SEQ ID NOs:1, 2 and 3 is used to determine which of the SSIIa-A, -B or -D polypeptides it is derived from and so to classify the mutant form. For example, a mutant SSIIa polypeptide is considered to be a mutant SSIIa-A polypeptide if its amino acid sequence is more closely related, i.e. having a higher degree of sequence identity, to SEQ ID NO:1 than to SEQ ID NOs:2 and 3. Analogously, a mutant SSIIa polypeptide is a mutant SSIIa-B polypeptide if it is more closely related to SEQ ID NO:2 than SEQ ID NOs:1 or 3, and a mutant SSIIa polypeptide is a mutant SSIIa-D polypeptide if it is more closely related to SEQ ID NO:3 than SEQ ID NOs:1 and 2. Those skilled in the art are thereby able to classify a mutant SSIIa polypeptide.

A mutant SSIIa polypeptide may have reduced starch synthase enzyme activity (a partial mutant) or lacks starch synthase activity (null mutant polypeptide). A mutant SSIIa gene may be expressed to produce an SSIIa polypeptide in wheat endosperm, for example a truncated polypeptide, or it may not be expressed, and produce no polypeptide at all. It may also be expressed to produce a transcript but no translation product.

It is also understood that SSIIa proteins may be present in grain, particularly mature grain as commonly harvested commercially, but in an inactive or dormant state because of the physiological conditions in the grain. Such polypeptides are included in “SSIIa polypeptides” as used herein. The SSIIa polypeptides may be enzymatically active during only part of grain development, in particular in developing endosperm when storage starch is typically deposited, but in an inactive state otherwise. Such SSIIa polypeptides may be detected and quantitated readily using immunological methods such as Western blot analysis.

Thus, “wild-type” as used herein when referring to an SSIIa-A polypeptide means a polypeptide whose amino acid sequence is set forth in SEQ ID NO: 1 or enzymatically active variants at least 99% identical in amino acid sequence which are found in nature and which have essentially the same activity as SEQ ID NO:1; “wild-type” as used herein when referring to SSIIa-B means a polypeptide whose amino acid sequence is set forth in SEQ ID NO: 2 or enzymatically active variants at least 99% identical in amino acid sequence which are found in nature and which have essentially the same activity as the polypeptide whose sequence is provided as SEQ ID NO:2; “wild-type” as used herein when referring to SSIIa-D means a polypeptide whose amino acid sequence is set forth in SEQ ID NO: 3 or enzymatically active variants at least 99% identical in amino acid sequence which are found in nature and which have essentially the same activity as the polypeptide whose sequence is provided as SEQ ID NO:3. In each case, the wild-type polypeptide has starch synthase II activity and has not been modified by the present invention.

Wild-type wheat produces two other classes of SSII polypeptides, namely SSIIb and SSIIc polypeptides. As used herein, “SSIIb expressed from the A genome” or “SSIIb-A” means a polypeptide whose amino acid sequence is set forth in SEQ ID NO:10 or which is at least 99% identical to the amino acid sequence set forth in SEQ ID NO:10 or comprising such a sequence. The amino acid sequence provided as SEQ ID NO:10 (deduced from the nucleotide sequence of Genbank Accession No. AK332724) is used herein as the reference sequence for a wild-type SSIIb-A polypeptide. The polypeptide of SEQ ID NO:10 is 676 amino acid residues long. Enzymatically active variants of this enzyme are included in “SSIIb-A”. SSIIb-A does not include the homoeologous polypeptide SSIIb-D which is about 90% identical to SEQ ID NO:8.

As used herein, “SSIIb expressed from the D genome” or “SSIIb-D” means a polypeptide whose amino acid sequence is set forth in SEQ ID NO:11 or which is at least 99% identical to the amino acid sequence set forth in SEQ ID NO:11 or comprising such a sequence. The amino acid sequence provided as SEQ ID NO:11 (Genbank Accession No. ABY56824) corresponds to the starch synthase lib expressed from the D genome of bread wheat, which is used herein as the reference sequence for a wild-type SSIIb-D polypeptide. The polypeptide of SEQ ID NO:11 is 674 amino acids long. Enzymatically active variants of this enzyme are included in “SSIIb-D”. SSIIb-D does not include the homoeologous polypeptide SSIIb-A which is about 90% identical to SEQ ID NO:11.

The amino acid sequences of the SSIIa homoeologs and the SSIIb homoeologs are 71-79% identical, and therefore the polypeptides can be readily distinguished even though they have similar enzyme activities.

As described in Example 2 herein, wheat SSIIc sequences were also identified and could be readily distinguished from the SSIIa sequences.

As used herein, the terms “SSIIa gene” and “wheat SSIIa gene” and the like refer to the genes that encode a SSIIa polypeptide, including wild-type SSIIa polypeptides such as homologous polypeptides present in other wheat varieties as well as mutant forms of the genes which either encode SSIIa polypeptides with reduced activity or undetectable activity, or genes which were derived therefrom by mutation. SSIIa genes include, but are not limited to, the wheat SSIIa genes which have been cloned, including the genomic and cDNA sequences listed in Table 1 which are annotated as SSIIa genes. The term SSIIa gene includes, collectively, each of the more specific terms “SSIIa-A gene”, “SSIIa-B gene” and “SSIIa-D gene”, encoding an SSIIa-A, SSIIa-B and SSIIa-D polypeptide, respectively, or mutant forms derived from such genes. The SSIIa genes as used herein encompasses mutant forms which do not encode any polypeptide at all, or polypeptides which have no starch synthase activity, in which cases the mutant forms represent null alleles of the genes. Alleles of the genes include mutant alleles where at least part of the gene is deleted, including where the entire gene is deleted, which alleles also represent null alleles of the genes.

An “endogenous SSIIa gene” refers to an SSIIa gene which is in its native location in the wheat genome, including wild-type and mutant forms. As is understood in the art, hexaploid wheats such as bread wheat comprise three genomes which are commonly designated the A, B and D genomes, while tetraploid wheats such as durum wheat comprise two genomes commonly designated the A and B genomes. Each genome comprises 7 pairs of chromosomes which may be observed by cytological methods during meiosis and thus identified, as is well known in the art. Endogenous SSIIa-A genes, SSIIa-B genes and SSIIa-D genes are located on the short arm of chromosomes 7A, 7B and 7D, respectively, in hexaploid wheat. In contrast, the terms “isolated SSIIa gene” and “exogenous SSIIa gene” refer to an SSIIa gene which is not in its native location, for example having been removed from a wheat plant, cloned, synthesized, comprised in a vector or in the form of a transgene in a cell, such as transgene in a transgenic wheat plant. The SSIIa gene in this context may be any of the specific forms as described as follows.

TABLE 1
Starch synthase enzyme genes characterized from cereals

| | SS | Type of | | |
|---|---|---|---|---|
| Species | isoform | clone | Accession No. | Reference |
| Wheat | SSI | cDNA and | AF091803 (cDNA) | Li et al., 1999 |
| | | genomic | AF091802 (genomic) | Li et al., 1999 |
| | SSIIa-A | cDNA and | AF155217 (cDNA) | Li et al., 1999 |
| | | genomic | AB201445 (genomic) | Shimbata et al., 2005 |
| | SSIIa-B | cDNA and | AJ269504 (cDNA) | Gao and Chibbar, 2000 |
| | | genomic | AB201446 (genomic) | Shimbata et al., 2005 |
| | SSIIa-D | cDNA and | AJ269502 (cDNA) | Gao and Chibbar, 2000 |
| | | genomic | AB201447 (genomic) | Shimbata et al., 2005 |
| | SSIIb-A | cDNA and | AK332724 (cDNA) | Kawaura et al., 2009 |
| | | genomic | Traes_6AL_AE01DC0EA, | The IWGSC databases |
| | | | 6A: 187503405 bp to | |
| | | | 187505233 bp (genomic) | |
| | SSIIb-B | cDNA and | Traes_6BL_61D83E262, | The IWGSC databases |
| | | genomic | 6B: 162116364-162116691 | |
| | | | bp (genomic) | |
| | SSIIb-D | cDNA and | EU333947 (cDNA) | NCBI |
| | | genomic | Traes_6DL_19F1042C7, 6D: | The IWGSC databases |
| | | | 147050072-147051031 bp | |
| | | | (genomic) | |
| | SSIIc-A | cDNA and | Traes_1AL_729BF3204, 1A: | The IWGSC databases |
| | | genomic | 68687585-68688377, | |
| | SSIIc-B | cDNA and | Traes_1BL_447468BDE, 1B: | The IWGSC databases |
| | | genomic | 31475067-314776087 bp | |
| | SSIIc-D | cDNA and | EU307274 (cDNA) | NCBI |
| | | genomic | Traes_1DL_F667ED844, | The IWGSC databases |
| | | | IWGSC_CSS_1DL_scaff_22 | |
| | | | 05619: 1950-3041 bp | |
| | SSIIIa | cDNA | AF258608 (cDNA) | Li et al., 2000 |
| | | | AF258609 (genomic) | Li et al., 2000 |
| | SSIIIb | cDNA and | EU333946 (cDNA) | NCBI |
| | | genomic | | |
| | SSIVa | cDNA | AY044844 (cDNA) | NCBI |
| | | | DQ400416 (genomic) | Leterrier et al., 2008 |
| Rice | SSIIa | cDNA | AF419099 (cDNA) | NCBI |
| | SSIIb | cDNA | AF395537 (cDNA) | NCBI |
| | SSIIc | cDNA | AF383878 (cDNA) | NCBI |
| Barley | SSIIa | cDNA | AY133249 (cDNA) | Li et al., 2003 |
| | SSIIb | cDNA | AK372518 (cDNA) | Matsumoto et al., 2011 |
| | SSIIc | cDNA | AK372414 (cDNA) | Matsumoto et al., 2011 |
| Maize | SSIIa | cDNA | AF019296 (cDNA) | Harn et al., 1998 |
| | SSIIb | cDNA | NM_001112544 (cDNA) | Schnable et al., 2009 |
| | SSIIc | cDNA | EU284113 (cDNA) | Yan et al., 2008 |
| Arabidopsis | SSII | cDNA | NM_110984 (cDNA) | Salanoubat et al., 2000 |

As used herein, “a SSIIa gene on the A genome of wheat” or “SSIIa-A gene” means any polynucleotide which encodes an SSIIa-A polypeptide as defined herein or which is derived from a polynucleotide which encodes SBEIIa-A in a wheat plant, including naturally occurring polynucleotides, sequence variants or synthetic polynucleotides, including “wild-type SSIIa-A gene(s)” which encode an SSIIa-A polypeptide with essentially wild-type SSIIa activity, and “mutant SSIIa-A gene(s)” which do not encode an SSIIa-A polypeptide with essentially wild-type activity but which are recognizably derived from a wild-type SSIIa-A gene. Comparison of the nucleotide sequence of a mutant form of an SSIIa gene with a suite of wild-type SSIIa genes is used to determine which of the SSIIa genes it is derived from and so to classify it. For example, a mutant SSIIa gene is considered to be a mutant SSIIa-A gene if its nucleotide sequence is more closely related, i.e. having a higher degree of sequence identity, to a wild-type SSIIa-A gene than to any other SSIIa gene. A mutant SSIIa-A gene encodes an SSIIa polypeptide with reduced starch synthase enzyme activity (partial mutant), or a polypeptide which lacks starch synthase activity or no protein at all (null mutant gene). An exemplary nucleotide sequence of a cDNA corresponding to an SSIIa-A gene is given in SEQ ID NO:4 (Genbank Accession No. AF155217; Li et al., 1999). Other exemplary nucleotide sequences are provided in Accession Nos. AK330838 (cDNA from SSIIa-A gene from cultivar Chinese Spring, Kawaura et al., 2009), and Accession No. AJ269503 which provides a cDNA from an SSIIa-A gene from cultivar Fielder (Gao and Chibbar, 2000).

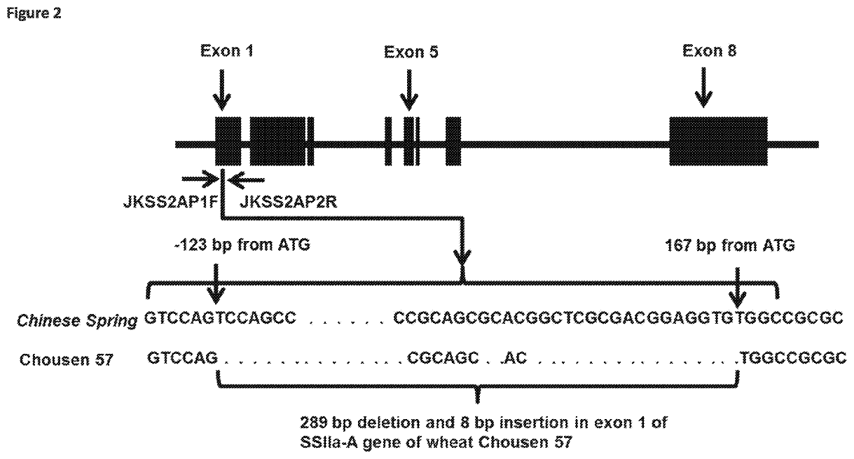

As used herein, the terms “SSIIa gene on the B genome” or “SSIIa-B gene”, and “SSIIa gene on the D genome” or “SSIIa-D gene” have corresponding meanings to that for SSIIa-A in the previous paragraph. An exemplary nucleotide sequence of a cDNA corresponding to an SSIIa-B gene is given in SEQ ID NO:5 (Genbank Accession No. AJ269504; Gao and Chibbar 2000) and of an SSIIa-D gene is given in SEQ ID NO:6 (Genbank Accession No. AJ269502; from cultivar Fielder, Gao and Chibbar, 2000). Sequences of parts of SSIIa genes are also given herein as referred to in FIGS. 2-4.

As used herein, “a SSIIb gene on the A genome of wheat” or “SSIIb-A gene” means any polynucleotide which encodes an SSIIb-A polypeptide as defined herein or which is derived from a polynucleotide which encodes SSIIb-A in a wheat plant, including naturally occurring polynucleotides, sequence variants or synthetic polynucleotides, including “wild-type SSIIb-A gene(s)” which encode an SSIIb-A polypeptide with essentially wild-type SSIIb activity, and “mutant SSIIb-A gene(s)” which do not encode an SSIIb-A polypeptide with essentially wild-type activity but which are recognizably derived from a wild-type SSIIb-A gene. Comparison of the nucleotide sequence of a mutant form of an SSIIb gene with a suite of wild-type SSIIb genes is used to determine which of the SSIIb genes it is derived from and so to classify it. For example, a mutant SSIIb gene is considered to be a mutant SSIIb-A gene if its nucleotide sequence is more closely related, i.e. having a higher degree of sequence identity, to a wild-type SSIIb-A gene than to any other SSIIb gene. A mutant SSIIb-A gene encodes an SSIIb polypeptide with reduced starch synthase enzyme activity (partial mutant), or a polypeptide which lacks starch synthase activity or no protein at all (null mutant gene). An exemplary nucleotide sequence of a cDNA corresponding to an SSIIb-A gene is given in SEQ ID NO:12 (Genbank Accession No. AK332724).

As used herein, the terms “SSIIa gene on the B genome” or “SSIIa-B gene”, and “SSIIa gene on the D genome” or “SSIIa-D gene” have corresponding meanings to that for SSIIa-A in the previous paragraph. An exemplary nucleotide sequence of a cDNA corresponding to an SSIIa-B gene is given in SEQ ID NO:5 (Genbank Accession No. AJ269504; Gao and Chibbar 2000) and of an SSIIa-D gene is given in SEQ ID NO:6 (Genbank Accession No. A7269502; from cultivar Fielder, Gao and Chibbar, 2000).

The SSIIa genes as defined above include any regulatory sequences that are 5′ or 3′ of the transcribed region, including the promoter region, that regulate the expression of the associated transcribed region, and introns within the transcribed regions. An exemplary nucleotide sequence of an SSIIa gene is provided as SEQ ID NO:7, which provides the nucleotide sequence of an SSIIa-A gene from the A genome of wheat; Accession No. AB201445. In analogous fashion, the nucleotide sequence of a wild-type SSIIa-B gene of wheat is provided as Accession number AB201446 (IWGSC: Chromosome 7DS, Traes_7DS_E6C8AF743, 3877787: 1 to 396 bp, 5137 to 5419 bp forward strand) and of an SSIIa-D gene of wheat is provided as Accession number AB201447 (IWGSC: Chromosome 7DS, Traes_7DS_E6C8AF743, 3877787: 1 to 396 bp, 5137 to 5419 bp forward strand). Each of these wild-type genes were from wheat cultivar Chinese Spring (Shimbata et al., 2005).

It would be understood that there is natural variation in the sequences of SSIIa genes from different wheat varieties. The homoeologous genes are readily recognizable by the skilled artisan on the basis of sequence identity. The degree of sequence identity between the nucleotide sequences of the homoeologous wild-type SSIIa genes and the amino acid sequences of the wild-type polypeptides is 95-96%.

An allele is a variant of a gene at a single genetic locus. A diploid organism has two sets of chromosomes. Hexaploid wheat was six sets of chromosomes, 7 chromosomes in each set and is thought to have arisen by hybridisation of three progenitor diploid plants contributing the A-, B- and D-genomes. Each chromosome of a pair of chromosomes has one copy (i.e. one allele) of each gene. If both alleles of a gene are the same, the organism is homozygous with respect to that allele or gene. If the two alleles of a gene are different, the organism is heterozygous with respect to that gene. The interaction between alleles at a locus is generally described as dominant or recessive. The two alleles of a gene in the wheat plant or grain may have the same mutation as each other, so are said to be homozygous for that mutation, or the two alleles may comprise different mutations to each other and are said to be heterozygous for those mutations. Different alleles of a gene, or for multiple genes, may be combined using methods known in the art. For example, two parental wheat plants which have different alleles for a gene may be crossed to produce progeny (F1) which contain both alleles, in the heterozygous state, and the progeny plants then self-fertilised to produce a further generation of plants (F2) which comprise one or the other of the alleles in the homozygous state or both alleles in the heterozygous state, according to Mendelian genetics.

Alleles that do not encode or are not capable of leading to the production of any active enzyme are null alleles. Such null alleles may or may not encode a polypeptide, for example encoding a truncated polypeptide or a polypeptide having an inactivating change in amino acid sequence relative to a wild-type SSIIa polypeptide.

Reference to a null mutation(s) includes a null mutation independently selected from the group consisting of a deletion mutation, an insertion mutation, a splice-site mutation, a premature translation termination mutation, and a frameshift mutation, or any combination thereof. In an embodiment, one or more of the null mutations are non-conservative amino acid substitution mutations or a null mutation has a combination of two or more non-conservative amino acid substitutions. In this context, non-conservative amino acid substitutions are as defined herein.

A loss of function mutation, which includes a partial loss of function mutation in an allele of a gene as well as complete loss of function mutation (null mutation), means a mutation in the allele leading to a reduced level or activity of the enzyme, such as an SSIIa enzyme in the grain. The mutation in the allele may mean, for example, that less protein having wild-type or reduced activity is translated or that wild-type or reduced levels of transcription are followed by translation of an enzyme with reduced enzyme activity, or preferably the mutant allele is both transcribed at a reduced rate compared to wild-type and any translation product has less activity than the corresponding wild-type polypeptide. The mutation may result in, for example, that no or less RNA is transcribed from the gene comprising the mutation or that the polypeptide that is produced has no or reduced activity relative to wild-type, preferably both. If there is no transcript detected from an allele, for example by RT-PCR assay, that result indicates that the allele is a null allele.

A “point mutation” refers to a single nucleotide base change which includes a deletion, substitution or insertion of a single nucleotide. The point mutation may further be a splice-site mutation, a premature translation termination mutation, a frame shift mutation or other loss of function mutation wherein the mutation results in no protein being produced or the protein is produced in lower amounts or the protein produced has lower SSII activity. A frame shift mutation in a protein coding region of a gene is considered a null mutation because of its effect on the structure of the encoded polypeptide. Likewise, a premature translation termination mutation is considered a null mutation unless it occurs very close to the C-terminus of the protein coding region of the gene, in which case an enzyme assay can be used to determine whether the polypeptide has enzyme activity. In some embodiments, the point mutation results in a conservative or preferably a non-conservative amino acid substitution.

A “reduced” or “lower” amount or level of polypeptide or enzyme activity means a reduced or lower amount or level relative to the amount or level produced by the corresponding wild-type allele or gene. Typically, the reduction is by at least 40%, preferably at least 50% or at least 60%, more preferably at least 80% or 90% relative to the wild-type. In a most preferred embodiment, the protein is not detected, such as for example in a Western blot assay as described herein, preferably for each of SSIIa-A, SSIIa-B and SSIIa-D.

A “reduced” activity means reduced relative to the corresponding wild-type enzyme, such as an SSIIa, SSSIIa or other enzyme.

Protein “activity” refers to SS activity which may be measured directly or indirectly by various means known in the art and as described herein.

In some embodiments, the amount by weight of an SSIIa or other polypeptide is reduced even though the number of polypeptide molecules in the grain is the same as in the wild-type, but each molecule having less activity than the wild-type. For example, the polypeptides produced are shorter than wild-type SSIIa protein or other protein, such as occurs if the mutant SSIIa protein or other protein is truncated due to a premature translation termination signal.

As used herein, “two identical alleles of an SSIIa-A gene”, means that the two alleles of the SSIIa-A gene are identical to each other i.e the plant or grain is homozygous for those alleles or that gene; “two identical alleles of an SSIIa-B gene”, means that the two alleles of the SSIIa-B gene are identical to each other; “two identical alleles of an SSIIa-D gene”, means that the two alleles of the SSIIa-D gene are identical to each other.

The wheat plants of the invention can be produced and identified after mutagenesis. In some embodiments, the wheat plant is non-transgenic, which is desirable in some markets, or which is free of any exogenous nucleic acid molecule which reduces expression of an SSIIa gene. In another embodiment, the wheat plant is transgenic, for example it comprises an exogenous nucleic acid molecule other than one which reduces expression of an SSIIa gene and/or an SBEIIa gene, such as for example, an exogenous nucleic acid molecule which encodes a polypeptide that confers herbicide tolerance to the plant.

Mutant wheat plants having a mutation in a single SSIIa gene which can be combined by crossing of plants and selection of progeny having other SSIIa gene mutations to generate the wheat plants of the invention can be either synthetic, for example, by performing site-directed mutagenesis on the nucleic acid, or induced by mutagenic treatment, or may be naturally occurring, i.e. isolated from a natural source. In some embodiments, a progenitor plant cell, tissue, seed or plant may be subjected to mutagenesis to produce single or multiple mutations, such as nucleotide substitutions, deletions, insertions and/or codon modification. Preferred wheat plants and grain of the invention comprise at least one introduced SSIIa gene mutation, more preferably two or more introduced SSIIa gene mutations, and may comprise no mutations from a natural source i.e. all of the mutant SSIIa alleles in the plant were obtained by synthetic means or by mutagenic treatment. As used herein, an “induced mutation” or “introduced mutation” is an artificially induced genetic variation which may be the result of chemical, radiation or biologically-based mutagenesis, for example transposon or T-DNA insertion, or an endonuclease induced mutation.

Mutagenesis can be achieved by chemical or radiation means, for example EMS or sodium azide (Zwar and Chandler, 1995) treatment of seed, or gamma irradiation, well know in the art. Chemical mutagenesis tends to favour nucleotide substitutions rather than deletions. Heavy ion beam (HIB) irradiation is known as an effective technique for mutation breeding to produce new plant cultivars, see for example Hayashi et al., 2007 and Kazama et al, 2008. Ion beam irradiation has two physical factors, the dose (gy) and LET (linear energy transfer, keV/um) for biological effects that determine the amount of DNA damage and the size of DNA deletion, and these can be adjusted according to the desired extent of mutagenesis. HIB generates a collection of mutants, many of them comprising deletions that may be screened for mutations in specific SSIIa genes. Mutants which are identified may be backcrossed with non-mutated wheat plants as recurrent parents in order to remove and therefore reduce the effect of unlinked mutations in the mutagenised genome.

Isolation of mutants may be achieved by screening mutagenised plants or seed. For example, a mutagenized population of wheat may be screened directly for the SSIIa genotype or indirectly by screening for a phenotype that results from mutations in the SSIIa genes. Screening directly for the genotype preferably includes assaying for the presence of mutations in the SSIIa genes, which may be observed in PCR assays by the absence of specific SSIIa markers as expected when some of the genes are deleted, or heteroduplex based assays as in TILLING. Screening is preferably based on nucleotide sequencing which is often based on pools of candidate mutants. Screening for the phenotype may comprise screening for a loss or reduction in amount of one or more SSIIa polypeptides by ELISA or affinity chromatography, or altered starch phenotypes in the grain starch. In hexaploid wheat, screening is preferably done in a genotype that already lacks one or two of the SSIIa activities, for example in a wheat plant already mutant in the SSIIa genes of two of the three genomes, so that a mutant further lacking the functional activity is sought. Affinity chromatography may be carried out to distinguish the SSIIa-A, SSIIa-B and SSIIa-D polypeptides. Large populations of mutagenised seeds (thousands or tens of thousands of seeds) may be screened for high amylose phenotypes using near infra-red spectroscopy (NIR). By these means, high throughput screening is readily achievable and allows the isolation of mutants at a frequency of approximately one per several hundred seeds.

Plants and seeds of the invention can be produced using the process known as TILLING (Targeting Induced Local Lesions IN Genomes), in that one or more of the mutations in the wheat plants or grain may be produced by this method. In a first step, introduced mutations such as novel single base pair changes are induced in a population of plants by treating seeds or pollen with a chemical or radiation mutagen, and then advancing plants to a generation where mutations will be stably inherited, typically an M2 generation where homozygous mutants may be identified. DNA is extracted, and seeds are stored from all members of the population to create a resource that can be accessed repeatedly over time. For a TILLING assay, PCR primers are designed to specifically amplify a single gene target of interest. Next, dye-labelled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, which recognizes and cleaves mismatched DNA, or the use of High Resolution Melting, is used to discovering novel SNPs within a TILLING population. For example, see Botticella et al., 2011.

Using this approach, many thousands of plants can be screened to identify any individual with a single base change or small insertions or deletions (1-30 bp) in any gene or specific region of the genome. Genomic fragments being assayed can range in size anywhere from 0.3 to 1.6 kb. At 8-fold pooling and amplifying 1.4 kb fragments with 96 lanes per assay, this combination allows up to a million base pairs of genomic DNA to be screened per single assay, making TILLING a high-throughput technique. TILLING is further described in Slade and Knauf, 2005, and Henikoff et al., 2004.

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004). Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping mutations in a single step. In this way, sequencing of the mutant gene is simple and efficient.

As used herein the term “biological agents” means agent useful in producing site-specific mutants and includes enzymes that induce double stranded breaks in DNA that stimulate endogenous repair mechanisms. These include endonucleases, zinc finger nucleases, TAL effector proteins, transposases, site-specific recombinases and are preferably CRISPR endonucleases. Zinc finger nucleases (ZFNs), for example, facilitate site-specific cleavage within a selected gene within a genome allowing endogenous or other end-joining repair mechanisms to introduce deletions or insertions to repair the gap. Zinc finger nuclease technology is reviewed in Le Provost et al., 2009, See also Durai et al., 2005 and Liu et al., 2010.

Clustered regularly interspaced short palindromic repeats (CRISPR) are segments of prokaryotic DNA containing short repetitions of base sequences. Each repetition is followed by short segments of “spacer DNA” from previous exposures to a bacteriophage virus or plasmid. The CRISPR/Cas system is a prokaryotic immune system that confers resistance to foreign genetic elements such as those present within plasmids and phages, and provides a form of acquired immunity. CRISPR spacers recognize and cut these exogenous genetic elements in a manner analogous to RNA interference in eukaryotic organisms. CRISPRs are found in approximately 40% of sequenced bacterial genomes and 90% of sequenced archaea.

By delivering the Cas9 nuclease and appropriate guide RNAs into a cell, the cell's genome can be cut at a desired location, allowing existing genes to be removed and/or new ones added. CRISPRs have been used in concert with specific endonuclease enzymes for genome editing and gene regulation in various species. Further information regarding CRISPR can be found in WO 2013/188638, WO 2014/093622 and Doudna et al., (2014).

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing. Further information regarding TALEN can be found in Boch (2011); Juong et al., (2013) and Sune et al., (2013).

Identified mutations may then be introduced into desirable genetic backgrounds by crossing the mutant with a plant of the desired genetic background and performing a suitable number of backcrosses to cross out the originally undesired parent background. See, for example, Example 3 herein.

In some embodiments, mutations are null mutations such as nonsense mutations, frameshift mutations, deletions, insertional mutations or splice-site variants which completely inactivate the gene. Nucleotide insertional derivatives include 5′ and 3′ terminal fusions as well as intra-sequence insertions of single or multiple nucleotides.

Insertional nucleotide sequence variants are those in which one or more nucleotides are introduced into a site in the nucleotide sequence, either at a predetermined site as is possible with zinc finger nucleases (ZFN), CRISPR nucleases or other homologous recombination methods, or by random insertion with suitable screening of the resulting product.

Deletional variants are characterised by the removal of one or more nucleotides from the sequence. In an embodiment, a mutant gene has only a single insertion or deletion of a sequence of nucleotides relative to the wild-type gene. The deletion may be extensive enough to include one or more exons or introns, both exons and introns, an intron-exon boundary, a part of the promoter, the translational start site, or even the entire gene. Deletions may extend far enough to include at least part of, or the whole of, an SSIIa gene and one or more adjacent genes on the A, B or D genome. Insertions or deletions within the exons of the protein coding region of a gene which insert or delete a number of nucleotides which is not an exact multiple of three, thereby causing a change in the reading frame during translation, almost always abolish activity of the mutant gene comprising such insertion or deletion; such mutations are null mutations. Insertions or deletions within the exons of the protein coding region of a gene which insert or delete a number of nucleotides which is an exact multiple of three may or may not abolish activity of the gene comprising such insertion or deletion. In the case of a deletion of an exact multiple of three nucleotides, the deletion would be expected to inactivate the encoded polypeptide if the deleted nucleotides encode a highly conserved amino acid. Enzyme assays or phenotypic assays can be used to determine if insertion or deletion mutations are null mutations.

Substitutional nucleotide variants are those in which at least one nucleotide in the sequence has been removed and a different nucleotide inserted in its place. In some embodiments, the number of nucleotides affected by substitutions in a mutant gene relative to the wild-type gene is a maximum of ten nucleotides, more preferably a maximum of 9, 8, 7, 6, 5, 4, 3, or 2, or most preferably only one nucleotide. Substitutions may be “silent” in that the nucleotide substitution does not change the amino acid defined by the codon. Nucleotide substitutions may reduce the translation efficiency and thereby reduce the expression level of the affected SSIIa gene, for example by reducing the mRNA stability or, if near an exon-intron splice boundary, alter the splicing efficiency. Silent substitutions that do not alter the translation efficiency of an SSIIa gene are not expected to alter the activity of the gene and are therefore regarded herein as non-mutant, i.e. such genes are active variants and not encompassed in “mutant gene”. Alternatively, the nucleotide substitution(s) may change the encoded amino acid sequence and thereby alter the activity of the encoded enzyme, particularly if conserved amino acids are substituted for another amino acid which is quite different i.e. a non-conservative substitution. For conservative substitutions, see Table 3. Conserved amino acids in wheat SSIIa polypeptides may be identified by aligning SSIIa amino acid sequences from different species, for example aligning SEQ ID NO:1 with an Arabidopsis thaliana SSII and determining which amino acids are in common.

The term “mutation” as used herein does not include silent nucleotide substitutions which do not affect the activity of the gene, and therefore includes only alterations in the gene sequence which affect the gene activity. The term “polymorphism” refers to any change in the nucleotide sequence of the gene including such silent nucleotide substitutions. Screening methods may first involve screening for polymorphisms and secondly for mutations within a group of polymorphic variants. Mutations include deletions of all or part of a gene, insertions such as an insertion into an exon of a gene, and nucleotide substitutions, and any combination thereof.

The terms “plant(s)” and “wheat plant(s)” as used herein as a noun generally refer to whole plants, but when “plant” or “wheat” is used as an adjective, the terms refer to any substance which is present in, obtained from, derived from, or related to a plant or a wheat plant, such as for example, plant organs (e.g. leaves, stems, roots, flowers), single cells (e.g. pollen), seeds or grains, plant cells including for example tissue cultured cells, products produced from the plant such as “wheat flour”, “wheat grain”, “wheat starch”, “wheat starch granules” and the like. Plantlets and germinated grain from which roots and shoots have emerged are also included within the meaning of “plant”. The term “plant parts” as used herein refers to one or more plant tissues or organs which are obtained from a whole plant, preferably a wheat plant. Plant parts as used herein comprise plant cells. Plant parts include vegetative structures (for example, leaves including the leaf sheath and leaf blade, stems including the internodes), roots, tillers, floral organs/structures such as the spike (also called the ear or head), pollen, ovules, seed (including embryo, endosperm, and seed coat), plant tissue (for example, vascular tissue, ground tissue, and the like), cells and progeny of the same. The term “plant cell” as used herein refers to a cell obtained from a plant or in a plant, preferably a wheat plant, and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, pollen, and various forms of aggregations of plant cells in culture, such as calli. Plant tissues in or from seeds such as wheat grain are the seed coat, endosperm, scutellum, aleurone layer and embryo. Each of the wheat plant tissues or organs and wheat cells comprise genetic material (nucleic acid) of the wheat plant from which is obtained.

Cereals as used herein means plants or grain of the monocotyledonous families Poaceae or Graminae which are cultivated for the edible components of their grain, and includes wheat, barley, maize, oats, rye, rice, sorghum, triticale, millet, buckwheat. Preferably, the cereal plant or grain is a wheat plant or grain. In a further preferred embodiment, the wheat cell, plant or grain, or products derived therefrom, of the invention is of the species Triticum aestivum.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. The plants and grain of the invention are of the hexaploid species T. aestivum, and do not include the tetraploid species T. durum, also referred to as durum wheat or Triticum turgidum ssp. durum. Diploid progenitors of T. aestivum are thought to be T. uartu, T. monococcum or T. boeoticum for the A genome, Aegilops speltoides for the B genome, and T. tauschii (also known as Aegilops squarrosa or Aegilops tauschii) for the D genome. Preferably the T. aestivum plant of the invention is suitable for commercial production of grain, having suitable agronomic characteristics which are known to those skilled in the art. Most preferably the wheat is Triticum aestivum ssp. aestivum, herein also referred to as “breadwheat”.

Aspects of the invention provide methods of planting and harvesting wheat grain of the invention, and methods of producing bins of wheat grain of the invention. For instance, after the ground is prepared by ploughing and/or certain other methods, the seeds are typically planted by sowing by drilling furrows and planting the seeds in rows. To prevent scattering of the grain produced upon plant maturity, wheat may be harvested before it is fully ripe, but is typically harvested when the plants show complete loss of green colour. There are several steps in harvesting: cutting, or reaping, the stalks; threshing and winnowing, to separate the grain from the spikes, glumes, and other chaff; sifting and sorting the grain; typically done by a combine harvester, then loading the grain into trucks. In some embodiments harvested wheat grain may be stored in dry, well-ventilated buildings that keep out insect pests. In some embodiments, harvested wheat grain may be stored for a short time in bins or granaries. The wheat grain may then by hauled to country elevators, tall structures where the grain is dried and stored until it is sold or shipped to terminal elevators. Therefore, embodiments of the invention provide a process of producing bins of wheat grain comprising: a) reaping wheat stalks comprising wheat grain as defined herein; b) threshing and/or winnowing the stalks to separate the grain from the chaff; and c) sifting and/or sorting the grain separated in step b), and loading the sifted and/or sorted grain into bins, thereby producing bins of wheat grain.

The wheat plants and grain of the invention have uses other than uses for food or animal feed, for example uses in research or breeding. In seed propagated crops such as wheat, the plants can be self-crossed to produce a plant which is homozygous for the desired genes, or haploid tissues such as developing germ cells can be induced to double the chromosome complement to produce a homozygous plant. The inbred wheat plant of the invention thereby produces grain containing the combination of mutant SSIIa alleles which are homozygous. The grain can be grown to produce plants that would have the selected phenotype such as, for example, high amylose content in its starch.

The wheat plants of the invention may be crossed with plants containing a more desirable genetic background, and therefore the invention includes the transfer of the reduced SSIIa trait to other genetic backgrounds. As used herein, “crossing” or “cross” refers to the process by which the pollen of a flower on one plant is applied (artificially or naturally) to the stigma of a flower of another plant. After the initial crossing, a suitable number of backcrosses may be carried out to remove a less desirable background. SSIIa allele-specific PCR-based markers such as those described herein may be used to screen for or identify progeny plants or grain with the desired combination of alleles, thereby tracking the presence of the alleles in the breeding program. The desired genetic background may include a suitable combination of genes providing commercial yield and other characteristics such as agronomic performance or abiotic stress resistance. The genetic background might also include other altered starch biosynthesis or modification genes, for example null alleles of SS/Ha genes or favourable alleles of GBSS genes. The genetic background may comprise one or more transgenes such as, for example, a gene that confers tolerance to an herbicide such as glyphosate.

The desired genetic background of the wheat plant will include considerations of agronomic yield and other characteristics. Such characteristics might include whether it is desired to have a winter or spring types, agronomic performance, disease resistance and abiotic stress resistance. For Australian use, one might want to cross the altered starch trait of the wheat plant of the invention into wheat cultivars such as Baxter, Kennedy, Janz, Frame, Rosella, Cadoux, Diamondbird or other commonly grown varieties. Other varieties will be suited for other growing regions. It is preferred that the wheat plant of the invention provide a grain yield (tonnes/hectare) of at least 50% relative to the yield of the corresponding wild-type variety in at least some growing regions, more preferably at least 60% or at least 70%, or at least 80% or at least 90%, relative to a wild-type variety having about the same genetic background, grown under the same conditions. In an embodiment, the yield of grain is less than 90% relative to a wild-type variety having about the same genetic background, grown under the same conditions. The yield can readily be measured in controlled field trials, or in simulated field trials in the greenhouse, preferably in the field.

Marker assisted selection is a well recognised method of selecting for heterozygous plants obtained when backcrossing with a recurrent parent in a classical breeding program. The population of plants in each backcross generation will be heterozygous for the gene(s) of interest normally present in a 1:1 ratio in a backcross population, and a molecular marker linked to a gene can be used to distinguish the two alleles of the gene. The presence of two markers can be assayed, one for a mutant allele and the other for the wild-type allele. By extracting DNA from, for example, young shoots and testing with a specific marker for the introgressed desirable trait, early selection of plants for further backcrossing is made whilst energy and resources are concentrated on fewer plants. Procedures such as crossing wheat plants, self-fertilising wheat plants or marker-assisted selection are standard procedures and well known in the art. Transferring alleles from tetraploid wheat such as durum wheat to a hexaploid, or other forms of hybridisation, is more difficult but is also known in the art.

To identify the desired phenotypic characteristic, wheat plants that contain a combination of mutant ssIIa or other desired genes are typically compared to a control plant. When evaluating a phenotypic characteristic associated with mutant ssIIa genes such as amylose content in the grain starch, or total fibre content or grain weight or yield, the plants to be tested and control plants are grown under growth chamber, glasshouse or preferably field conditions, under the same conditions (temperature, soil, moisture supply, fertiliser supply, season etc). Identification of a particular phenotypic trait and comparison to controls is based on routine statistical analysis and scoring. Expression of genes or enzyme activity are compared to growth, development and yield parameters which include one or more of germination rate, seedling vigour including seedling emergence, plant morphology, colour, number, size, dimensions, dry and wet weight, ripening, above- and below-ground biomass ratios, and timing, rates and duration of various stages of growth through senescence, including vegetative growth, fruiting, flowering, grain yield and dormancy, harvest index, and soluble carbohydrate content including sucrose, glucose, fructose and starch levels as well as endogenous starch levels. In some embodiments, the wheat plants of the invention differ from wild-type plants in one or more of these parameters by less than 50%, more preferably less than 40%, less than 30%, less than 20%, less than 15%, less than 10%, less than 5%, less than 2% or less than 1% when grown under the same conditions. Preferably, the plant or grain of the invention is about the same as the wild-type plant or grain for one or more of these parameters.

As used herein, the term “linked” or “genetically linked” refers to a marker locus and a second locus being sufficiently close on a chromosome that they will be inherited together in more than 50% of meioses, e.g., not randomly. This definition includes the situation where the marker locus and second locus form part of the same gene. Furthermore, this definition includes the situation where the marker locus comprises a polymorphism that is responsible for the trait of interest, in which case the polymorphism will be 100% linked to the phenotype. Thus, the percent of recombination observed between the loci per generation (calculated as centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM or 2cM apart and most preferably essentially 0 cM apart.

As used herein, the “other genetic markers” may be any molecules which are linked to a desired trait in the wheat plants of the invention. Such markers are well known to those skilled in the art and include molecular markers linked to genes determining traits such disease resistance, yield, plant morphology, grain quality, other dormancy traits such as grain colour, gibberellic acid content in the seed, plant height, flour colour and the like. Examples of such genes are stem-rust resistance genes Sr2 or Sr38, the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance (Eagles et al., 2001; Langridge et al., 2001; Sharp et al., 2001).

The wheat plants, wheat plant parts and products therefrom of the invention are preferably non-transgenic for genes that inhibit expression of an SSIIa gene and/or an SBEIIa gene, i.e. they do not comprise a transgene encoding an RNA molecule that reduces expression of an endogenous SSIIa gene, although in this embodiment they may comprise other transgenes, eg. herbicide tolerance genes such as glyphosate tolerance. More preferably, the wheat plant, grain and products therefrom are non-transgenic, i.e. they do not contain any transgene, which is preferred in some markets. Such products are also described herein as “non-transformed” products. Such non-transgenic plants and grain comprise the multiple mutant SSIIa alleles as described herein, such as those produced after mutagenesis.

The terms “transgenic plant” and “transgenic wheat plant” as used herein refer to a plant that contains a genetic construct (“transgene”) not found in a wild-type plant of the same species, variety or cultivar. That is, transgenic plants (transformed plants) contain genetic material that they did not contain prior to the transformation. A “transgene” or “genetic construct” as referred to herein has the normal meaning in the art of biotechnology and refers to a genetic sequence which has been produced or altered by recombinant DNA or RNA technology. If present in a plant cell, the transgene had been introduced into the plant cell or a progenitor cell by a human. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is typically stably integrated into the genome of the plant. The introduced genetic material may comprise sequences that naturally occur in the same species but in a rearranged order or in a different arrangement of elements, for example an antisense sequence or a sequence expressing an inhibitory double-stranded RNA. Plants containing such sequences are included herein in “transgenic plants”. Transgenic plants as defined herein include all progeny of an initial transformed and regenerated plant (TO plant) which has been genetically modified using recombinant techniques, where the progeny comprise the transgene. Such progeny may be obtained by self-fertilisation of the primary transgenic plant or by crossing such plants with another plant of the same species. In an embodiment, the transgenic plants are homozygous for each and every gene that has been introduced (transgene) so that their progeny do not segregate for the desired phenotype. Transgenic plant parts include all parts and cells of said plants which comprise the transgene such as, for example, seeds, cultured tissues, callus and protoplasts.

A “non-transgenic plant”, preferably a non-transgenic wheat plant, is one which has not been genetically modified by the introduction of genetic material by recombinant DNA techniques. The presence in a plant or grain of deletions of part of a gene as generated by site-specific endonucleases such as ZFN, TAL effectors of CRISPR type nucleases, followed by non-homologous end-joining repair in the plant cell, and progeny thereof are included herein as “non-transgenic”. As used herein, “progeny” includes all offspring from a wheat plant, both the immediate and subsequent generations, and both plants and seed (grain). Progeny include the seeds and plants obtained after self-fertilisation (“selfing”) and the grain and plants resulting from a cross between two parental plants, such as the F1 offspring (first generation), F2, F3, F4 etc being the offspring from the second etc generations after selfing of the F1 plants.

As used herein, the term “corresponding non-transgenic plant” refers to a plant which is the same or similar in most characteristics, preferably isogenic or near-isogenic relative to the transgenic plant, but without the transgene of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant.

“Wild-type”, as used herein, refers to a cell, tissue, plant or plant part, preferably a Triticum aestivum plant, plant part or grain, which has not been modified according to the invention. Such a wild-type plant or grain is at least wild-type for its SSIIa genes. “Corresponding wild-type” refers to a wild-type cell, tissue, plant, plant part or plant product which is suitable as a comparison to the cell, tissue, plant, plant part or plant product of the invention, as readily understood in the art. Generally, the corresponding wild-type is from a genetically similar, preferably isogenic, wheat plant or plant part to the plant or plant part of the invention but not having the ssIIa gene mutations. Wild-type cells, tissue or plants are known in the art and may be used as controls to compare gene or polypeptide sequences, in particular SSIIa gene sequences, levels of expression of an SSIIa gene or the extent and nature of trait modification with cells, tissue or plants modified as described herein. As used herein, “wild-type wheat grain” means a corresponding non-mutagenized, non-transgenic wheat grain. Specific wild-type wheat grains as used herein include but are not limited to Sunstate, Chara and Cadoux. The Sunstate wheat cultivar is described in Ellison et al., (1994).

Any of several methods may be employed to determine the presence of a transgene in a transformed plant. For example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers that will distinguish the transformed and non-transformed plants. An alternative method to confirm a positive transformant is by Southern blot hybridization, well known in the art. Wheat plants which are transformed may also be identified i.e. distinguished from non-transformed or wild-type wheat plants by their phenotype, for example conferred by the presence of a selectable marker gene, or by immunoassays that detect or quantify the expression of an enzyme encoded by the transgene, or any other phenotype conferred by the transgene.

The wheat plants, preferably of the species Triticum aestivum, of the present invention may be grown or harvested for grain, primarily for use as food for human consumption or as animal feed, or for fermentation or industrial feedstock production such as ethanol production, among other uses. Alternatively, the green, aerial parts of wheat plants may be used directly as feed for animals, either directly by grazing in the field or after harvesting. The straw and chaff can also be used as feed or for non-food use. The plant of the present invention is preferably useful for food production and in particular for commercial food production for human consumption. Such food production might include the making of flour, dough, semolina or other products from the grain such as starch granules or isolated starch that might be an ingredient in commercial food production.

As used herein, the term “grain” generally refers to mature, harvested seed (also called the kernel) of a plant but can also refer to grain after imbibition or germination, according to the context. Grain includes the mature kernels produced by growers for purposes other than growing further plants. Mature cereal grain such as wheat commonly has a moisture content of less than about 18-20%, typically about 8-10% moisture. As used herein, the term “seed” includes harvested seed but also includes seed which is developing in the plant post anthesis and mature seed comprised in the plant prior to harvest. The parts of the grain include the testa (seedcoat), the pericarp (fruit coat), the aleurone layer, the starchy endosperm and the embryo (germ) which is made up of the scutellum, the plumule (shoot) and radicle (primary root). The combined testa, pericarp and aleurone layer are commonly referred to as the “bran”, which can be removed from the grain by milling, which may also comprise the germ. The scutellum is the region that secretes some of the enzymes involved in germination and absorbs the soluble sugars from the breakdown of starch in the endosperm for growth of the seedling after germination. The aleurone which surrounds the starchy endosperm also secretes enzymes during germination.

As used herein, “germination” refers to the emergence of the root tip (radicle) from the seed coat after imbibition. The radicle usually emerges first and then the plumule. “Germination rate” refers to the percentage of seeds in a population which have germinated over a period of time, for example 7 or 10 days, after imbibition. Germination rates can be calculated using techniques known in the art. For example, a population of seeds, typically at least 100 grains, can be assessed daily over several days to determine the germination percentage over time. With regard to grain of the present invention, as used herein the term “germination rate which is substantially the same” means that the germination rate of the grain is at least 90%, that of corresponding wild-type grain. In an embodiment, the grain of the invention has a germination rate of between about 70% and about 100% relative to wild-type grain, preferably between about 90% and about 100% relative to wild-type grain. When measuring germination, the wheat grain of the invention and the wild-type grain used as a control should have been grown under the same conditions and stored under the same conditions for about the same length of time.

The invention also provides food ingredients such as flour, preferably wholemeal, bran and other products produced from the grain. These may be unprocessed or processed, for example by heat treatment, fractionation or bleaching.

The grain of the invention can be processed to produce a food ingredient or a food or non-food product using any technique known in the art. In one embodiment, the food ingredient is flour such as, for example, wholemeal (wholemeal flour) or white flour. As used herein, “flour” is a milled product from the grain which has been milled to a powder. The powder is commonly fractionated by sieving, sifting, centrifugation or other methods known in the art, and may be further refined, heat treated and/or bleached. Refined flour, or “white flour” as referred to herein refers to flour which has been enriched for the endosperm-derived part of the milled powder relative to wholemeal, performed by removing at least some of the bran and germ components of the milled powder. The Food and Drug Administration (FDA) requires flour to meet certain particle size standards in order to be included in the category of refined flour. According to the FDA, the particle size of refined flour is described as flour in which not less than 98% passes through a cloth having openings not larger than those of woven wire cloth designated “212 micrometers (U.S. Wire 70)”.

As used herein, the term “wholemeal”, also called wholemeal flour or whole grain flour, is a milled flour which was made from essentially 100% of the grain and which includes a refined flour constituent (refined flour or refined flour) and a coarse fraction (an ultrafine-milled coarse fraction). The coarse fraction includes at least one of bran and germ, typically both. The germ is an embryonic plant found within the grain kernel, comprising the embryo and scutellum. The germ includes lipids, fibre, vitamins, protein, minerals and phytonutrients, such as flavonoids, at levels higher than in the mature endosperm of the grain. The bran includes several cell layers including the pericarp (fruit coat) and testa (seed coat) and also has a significant amount of lipids, fibre, vitamins, protein, minerals and phytonutrients, such as flavonoids. The aleurone layer, while technically considered part of the endosperm of the mature grain, exhibits many of the same characteristics as the bran and therefore is typically removed with the bran and germ during the milling and/or sieving process. The aleurone layer also includes lipids, fibre, vitamins, protein, minerals and phytonutrients, such as flavonoids and ferulic acid.

Further, the coarse fraction may be blended with the refined flour constituent. Preferably, the coarse fraction is homogenously blended with the refined flour constituent. The coarse fraction may be mixed with the refined flour constituent to form the wholemeal, thus providing a wholemeal with increased nutritional value, fibre content, and antioxidant capacity as compared to refined flour. For example, the coarse fraction or wholemeal may be used in various amounts to replace refined flour in baked goods, snack products, and food products. The wholemeal of the present invention (i.e. ultrafine-milled whole grain flour) may also be marketed directly to consumers for use in their homemade baked products. In an exemplary embodiment, a granulation profile of the wholemeal is such that 98% of particles by weight of the wholemeal are less than 212 micrometers in diameter.

In further embodiments, enzymes found within the bran and germ of the wholemeal and/or coarse fraction are inactivated in order to stabilize the wholemeal and/or coarse fraction. It is contemplated by the present invention that “inactivated” may also mean inhibited, denatured, or the like. Stabilization is a process that uses steam, heat, radiation, or other treatments to inactivate the enzymes found in the bran and germ layer. In the absence of stabilization, naturally occurring enzymes in the bran and germ catalyze changes to compounds in the flour, which may adversely affecting the cooking characteristics of the flour and the shelf life. Inactivated enzymes do not catalyze changes to compounds found in the flour, therefore, flour that has been stabilized retains its cooking characteristics and has a longer shelf life. For example, the present invention may implement a two-stream milling technique to grind the coarse fraction. Once the coarse fraction is separated and stabilized, the coarse fraction is then ground through a grinder, preferably a gap mill, to form a coarse fraction having a particle size distribution less than or equal to about 500 micrometers. After sifting, any ground coarse fraction having a particle size greater than 500 micrometers may be returned to the process for further milling.

In additional embodiments, the flour, wholemeal or the coarse fraction may be a component of a food product, for example, may be used as an ingredient in food production. The food product may be, for example, a bagel, a biscuit, a bread, a bun, a croissant, a dumpling, a muffin such as an English muffin, a pita bread, a quickbread, a refrigerated or frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, to mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In other embodiments, the flour, wholemeal or coarse fraction may be a component of a nutritional supplement. For instance, the nutritional supplement may be a product that is added to the diet containing one or more ingredients, typically including: vitamins, minerals, lipids such as omega-3 fatty acids, amino acids; enzymes, antioxidants such as lutein, herbs, spices, probiotics, extracts, prebiotics and fibre. The flour, wholemeal or coarse fraction of the present invention includes vitamins, minerals, amino acids, enzymes, and fibre. For instance, the coarse fraction contains a concentrated amount of dietary fibre as well as other essential nutrients, such as B-vitamins, selenium, chromium, manganese, magnesium, and antioxidants, which are essential for a healthy diet. For example, 15 grams of the coarse fraction of the present invention delivers 33% of an individual's daily recommend consumption of fibre. Further, 9 grams is all that is needed to deliver 20% of an individual's daily recommend consumption of fibre. Thus, the coarse fraction is an excellent supplemental source for consumption of an individual's fibre requirement.

In additional embodiments, the wholemeal or coarse fraction may be a fibre supplement or a component thereof. Many current fibre supplements such as psyllium husks, cellulose derivatives and hydrolyzed guar gum have limited nutritional value beyond their fibre content. Additionally, many fibre supplements have an undesirable texture and poor taste. Therefore, in an embodiment, the food ingredients of the invention lack fibre supplements derived from sources other than wheat grain. Supplements made from the wholemeal or coarse fraction of the wheat grain thereby deliver fibre as well as protein, and antioxidants. The fibre supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fibre supplement in the form of a flavored shake or malt type beverage, this embodiment may be particularly attractive as a fibre supplement for children.

In an additional embodiment, a milling and blending process may be used to make a multi-grain flour or a multi-grain coarse fraction. For example, bran and germ from one type of grain such as grain of the invention may be ground and blended with ground endosperm or whole grain flour of another type of wheat or other cereal. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of grains such as wheat to make one flour.

The flour or wholemeal of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Any sifter known in the art for sifting a ground particle may be used. Material passing through the screen of the sifter is the whole grain flour of the present invention and requires no further processing. Material that remains on the screen is referred to as a second fraction. The second fraction requires additional particle reduction. Thus, this second fraction may be conveyed to a second passage grinder. After grinding, the second fraction may be conveyed to a second sifter.

It is contemplated that the flour, wholemeal, coarse fraction and/or grain products of the present invention may be modified or enhanced by way of numerous other processes such as: fermentation, instantizing, extrusion, encapsulation, toasting, roasting, or the like. The flour and wholemeal of the invention comprise the genetic material, including the DNA, of the wheat grain from which they were derived, as do the food products produced therefrom. See for example, Tilley (2004) and Bryan et al., (1998).

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:
(i) providing wheat grain of the invention,
(ii) steeping said grain,
(iii) germinating the steeped grains under predetermined conditions and
(iv) drying said germinated grains.

Malt may be prepared using only grain of the invention or in mixtures comprising other grains. Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

In general, the first step in the wort production process is the milling of malt in order that water may gain access to grain particles in the mashing phase, which is fundamentally an extension of the malting process with enzymatic depolymerization of substrates. During mashing, milled malt is incubated with a liquid fraction such as water. The temperature is either kept constant (isothermal mashing) or gradually increased. In either case, soluble substances produced in malting and mashing are extracted into said liquid fraction before it is separated by filtration into wort and residual solid particles denoted spent grains. The wort composition may also be prepared by incubating wheat grain of the invention or parts thereof with one or more suitable enzyme, such as enzyme compositions or enzyme mixture compositions, for example Ultraflo or Cereflo (Novozymes). The wort composition may also be prepared using a mixture of malt and unmalted plants or parts thereof, optionally adding one or more suitable enzymes during said preparation.

The starch of the flour or wholemeal when incorporated in food products provides modified digestive properties, for example the food ingredient comprises increased resistant starch relative to a corresponding food ingredient produced from wild-type wheat grain, such as including between 1% to 20%, 2% to 18%, 3% to 18% or 5% to 15% resistant starch, and a decreased Glycaemic Index (GI), such as a reduction by at least 5 units, preferably between 5 and 25 units. This may be in combination with an increased total fibre content, for example, of 15% to 30% by weight in the food ingredient.

Carbohydrates, compounds comprising one or more saccharide units comprised of carbon, hydrogen and oxygen, can be classified according to the number and composition of the monosaccharide units making up the carbohydrate. These include polysaccharides (>10 monosaccharide units), oligosaccharides (3-10 monosaccharide units), disaccharides and monosaccharides including glucose, fructose, xylulose and arabinose. Carbohydrates make up more than 65% by weight of the mature wild-type wheat grain, including 65-75% starch and about 10% cell wall polysaccharides such as cellulose, arabinoxylan and BG.

Starch is the major storage carbohydrate in most plants, including cereals such as wheat. “Starch” is defined herein as polysaccharide composed of glucopyranose units polymerized through α-1,4 linkages and either no or some α-1,6 linkages. Starch is synthesized in the amyloplasts and formed and stored in granules in the developing storage organ such as grain; it is referred to herein as “storage starch” or “grain starch” or “starch of the grain”. In cereal grains including Triticum aestivum, the great majority of the storage starch is deposited in the endosperm as starch granules. Starch is synthesized and deposited within amyloplasts during grain development, in particular the grain filling phase of plant growth, and forms discrete crystalline structures termed starch granules. In wild-type Triticum aestivum, the starch granules are of two size classes, namely larger, ellipsoidal granules ranging from 10-40 μm in diameter (A-type granules) and smaller, spherical granules from 1-10 μm in diameter (B-type granules).

The molecules of starch are classified as belonging to two component fractions, known as amylose and amylopectin, which are distinguished on the basis of their degree of polymerization (DP) and the ratio of α-1,6 to α-1,4 linkages in the polymers. Amylose comprises almost entirely linear α-1,4 linked glucosyl chains which may have either no or a few glucan chains joined by an α-1,6 bond to other α-1,4 linked chains and has a molecular weight of 10⁴ to 10⁵ daltons. The term “amylose” is defined herein as including essentially linear molecules of α-1,4 linked glucosidic (glucopyranose) units, sometimes referred to as “true amylose”, and amylose-like long-chain starch which is sometimes referred to as “intermediate material” or “amylose-like amylopectin” which appears as iodine-binding material in an iodometric assay along with true amylose (Takeda et al., 1993; Fergason, 1994). The linear molecules in true amylose typically have a DP of between 500 and 5000 and contain less than 1% α-1,6 linkages. Recent studies have shown that about 0.1% of α-1,6-glycosidic branching sites may occur in amylose, therefore it is described as “essentially linear”. In this context, the percentage (%) refers to the number of α-1,6-glycosidic bonds relative to the total number of glycosidic bonds, being the sum of α-1,4-glycosidic bonds and α-1,6-glycosidic bonds. Granule bound starch synthase (GBSS) is the main enzyme involved in synthesis of amylose.

Amylopectin is a relatively highly branched glucan polymer in which α-1,4-linked glucosyl chains with 3 to 60 glucosyl units are connected by α-1,6 linkages, so that approximately 4-6% of the total number of glucosyl linkages are α-1,6 linkages. Therefore, amylopectin is a much larger molecule with a DP ranging from 5000 to 500,000 and is more highly branched than amylose. Amylose has a helical conformation with a molecular weight of about 10⁴ to about 10⁶ Daltons while amylopectin has a molecular weight of about 10⁷ to about 10⁸ Daltons. These two types of starch can readily be distinguished or separated by methods well known in the art, for example by size exclusion chromatography or by their different binding affinity for iodine. Amylose is digested more slowly by α-amylases in the small intestine than amylopectin, the latter having multiple sites for enzymatic hydrolysis because of its highly branched structure.

Starch from wild-type Triticum aestivum grain typically comprises 20%-30% of amylose and about 70%-80% of amylopectin as measured by an iodometric method, whereas the starch of the grain of the invention has an amylose content of about 45% to about 70% on a weight basis. The amylose content may be between 45% and 70%, in some embodiments between 45% and 65%, or about 50%, about 55%, about 60% or about 65%. The higher the level, the more preferred is the grain. The proportion of amylose in the starch as defined herein is on a weight/weight (w/w) basis, i.e. the weight of amylose as a percentage of the weight of total starch extractable from the grain, with respect to the starch prior to any fractionation into amylose and amylopectin fractions. The terms “proportion of amylose in the starch” and “amylose content” when used herein in the context of the grain, flour or other product of the invention are essentially interchangeable terms. Amylose content may be determined by any of the methods known in the art including size exclusion high-performance liquid chromatography (HPLC), for example in 90% (w/v) DMSO, concanavalin A methods (Megazyme Int, Ireland), or preferably by an iodometric method, for example as described in Example 1. The HPLC method may involve debranching of the starch (Batey and Curtin, 1996) or not involve debranching. It will be appreciated that methods such as the HPLC method of Batey and Curtin, 1996 which assay only the “true amylose” may underestimate the amylose content as defined herein. Methods such as HPLC or gel permeation chromatography depend on fractionation of the starch into the amylose and amylopectin fractions, while iodometric methods depend on differential iodine binding and therefore do not require fractionation.

From the grain weight and amylose content, the amount of amylose deposited per grain can be calculated and compared for test and control lines.

Starch is readily isolated from wheat grain using standard methods, for example the method of Schulman and Kammiovirta, 1991. On an industrial scale, wet or dry milling can be used. Starch granule size is important in the starch processing industry where there may be separation of the larger A granules from the smaller B granules.

Wild-type wheat grown commercially has a starch content in the grain which is usually in the range 55-75%, depending somewhat on the cultivar grown. In comparison, the grain of the invention has a starch content of about 25% to about 70%, so in most embodiments its starch content is reduced relative to corresponding wild-type grain. In embodiments, the starch content of the grain of the invention is between 25% and 65%, between 25% and 60%, between 25% and 55%, between 25% and 50%, between 30% and 70%, between 30% and 65%, between 30% and 60%, between 30% and 55%, or between 30% and 50%. In further embodiments, the starch content is about 35%, about 40%, about 45%, about 50%, about 55%, about 60% or about 65% as a percentage of the grain weight (w/w). The starch content of the grain of the invention may also be defined on a relative basis, i.e. relative to the starch content of corresponding wild-type grain. In embodiments, the starch content is between about 50% and about 90%, or between 50% and 80%, between 50% and 75%, between 50% and 70%, between 60% and 90% or between 60% and 80%, each being relative to that of wild-type grain. The starch content may also be between 90% and 100% relative to the starch content of wild-type grain. In each case, the comparison should be made by growing the plants under the same conditions, for example in field trials.

Flour, starch granules and bran of the invention may be obtained from grain by a milling process, optionally followed by a sifting or sieving process. Purified starch may also be obtained from grain by a milling process, for example a wet milling process, followed by further separation of the starch from protein, oil and fibre of the grain. As used herein, the term “milled product” refers to a product produced from grinding grain, preferably wheat grain of the invention, and includes flour (for example, wholemeal), middlings (also known as wheat midds), bran (including the germ) and starch granules. The middlings are usually in the form of granular particles and include bran and germ from the grain, and typically comprise about 18% protein, 20-30% starch, and about 5-6% lipid. This fraction from the milling process is relatively nutrient dense compared to white flour. Grain shape is a feature that can impact on the commercial usefulness of a plant, since grain shape can have an impact on the ease or otherwise with which the grain can be milled. The milling yield is an important parameter for commercial usefulness of the grain. The milling yield of the grain of the invention may be reduced by at least 10% relative to wild-type grain, but alternatively may be about the same as wild-type grain.

In another aspect, the invention provides starch granules or starch obtained from the grain of the plant of the invention. The starch of the granules has an increased proportion of amylose and a reduced proportion of amylopectin relative to wild-type wheat starch granules. The initial product of the milling process is a mixture or composition which includes starch granules, such as for example, white flour or wholemeal, and the invention therefore encompasses such granules. The starch granules from wild-type wheat comprise starch granule-bound proteins including GBSS, SSI, SBEIIa and SBEIIb amongst other proteins and therefore the presence of these proteins distinguish wheat starch granules from starch granules of other cereals. In contrast, the starch granules from wheat grain of the invention comprise wheat GBSS, including the GBSS polypeptides encoded by each of the A, B and D genomes of hexaploid wheat, but are reduced for SSIIa polypeptide, indeed some embodiments lack SSIIa polypeptide. These starch granules may also comprise reduced levels of one or more or all of the wheat SSI, SBEIIa and SBEIIb polypeptides, even if the wheat grain is wild-type for the genes encoding these enzymes. The starch granules from the wheat grain of the invention are typically distorted in shape and surface morphology when observed under light microscopy, see for Example 7, particularly for wheat grain having an amylose content of at least 45% or at least 50% as a percentage of the total starch of the grain. In an embodiment, at least 50%, preferably at least 60% or at least 70%, more preferably at least 80% of the starch granules obtained from the grain of the invention show distorted shape and/or surface morphology. The starch granules also show a loss of birefringence when observed under polarised light. See, for instance, Example 7 herein for determining the incidence of birefringence. For example, less than 50% or less than 25% of the starch granules show the “maltese cross” that is observed when wild-type starch granules are observed under polarised light.

The starch from starch granules may be purified by removal of the proteins after disruption and dispersal of the starch granules by heat and/or chemical treatment. The starch of the grain, the starch of the starch granules, and the purified starch of the invention may be further characterized by one or more or all of the following properties:
i) its amylose content is at least 45% (w/w), preferably between 45% and 70% on a weight basis, or at least 50% (w/w), or about 60% (w/w) amylose as a proportion of the total starch;
ii) comprising at least 2% resistant starch, preferably at least 3% resistant starch;
iii) the starch is characterised by a reduced glycaemic index (GI);
iv) the starch granules being distorted in shape;
v) the starch granules having reduced birefringence when observed under polarized light;
vi) the starch characterized by a reduced swelling volume;
vii) modified chain length distribution and/or branching frequency in the starch;
viii) the starch characterized by a reduced peak temperature of gelatinisation;
ix) the starch characterized by a reduced peak viscosity;
x) reduced starch pasting temperature;
xi) reduced peak molecular weight of amylose as determined by size exclusion chromatography;
xii) reduced starch crystallinity; and
xiii) reduced proportion of A-type and/or B-type starch, and/or increased proportion of V-type crystalline starch;
   each property being relative to wild-type wheat starch granules or starch.

The flour or starch of the invention may also be characterized by its swelling volume in heated excess water compared to wild-type flour or starch. Swelling volume is typically measured by mixing either a starch or flour with excess water and heating to elevated temperatures, typically greater than 90° C. The sample is then collected by centrifugation and the swelling volume is expressed as the mass of the sedimented material divided by the dry weight of the sample. A low swelling characteristic is useful where it is desired to increase the starch content of a food preparation, in particular a hydrated food preparation. The flour and starch of the invention preferably has a decreased swelling volume, such as for example decreased by 30-70% relative to a wild-type wheat flour or starch.

One measure of an altered amylopectin structure is the distribution of chain lengths, or the degree of polymerization, of the starch. The chain length distribution may be determined by using fluorophore-assisted carbohydrate electrophoresis (FACE) following isoamylase de-branching. The amylopectin of the starch of the invention may have a distribution of chain lengths in the range from 5 to 60, or in a subrange such as DP 7-11, that is greater than the corresponding chain length distribution of starch from wild-type plants, and/or reduced in frequency in other subranges, for example DP 12-24. See for instance, Example 7 herein. Starch with longer chain lengths will also have a commensurate decrease in frequency of branching. The starch of the grain of the invention is distinctively different to the starch of wheat grain having reduced SBEIIa activity (Regina et al., 2006) where the amylopectin of the grain comprises an increased proportion of the DP 4-12 chain length fraction relative to the amylopectin of wild-type grain, as measured after isoamylase debranching of the amylopectin. The difference can be readily determined by FACE.

In another aspect of the invention, the wheat starch may have an altered gelatinisation temperature, preferably a reduced gelatinisation temperature, which is readily measured by differential scanning calorimetry (DSC). Gelatinisation is the heat-driven collapse (disruption) of molecular order within the starch granule in excess water, with concomitant and irreversible changes in properties such as granular swelling, crystallite melting, loss of birefringence, viscosity development and starch solubilisation. The gelatinisation temperature may be either increased or decreased compared to starch from wild-type plants, depending on the chain length of the remaining amylopectin. High amylose starch from amylose extender (ae) mutants of maize showed a higher gelatinisation temperature than normal maize (Fuwa el al., 1999; Krueger et al., 1987).

The gelatinisation temperature, in particular the temperature of onset of the first peak or the temperature for the apex of the first peak, may be reduced by at least 3° C., preferably at least 5° C. or more preferably at least 7° C. as measured by DSC compared to starch extracted from a similar, but unaltered grain. The starch may comprise an elevated level of resistant starch, with an altered structure indicated by specific physical characteristics including one or more of the group consisting of physical inaccessibility to digestive enzymes which may be by reason of having altered starch granule morphology, the presence of appreciable starch associated lipid, altered crystallinity, and altered amylopectin chain length distribution. The high proportion of amylose also contributes to the level of resistant starch when the starch has been heated and then cooled.

The starch structure of the wheat of the present invention may also differ in that the degree of crystallinity is reduced and/or the type of crystallinity is modified compared to starch isolated from wild-type wheat grain. Crystallinity is typically investigated by X-ray crystallography. The reduced crystallinity of a starch is also thought to be associated with enhance organoleptic properties and contributes to a smoother mouth feel.

The invention also provides wheat grain, flour, wholemeal, starch granules and starch from the grain comprising increased amounts of dietary fibre, by way of at least an elevated level of RS, but also by way of increased levels of other dietary fibre components such as arabinoxylans, β-glucan and fructans. As used herein, “dietary fibre” (DF) or “total dietary fibre” (TDF) means the sum of carbohydrate polymers in food that are not digested in the small intestine of a healthy human subject and that have physiological benefit in the large intestine. As used herein, DF is different to “total fiber content” (below). DF is not digested and absorbed in the small intestine but passes to the colon where it may be degraded by bacteria. DF includes RS, non-α-glucan oligosaccharides and non-starch polysaccharides (NSP) such as arabinoxylans, β-glucan and fructans. DF is usually divided into forms which are soluble in water (soluble fibre) or not (insoluble fibre), and RS. The most-health promoting fraction of dietary fibre in wild-type wheat grain is the soluble fibre which mostly comprises the AX component, since wild-type starch is very low in RS. In contrast, in oats and barley the soluble fibre is mostly BG. The National Heart Foundation of Australia has recommended a daily intake of 30-35 g of DF for cardiovascular and colonic health. A variety of candidate genes have been identified in wheat which affect dietary fibre content (Quraishi et al., 2011), but none to the level as provided by the grain of the invention. DF may be measured by the method AOAC 991.43 (Megazyme).

As used herein, a “prebiotic” is a non-digestible (by human digestive enzymes) food ingredient that beneficially affects a subject by selectively stimulating the growth and/or activity of one or a limited number of bacteria in the colon after passing through the small intestine. For example, fructans and BG cannot be digested except through bacterial activity, but can alter the composition of human gut microbes by specific fermentation, producing short chain carboxylic acids including acetate, propionate and butyrate.

In embodiments, the wheat grain, flour, starch granules and starch of the invention provide modified digestive properties such as increased resistant starch. As used herein, “resistant starch” (RS) refers to the starch and products of starch digestion that are not absorbed in the small intestine of healthy individuals but enter into the large bowel. This is defined in terms of a percentage of the total starch of the grain, or a percentage of the total starch content in the food, according to the context. Therefore, resistant starch excludes products digested and absorbed in the small intestine. RS is therefore part of the dietary fibre content of the food ingredient (flour etc) or food product of the invention. RS is divided into five categories: physically inaccessible starch (RSI) such as, for example, in incompletely milled grain, resistant starch granules (RSII) such as, for example, found to a small extent in potatoes and green bananas, retrograded starch (RSIII) which is formed when gelatinised starch is cooled for an extended period of time, chemically modified starch (RSIV) such as formed by etherifying or esterifying free hydroxyl groups on the glycosyl residues, and starch capable of forming complexes between amylose or long branch chains of amylopectin with lipids (RSV) (Birt et al., 2013). RSIII is especially formed from longer chains of amylose which tend to recrystallize and form retrograded starch after gelatinisation. The RS in products based on starch with an elevated amylose content is mainly retrograded amylose (Hung et al., 2006). The increased RS of the starch of the grain, starch granules, starch and products therefrom of the invention is thought to be due to an increase in the RSII and RSV contents and to RSIII after retrogradation if the starch has been heated and then cooled, relative to the corresponding wild-type product. Starch-lipid association as measured by V-complex crystallinity is also likely to contribute to the level of resistant starch, increasing the RSV component by virtue of increased lipid content in the grain of the invention. Some of the starch may also be in an RSI form, being somewhat inaccessible to digestion.

Several methods are available to measure RS levels in food ingredients or food, all relying on an initial removal of digestible starch using starch hydrolysing enzymes (Dupuis et al., 2014). RS estimation by the Prosky method (AOAC 985.29) uses gravimetric determination of dietary fibre after α-amylase, glucoamylase and protease digestion (Prosky et al., 1985). The McCleary method (AOAC 2009.01) is the official method of the AOAC and is commercially available (Megazyme International, Ireland). It is the preferred method of measuring RS, see Example 1 herein. In this assay, non-resistant starch is solubilized by treatment with pancreatic α-amylase, the RS recovered and dissolved in 2 M KOH, and then hydrolysed to glucose with amyloglucosidase and measured.

In embodiments, the starch has between 2% and 20%, between 2% and 18%, between 3% and 18%, between 3% and 15%, or between 5% and 15% resistant starch on a weight basis, as a percentage of the total starch content. In embodiments, the RS content is increased by between 2- and 10-fold relative to a corresponding food ingredient or food product made with an equivalent amount of wild-type wheat starch. In embodiments, the RS content is increased by about 3-fold, about 4-fold, about 5-fold, about 6-fold, about 7-fold, about 8-fold, or about 9-fold relative to the wild-type. The extent of increased RS can be adjusted by blending the products of the invention with a corresponding wild-type product. The altered starch structure and in particular the high amylose levels of the starch of the invention give rise to an increase in RS when consumed in food. RS has beneficial physiological effects associated with metabolic products released during its fermentation in the bowel (Topping and Clifton, 2001), in particular the SCFA butyrate, propionate and acetate, and is effective in reducing postprandial blood glucose levels.

The grain, food ingredients and food products produced therefrom of the invention can be used advantageously for the provision in the diet of, or production of, compositions enriched for β-glucan, cellulose, fructan or arabinoxylan, based on the increased levels of these components in the grain of the invention. The cell walls of cereal grain are complex and dynamic structures composed of a variety of polysaccharides such as cellulose, xyloglucans, pectin (rich in galacturonic acid residues), callose (1,3-β-D-glucan), arabinoxylans (arabino-1,4-β-D-xylan, hereinafter AX) and BG, as well as polyphenolics such as lignin. In cell walls of the grasses and some other monocot plants, glucuronoarabinoxylans and BG predominate and the levels of pectic polysaccharides, glucomannans and xyloglucans are relatively low (Carpita et al., 1993). These polysaccharides are synthesized by a large number of diverse polysaccharide synthases and glycosyltranferases, with at least 70 gene families present in plants and in many cases, multiple members of gene families.

As used herein, the term “(1,3;1,4)-β-D-glucan”, also referred to as “β-glucan” and abbreviated herein as “BG”, refers to an essentially linear polymer of unsubstituted and essentially unbranched β-glucopyranosyl monomers covalently linked mostly through 1,4-linkages with some 1,3-linkages. The glucopyranosyl residues, joined by 1,4- and 1,3-linkages, are arranged in a non-repeating but non-random fashion, i.e. the 1,4- and 1,3-linkages are not arranged randomly, but equally they are not arranged in regular, repeating sequences (Fincher, 2009a, 2009b). Most (about 90%) of the 1,3-linked residues follow 2 or 3 1,4-linked residues in wheat BG, as in oat and barley BG. BG can therefore be considered to be a chain of mainly β-1,4 linked cellotriosyl (each with 3 glucopyranosyl residues) and cellotetrosyl (each with 4 glucopyranosyl residues) units linked together by single β-1,3 linkages with approximately 10% longer β-1.4 linked cellodextrin units of four to about ten 1,4-linked glucopyranosyl residues, up to about 28 glucopyranosyl residues (Fincher and Stone, 2004). Typically, the BG polymers have at least 1000 glycosyl residues and adopt an extended conformation in aqueous media. The ratio of tri- to tetra-saccharide units (DP3/DP4 ratio) varies among species and therefore is characteristic of BG from a species. BG from different cereals differ in their solubility, with BG from oats being more soluble than the BG from wheat. This is thought to be related to the DP3 to DP4 ratio of the BG polymer.

In wild-type wheat grain, BG levels are greater in the whole grain than in the endosperm (Henry, 1985). BG content of wild-type whole wheat grain was about 0.6% on a weight basis, compared to about 4.2% for barley, 3.9% for oats and 2.5% for rye (Henry 1987). In wild-type wheat grain, the range was 0.4-1.4% by weight (Lazaridou et al., 2007). Wheat grain BG typically has a DP3/DP4 ratio of 3-4.5 (Lazaridou et al., 2007). Whilst barley BG has been associated with lowering plasma cholesterol, reducing glycemic index and reducing the risk of colon cancer, wheat BG has not been associated with these effects since wheat grain has much lower BG levels than barley. The level of BG in grain is ordinarily measured by milling the grain to wholemeal and assaying for BG by, for example, the method described in Example 1.

In wild-type wheat grain, the level of fructan is only 0.6%-2.6% by weight of the grain. As used herein, the term “fructan” means polymers of fructose which comprise fructosyl residues polymerized to a single terminal glucose unit. Fructans are synthesized from sucrose, explaining the terminal glucose. The fructose moieties are linked to each other by β-1,2 and/or β-2,6 bonds, and the glucose may be linked to the end of the chain by an α-1,2 bond as occurs in sucrose, being formed by repeated fructosyl transfer from sucrose. The enzymes involved in fructan synthesis include sucrose-sucrose fructosyl transferase (EC 2.4.1.99) which forms ketose, and either 1- or 6-fructan-fructan fructosyl transferase (EC 2.4.1.100). The degree of polymerisation (DP) varies from 3 to several hundred but is typically 3-60 and in grain of the invention mostly DP 3-10. In view of this composition, fructans are highly soluble in water and do not precipitate in 78% ethanol. The linkages between the fructosyl-residues are either exclusively of the β-1,2 type forming a linear molecule (inulin) in which the fructosyl residues are attached to the fructosyl residue of the sucrose starter, or of the β-2,6 type (levan), or both linkage types occur in branched fructans (graminans). Graminans, which comprise β-2,6-linked fructose units with β-1,2 branch points and are therefore more complex structures, can also be present in cereals, and can be mixed with levans. The level of fructan in grain of the invention is ordinarily measured by milling the grain to wholemeal and assaying for fructan by, for example, the method described in Example 1, which is based on that of Prosky and Hoebregs (1999). The method depends on the hydrolysis of the fructans followed by determination of the released sugars.

Fructans are non-starch carbohydrates with potentially beneficial effects as a food ingredient on human health (Tungland and Meyer; 2002; Ritsema and Smeekens, 2003). The human digestive enzymes α-glucosidase, maltase, isomaltase and sucrase are not able to hydrolyse fructans because of the β-configuration of the fructan linkages. Furthermore, humans and other mammals lack, in their small intestines, the fructan exohydrolase enzymes that break down fructans and therefore dietary fructans avoid digestion in the small intestine and reach the large intestine intact. However, bacteria there are able to ferment fructans and thereby utilize them as, for example, an energy or carbon source for growth and production of short-chain fatty acids (SCFA). Dietary fructans therefore are able to stimulate the growth of beneficial bacteria such as bifidobacteria in the colon, which aids in prevention of bowel disorders such as constipation and infection by pathogenic gut bacteria. Dietary fructan also enhances nutrient absorption from diets, particularly calcium and iron, possibly via production of SCFA which in turn reduce luminal pH and modify calcium speciation and hence solubility, or exert a direct effect on the mucosal transport pathway, thereby improving the mineralization of bone and reducing the risk of iron deficiency anaemia. In addition, a high-fructan diet can improve the health of patients with diabetes and reduce the risk of colonic cancers by suppressing aberrant crypt foci which are precursors of colon cancer (Kaur and Gupta, 2002). Also, fructans have a sweet taste and are increasingly used as low-calorie sweeteners and as functional food ingredients.

Production of isolated fructan from grain of the invention is cost-effective relative to existing methods of fructan production, for example, involving the extraction of inulins from chicory. Large scale extraction of fructan can be achieved by milling the grain to wholemeal flour and then extracting the total sugars including fructans from the flour into water. This may be done at ambient temperature and the mixture then centrifuged or filtered. The supernatant is then heated to about 80° C. and centrifuged to remove proteins, then dried down. Alternatively, the extraction of flour can be done using 80% ethanol, with subsequent phase separation using water/chloroform mixtures, and the aqueous phase containing sugars and fructan dried and redissolved in water. Sucrose in the extract prepared either way may be removed enzymatically by the addition of α-glucosidase, and then hexoses (monosaccharides) removed by gel filtration to produce fructan fractions of various sizes. This would produce a fructan enriched fraction of at least 50%, preferably at least 60% or at least 70%, more preferably at least 80% fructan.

Development of a Small-Scale Fructan Assay.

The fructan content in cereal grains and their derived food products is commonly measured using high-performance liquid chromatography (HPLC) (Huynh et al., 2008) or spectrophotometry (McCleary et al., 2000; McCleary et al., 2013; Steegmans et al., 2004). The Official AOAC Method 999.03 (AOAC, 2000b) based on spectrophotometry has been commercialized as the K-FRUCHK and K-FRUC kits by Megazyme International Limited (Bray, Ireland). These commercial kits are convenient and easy for measuring fructan levels in cereal grains (Karppinen et al., 2003; Whelan et al., 2011). In the K-FRUCHK assay, sucrose and low degree-of polymerisation (DP) maltosaccharides are hydrolysed to fructose and glucose. Their concentration is measured with a hexokinase/phosphor-glucose isomerise/glucose 6-phosphate dehydrogenase (HK/PGI/G6PGH) system using a spectrophotometer. After fructan hydrolysis, the total concentration of fructose and glucose is re-measured and the fructan content is then determined by the difference between the two measurements. In the K-FRUC assay, sucrose, maltose, maltodextrins and starch are hydrolysed to fructose and glucose that are further reduced by sodium borohydride to the corresponding sugar alcohols (sorbitol and mannitol). Fructose and glucose derived from fructan hydrolysis are coupled with 4-hydroxybenzoic acid hydrazide (PAHBAH) to develop colour in a boiling water bath and their absorbance is read using a spectrophotometer for calculating fructan content.

A simplified enzymatic hydrolysis followed by the HPLC analysis was recently developed for screening fructan content in a double haploid (DH) wheat population (Huynh et al., 2008b). There is a need for accurate and rapid measurement of fructan content in large breeding populations with hundreds to thousands of lines. However, all the current fructan assays used in cereal grains are relatively low efficiency, allowing about 10 samples per day per worker, and require several grams of each flour sample. In order to develop high throughput fructan assays, the inventors scaled down the K-FRUCHK and K-FRUC assays in a plate format to allow this, as described in Example 1.

Arabinoxylan is another polysaccharide found in plant cell walls, including in the cell walls in wheat grain. The levels of arabinoxylan in the grain and flour of the invention are increased relative to the corresponding wild-type grain and flour, for example by at least 1.5-fold or at least 2-fold on a weight basis. The level may be increased between 1.5-fold and 3-fold. As used herein, “arabinoxylan” (AX) refers to a linear chain backbone of β-D-xylopyranosyl residues linked through 1,4 glycosidic linkages, with α-L-arabinofuranosyl residues attached to some of the xylopyranosyl residues at O-2, 0-3 and/or at both O-2,3 positions. The xylopyranosyl residues are any one of: mono-substituted at O-2 or O-3, di-substituted at O-2,3, and unsubstituted. Sidebranches may contain, in addition to arabinose residues, small amounts of xylopyranose, galactopyranose, α-D-glucuronic acid or 4-O-methyl-α-D-glucuronic residues. In cereals, the Ara/Xyl ratio may vary from 0.3 to 1.1. AX from the outer pericarp, scutellum and embryonic axis are relatively highly substituted with arabinose whereas those of the aleurone and hyaline layer are less substituted. AX may further comprise the hydrocinnamic acids, ferulic acid and p-coumaric acid, esterified to O-5 of arabinose residues linked to the O-3 of the xylose residues (Smith and Hartley, 1983). The biosynthesis of the 1,4-β-D-xylan backbone is catalyzed by 1,4-β-xylosyl transferase that uses UDP-D-xylose as substrate and transfers the xylose unit to the non-reducing end of an xylooligosaccharide chain.

The synthesis of AX in cereals involves xylotransferases that use UDP-Xyl as substrate and may involve a complex tetrasaccharide as a primer (Carpita et al., 2011). Arabinoxylans are only slightly soluble in water and require alkali solvents for their efficient extraction. For example, barium hydroxide can selectively extract AX (Gruppen et al., 1992). In contrast, sodium hydroxide extracts both BG and AX. The level of AX in grain is ordinarily measured by milling the grain to wholemeal and assaying for AX by, for example, the method described in Example 1.

Studies using maize, rye and wheat AX demonstrated positive effects on cecal fermentation, production of SCFA, reduction in serum cholesterol and improved absorption of calcium and magnesium (Hopkins et al. 2003).

As used herein, cellulose refers to a crystalline array of about 24-36 (1-4)-β-D-glucan chains that form microfibrils, found predominantly in the cell walls of plants. It is one of the most abundant polymers found in nature. The glucan chains are formed by cellulose synthases of the CesA gene family at the plasma membrane (Giddings et al., 1980), and 24 to 36 chains are then assembled into a functional microfibril. Arabidopsis has 10 CesA genes, at least 3 of which are co-expressed during primary cell wall formation and three others during secondary cell wall formation (Carpita et al., 2011), each of which add glycosyl residues to the non-reducing end of acceptor glucan chains to extend the polymers. The CesA genes are related to the Csl genes of both monocots and dicots which are involved in synthesis of other polysaccharides. For example, the CslF and CslH enzymes found only in the grasses including cereals are involved in synthesis of BG.

The food products made from the grain, food ingredients, starch granules and starch are characterized by a decreased Glycaemic Index. GI is a simple marker for the effect of carbohydrate rich foods on post-prandial glucose levels in the blood of human subjects. As used herein, “Glycaemic Index” or “GI” means a measure of the area under the curve of blood glucose concentrations after eating a portion of a test food containing 50 g of carbohydrate, divided by the incremental area achieved with the same amount of carbohydrate present in an equivalent amount of glucose or white bread. GI therefore relates to the rate of digestion of foods comprising the starch and uptake of the digestion products, and is a comparison of the effect of a test food with the effect of white bread or glucose on excursions in blood glucose concentration. GI is thereby a measure of the effect of the food on post prandial serum glucose concentration and is associated with the demand for insulin for blood glucose homeostasis. One important characteristic provided by foods of the invention is a reduced GI relative to a corresponding food made with the same amount of wild-type wheat, flour, starch granules or starch as food ingredient. Furthermore, the foods of the invention may have a reduced level of final digestion and consequently be relatively low-calorie compared to a corresponding food made with the same amount of wild-type wheat as food ingredient. A low calorific product might be based on inclusion of flour produced from milled wheat grain. Such foods may have the effect of being filling, enhancing bowel health, reducing the post-prandial serum glucose and lipid concentration as well as providing for a low calorific food product.

GI of starch of the invention, or a food ingredient or food product of the invention, is readily measured using an in vitro assay as described in Example 9 herein. The in vitro assay simulates the digestion of the starch in the products as occurs upon consumption in healthy humans, and is predictive of the GI as measured in human subjects after consumption of the products.

The method of treating the subject, particularly humans, may comprise the step of administering altered wheat grain, flour, starch or a food or drink product as defined herein to the subject, in one or more doses, in an amount and for a period of time whereby the level of the one or more of bowel health or metabolic indicators improves. The indicator may change relative to consumption of a corresponding non-altered wheat starch or wheat or product thereof, within a time period of up to 24 hours, as in the case of some of the indicators such as pH, elevation of levels of SCFA, post-prandial glucose fluctuation, or it may take days such as in the case of increase in fecal bulk or improved laxation, or perhaps longer in the order of weeks or months such as in the case where the butyrate enhanced proliferation of normal colonocytes is measured. It may be desirable that administration of the altered starch or wheat or wheat product be lifelong. However, there are good prospects for compliance by the individual being treated given the relative ease with which the altered starch can be administered.

Dosages may vary depending on the condition being treated or prevented but are envisaged for humans as being at least 10 g of wheat grain or starch of the invention per day, more preferably at least 15 g per day, preferably at least 20 or at least 30 g per day. Administration of greater than about 100 grams per day may require considerable volumes of delivery and reduce compliance. Most preferably the dosage for a human is between 10 and 100 g of wheat grain of the invention, or flour, wholemeal or modified starch of the invention per day, or for adult humans between 20 and 100 g per day.

The indicators of improved bowel health may comprise, but are not necessarily limited to:
i) decreased pH of the bowel contents,
ii) increased total SCFA concentration or total SCFA amount in the bowel contents,
iii) increased concentration or amount of one or more SCFAs in the bowel contents,
iv) increased fecal bulk,
v) increase in total water volume of bowel or faeces, without diarrhea,
vi) improved laxation,
vii) increase in number or activity of one or more species of probiotic bacteria,
viii) increase in fecal bile acid excretion,
ix) reduced urinary levels of putrefactive products,
x) reduced fecal levels of putrefactive products,
xi) increased proliferation of normal colonocytes,
xii) reduced inflammation in the bowel of individuals with inflamed bowel or a tendency to inflamed bowel,
xiii) reduced fecal or large bowel levels of any one of urea, creatinine and phosphate in uremic patients, and
xiv) any combination of the above.

The indicators of improved metabolic health may comprise, but are not necessarily limited to:
i) stabilisation of post-prandial glucose fluctuation,
ii) improved (lowered) glycaemic response,
iii) reduced pro-prandial plasma insulin concentration,
iv) improved blood lipid profile,
v) lowering of plasma LDL cholesterol,
vi) reduced plasma levels of one or more of urea, creatinine and phosphate in uremic patients,
vii) an improvement in a dysglucaemic response, or
viii) any combination of the above.

The pH of the bowel contents may be decreased by at least 0.1 units, preferably by at least 0.15 or 0.2 units. Each of the other indicators of bowel health or metabolic health may be improved by at least 10%, preferably at least 20%.

It will be understood that one benefit of the present invention is that it provides for products such as bread that are of particular nutritional benefit, and moreover it does so without the need to post-harvest modify the starch or other constituents of the wheat grain. However, it may be desired to make modifications to the starch or other constituent of the grain, and the invention encompasses such a modified constituent. Methods of modification are well known and include the extraction of the starch or other constituent by conventional methods and modification of the starches to increase the resistant form. The starch may be modified by treatment with heat and/or moisture, physically (for example ball milling), enzymatically (using for example α- or β-amylase, pullalanase or the like), chemical hydrolysis (wet or dry using liquid or gaseous reagents), oxidation, cross bonding with difunctional reagents (for example sodium trimetaphosphate, phosphorus oxychloride), or carboxymethylation.

Whilst the invention may be particularly useful in the treatment or prophylaxis of humans, it is to be understood that the invention is also applicable to non-human subjects including but not limited to agricultural animals such as cows, sheep, pigs and the like, domestic animals such as dogs or cats, laboratory animals such as rabbits or rodents such as mice, rats, hamsters, or animals that might be used for sport such as horses. The method may be particularly applicable to non-ruminant mammals or animals such as mono-gastric mammals. The invention may also be applicable to other agricultural animals for example poultry including, for example, chicken, geese, ducks, turkeys, or quails, or fish.

The terms “polypeptide” and “protein” are generally used interchangeably herein. The terms “proteins” and “polypeptides” as used herein also include variants, mutants, modifications and/or derivatives of the polypeptides of the invention as described herein. As used herein, “substantially purified polypeptide” refers to a polypeptide that has been separated from the lipids, nucleic acids, other peptides and other molecules with which it is associated in its native state. Preferably, the substantially purified polypeptide is at least 60% free, more preferably at least 75% free, and more preferably at least 90% free from other components with which it is naturally associated. By “recombinant polypeptide” is meant a polypeptide made using recombinant techniques, i.e., through the expression of a recombinant polynucleotide in a cell, preferably a plant cell and more preferably a wheat cell. In an embodiment, the polypeptide has starch synthase enzyme activity, particularly SSIIa activity, and is at least 98% identical to a SSIIa polypeptide described herein.

The % identity of a polypeptide relative to a reference polypeptide can be determined by any program known in the art for aligning amino acid sequences, such as the program GAP (Needleman and Wunsch, 1970, GCG program) with a gap creation penalty=5, and a gap extension penalty=0.3. The analysis aligns the two sequences over the full length amino acid sequence of the reference sequence. For example, if the reference sequence is the amino acid sequence set forth as SEQ ID NO:1, the alignment is along the full length of SEQ ID NO:1. A gap in an aligned sequence is regarded as a position of non-identity for each missing amino acid.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence deletions or insertions generally range from about 1 to 15 residues, more preferably about 1 to 10 residues and typically about 1 to 5 contiguous residues. However, they may be larger than 15 amino acids, up to the full length of the polypeptide. The polypeptide sequence may be a truncated sequence relative to the corresponding wild-type sequence or reference SEQ ID NO. For example, if the protein coding region encoding the polypeptide has a premature translation termination codon (stop codon), the resultant polypeptide will, if translated, be truncated. The extent of the truncation depends on the position of the stop codon, shortening the polypeptide by at least 5%, preferably at least 10% relative to the wild-type sequence.

The protein coding region of a gene of the invention may be disrupted by the presence of a splice-site mutation which causes mis-splicing and may result in an altered RNA transcript such that the open reading frame is disrupted. Then the amino acid sequence of the polypeptide may be identical to the wild-type up to the point of the mis-splicing or downstream of that point and then diverge from the wild-type. Such polypeptides are generally affected in their activity in the same way as a truncated polypeptide. Examples of stop codons and splice-site mutations in an SSIIa gene are described in Example 11 herein.

Substitution mutants have at least one amino acid residue in the polypeptide removed and a different residue inserted in its place. The sites of greatest interest for substitutional mutagenesis for reduced activity of the polypeptide include sites identified as the active site(s). Other sites of interest are those in which particular residues obtained from various strains or species are identical i.e. conserved amino acids. These positions are likely to be important for biological activity. These amino acids, especially those falling within a contiguous sequence of at least three other identically conserved amino acids, are preferably substituted in a relatively conservative manner in order to retain function such as SSIIa enzyme activity, or in a non-conservative manner for reduced activity. Conservative substitutions are shown in Table 3 under the heading of “exemplary substitutions”. “Non-conservative amino acid substitutions” are defined herein as substitutions other than those listed in Table 3 (Exemplary conservative substitutions). Non-conservative substitutions in an SSIIa polypeptide are expected to reduce the activity of the enzyme and many will correspond to an SSIIa encoded by a “partial loss of function mutant SSIIa gene”.

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

TABLE 3
Exemplary and Preferred Conserved Amino Acid Substitutions

| | Exemplary conservative | Preferred conservative |
|---|---|---|
| Original Residue | substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe | Leu |
| Leu | Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

In some embodiments, the present invention involves modification of gene activity, particularly of SSIIa gene activity, combinations of mutant genes, and the construction and use of chimeric genes. As used herein, the term “gene” includes any deoxyribonucleotide sequence which includes a protein coding region or which is transcribed in a cell but not translated, together with associated non-coding and regulatory regions. The term “gene” also includes mutant forms of a wild-type gene, which mutant genes may not be transcribed and/or translated, such as, for example, if a promoter region has been deleted. Associated non-coding and regulatory regions are typically located adjacent to the protein coding region on both the 5′ and 3′ ends for a distance of about 2 kb on either side. In this regard, the gene includes control signals such as promoters, enhancers, transcription termination and/or polyadenylation signals that are naturally associated with a given gene, or heterologous control signals in which case the gene is referred to as a “chimeric gene”. The sequences which are located 5′ of the protein coding region and which are present on the mRNA are referred to as 5′ non-translated sequences (5′-UTR). The sequences which are located 3′ or downstream of the protein coding region and which are present on the mRNA are referred to as 3′ non-translated sequences (3′-UTR). The term “gene” encompasses both cDNA and genomic forms of a gene. A “cDNA” is a DNA copy of an RNA transcript of a gene and is described herein as “corresponding to the gene”. For example, the cDNA nucleotide sequence set forth as SEQ ID NO:4 corresponds to the SSIIa-A gene whose wild-type sequence is set forth as SEQ ID NO:7. The term “gene” includes synthetic or fusion molecules encoding the proteins of the invention described herein. Genes are ordinarily present in the wheat genome as double-stranded DNA. A chimeric gene may be introduced into an appropriate vector for extrachromosomal maintenance in a cell or for integration into the host genome. As used herein, genes or genotypes are referred to in italicised form (e.g. SSIIa) while proteins, enzymes or phenotypes are referred to in non-italicised form (SSIIa).

As used herein, the term “genotype” refers to the genetic makeup of a wheat cell, tissue, plant, plant part or plant product. The genetic makeup will be identical to that of the wheat plant from which the product was obtained. As used herein, the term “phenotype” refers to an observable characteristic, or set of multiple characteristics, of the cell, tissue, plant, plant part or plant product which result from the interaction between the plant's genotype and the environment under which the plant was grown.

A genomic form or clone of a gene containing the coding region may be interrupted with non-coding sequences termed “introns” or “intervening regions” or “intervening sequences.” An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in an encoded polypeptide.

The present invention refers to various polynucleotides. As used herein, a “polynucleotide” or “nucleic acid” or “nucleic acid molecule” means a polymer of nucleotides, which may be DNA or RNA and includes for example cDNA, mRNA, tRNA, siRNA, shRNA, hpRNA, and single or double-stranded DNA. It may be DNA or RNA of cellular, genomic or synthetic origin. Preferably the polynucleotide is solely DNA or solely RNA as occurs in a cell. The polymer may be single-stranded, essentially double-stranded or partly double-stranded. An example of a partly-double stranded RNA molecule is a hairpin RNA (hpRNA), short hairpin RNA (shRNA) or self-complementary RNA which includes a double stranded stem formed by basepairing between a nucleotide sequence and its complement and a loop sequence which covalently joins the nucleotide sequence and its complement. Basepairing as used herein refers to standard basepairing between nucleotides, including G:U basepairs in an RNA molecule. “Complementary” means two polynucleotides are capable of basepairing along part of their lengths, or along the full length of one or both (fully complementary).

By “isolated” is meant material that is substantially or essentially free from components that normally accompany it in its native state. As used herein, an “isolated polynucleotide” or “isolated nucleic acid molecule” means a polynucleotide which is at least partially separated from, preferably substantially or essentially free of, the polynucleotide sequences of the same type with which it is associated or linked in its native state. For example, an “isolated polynucleotide” includes a polynucleotide which has been purified or separated from the sequences which flank it in a naturally occurring state, e.g., a. DNA fragment which has been removed from the sequences which are normally adjacent to the fragment. Preferably, the isolated polynucleotide is also at least 90% free from other components such as proteins, carbohydrates, lipids etc. The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably connected to the nucleotide sequence to be transcribed in the cell.

The present invention refers to use of oligonucleotides which may be used as “probes” or “primers”. As used herein, “oligonucleotides” are polynucleotides up to 50 nucleotides in length, preferably 15-50 nucleotides in length. They can be RNA, DNA, or combinations or derivatives of either. Oligonucleotides are typically relatively short single stranded molecules of 10 to 30 nucleotides, commonly 15-25 nucleotides in length, typically comprised of 10-30 or 15-25 nucleotides which are identical to, or complementary to, part of an SSIIa gene or cDNA corresponding to an SSIIa gene. When used as a probe or as a primer in an amplification reaction, the minimum size of such an oligonucleotide is the size required for the formation of a stable hybrid between the oligonucleotide and a complementary sequence on a target nucleic acid molecule. Polynucleotides used as a probe are typically conjugated with a detectable label such as a radioisotope, an enzyme, biotin, a fluorescent molecule or a chemiluminescent molecule. Oligonucleotides and probes of the invention are useful in methods of detecting an allele of a SSIIa or other gene associated with a trait of interest, for example modified starch. Such methods employ nucleic acid hybridization and in many instances include oligonucleotide primer extension by a suitable polymerase, for example as used in PCR for detection or identification of wild-type or mutant alleles. Preferred oligonucleotides and probes hybridise to a SSIIa gene sequence from wheat or other cereals, including any of the sequences disclosed herein, for example SEQ ID NOs: 15 to 49. Preferred oligonucleotide pairs are those that span one or more introns, or a part of an intron and therefore may be used to amplify an intron sequence in a PCR reaction. Numerous examples are provided in the Examples herein.

The terms “polynucleotide variant” and “variant” and the like refer to polynucleotides displaying substantial sequence identity with a reference polynucleotide sequence and which are able to function in an analogous manner to, or with the same activity as, the reference sequence. These terms also encompass polynucleotides that are distinguished from a reference polynucleotide by the addition, deletion or substitution of at least one nucleotide, or that have, when compared to naturally occurring molecules, one or more mutations. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Accordingly, these terms encompass polynucleotides that encode polypeptides that exhibit enzymatic or other regulatory activity, or polynucleotides capable of serving as selective probes or other hybridising agents. The terms “polynucleotide variant” and “variant” also include naturally occurring allelic variants. Mutants can be either naturally occurring (that is to say, isolated from a natural source) or synthetic (for example, by performing site-directed mutagenesis on the nucleic acid). Preferably, a polynucleotide variant of the invention which encodes a polypeptide with enzyme activity is at least 90% in length relative to the wild-type, up to the full length of the gene.

A variant of an oligonucleotide of the invention includes molecules of varying sizes which are capable of hybridising, for example, to the wheat genome at a position close to that of the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close (for example, but not limited to, within 50 nucleotides) to the region of the plant genome where the specific oligonucleotides defined herein hybridise.

By “corresponds to” or “corresponding to” in the context of polynucleotides or polypeptides is meant a polynucleotide (a) having a nucleotide sequence that is substantially identical or complementary to at least a portion of, preferably all of (fully complementary), a reference polynucleotide sequence or (b) encoding an amino acid sequence identical to an amino acid sequence in a polypeptide. This phrase also includes within its scope a polypeptide having an amino acid sequence that is substantially identical to a sequence of amino acids in a reference polypeptide. Terms used to describe sequence relationships between two or more polynucleotides or polypeptides include “reference sequence”, “sequence identity”, “percentage of sequence identity”, “substantial identity” and “identical”, and are defined with respect to a defined minimum number of nucleotides or amino acid residues or preferably over the full length. The terms “sequence identity” and “identity” are used interchangeably herein to refer to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U) or the identical amino acid residue occurs in both sequences to yield the number of matched positions, dividing the number of matched positions by the total number of positions in the window of comparison (i.e., the window size), and multiplying the result by 100 to yield the percentage of sequence identity.

The % identity of a polynucleotide to a reference polynucleotide can be determined by any program known in the art for this purpose, such as for example, GAP (Needleman and Wunsch, 1970, GCG program) with a gap creation penalty=5, and a gap extension penalty=0.3. Reference also may be made to the BLAST family of programs as for example disclosed by Altschul et al., 1997. A detailed discussion of sequence analysis can be found in Unit 19.3 of Ausubel et al., 1994-1998, Chapter 15. Unless stated otherwise, the alignment is carried out along the full length of the reference sequence.

Nucleotide or amino acid sequences are indicated as “essentially similar” when such sequences have a sequence identity of at least 98%, more particularly at least about 98.5%, quite particularly about 99%, especially about 99.5%, more especially about 99.8%, and includes when the sequences are identical. It is clear that when RNA sequences are described as essentially similar to, or have a certain degree of sequence identity with, DNA sequences, thymine (T) in the DNA sequence is considered equal to uracil (U) in the RNA sequence.

With regard to the defined polynucleotides, it will be appreciated that higher % identity figures will encompass preferred embodiments. Thus, where applicable, in light of the minimum % identity figures, it is preferred that the polynucleotide comprises a polynucleotide sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In some embodiments, the present invention refers to the stringency of hybridization conditions to define the extent of complementarity of two polynucleotides. “Stringency” as used herein, refers to the temperature and ionic strength conditions, and presence or absence of certain organic solvents, during hybridization. The higher the stringency, the higher will be the degree of complementarity between a target nucleotide sequence and the labelled polynucleotide sequence. “Stringent conditions” refers to temperature and ionic conditions under which only nucleotide sequences having a high frequency of complementary bases will hybridize. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, herein incorporated by reference. Specific hybridization conditions referred to herein are as follows: 1) low stringency hybridization conditions in 6× sodium chloride/sodium citrate (SSC) at about 45° C., followed by two washes in 0.2×SSC, 0.1% SDS at 50-55° C.; 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5 M sodium phosphate, 7% SDS at 65° C., followed by one or more washes at 0.2×SSC, 1% SDS at 65° C.

As used herein, a “chimeric gene” or “genetic construct” refers to any gene that is not a native gene in its native location i.e. it has been artificially manipulated, including a chimeric gene or genetic construct which is integrated into the wheat genome. Typically a chimeric gene or genetic construct comprises regulatory and transcribed or protein coding sequences that are not found together in nature. Accordingly, a chimeric gene or genetic construct may comprise regulatory sequences and coding sequences that are derived from different sources, or regulatory sequences and coding sequences derived from the same source, but arranged in a manner different than that found in nature. The term “endogenous” is used herein to refer to a substance that is normally produced in an unmodified plant at the same developmental stage as the plant under investigation, preferably a wheat plant, such as starch or an SSIIa gene of SSIIa polypeptide. An “endogenous gene” refers to a native gene in its natural location in the genome of an organism, preferably an SSIIa gene in a wheat plant. The terms “foreign polynucleotide” or “exogenous polynucleotide” or “heterologous polynucleotide” and the like refer to any nucleic acid which is introduced into the genome of a cell by experimental manipulations, preferably the wheat genome, but which does not naturally occur in the cell. These include modified forms of gene sequences found in that cell so long as the introduced gene contains some modification, e.g. an introduced mutation or the presence of a selectable marker gene, relative to the naturally-occurring gene. Foreign or exogenous genes may be genes found in nature that are inserted into a non-native organism, native genes introduced into a new location within the native host, or chimeric genes or genetic constructs. The term “genetically modified” includes introducing genes into cells, mutating genes in cells and artificially altering or modulating the regulation of a gene in a cell by modifying the genome, or organisms to which these acts have been done or their progeny or parts such as grain.

The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides which regulates the expression of the genetic sequence. This may be a naturally occurring cis-acting sequence in its native context, for example regulating a wheat SSIIa gene, or a sequence in a genetic construct which when positioned appropriately relative to an expressible genetic sequence, regulates its expression. Such a cis-regulatory region may be capable of activating, silencing, enhancing, repressing or otherwise altering the level of expression and/or cell-type-specificity and/or developmental specificity of a gene sequence at the transcriptional or post-transcriptional level. For example, the presence of an intron in the 5′-leader (UTR) of genes has been shown to enhance expression of genes in monocotyledonous plants such as wheat (Tanaka et al., 1990). Another type of cis-acting sequence is a matrix attachment region (MAR) which may influence gene expression by anchoring active chromatin domains to the nuclear matrix.

By “vector” is meant a nucleic acid molecule, preferably a DNA molecule derived from a plasmid or plant virus, into which a nucleic acid sequence may be inserted. The vector may also include a selection marker such as an antibiotic resistance gene that can be used for selection of suitable bacterial or plant transformants, or sequences that enhance transformation of prokaryotic or eukaryotic (especially wheat) cells such as T-DNA or P-DNA sequences. Examples of such resistance genes and sequences are well known to those of skill in the art.

By “marker gene” is meant a gene that imparts a distinct phenotype to cells expressing the marker gene and thus allows such transformed cells to be distinguished from cells that do not have the marker, and are well known in the art. A “selectable marker gene” confers a trait for which one can ‘select’ based on resistance to a selective agent (e.g., a herbicide, antibiotic, radiation, heat, or other treatment damaging to untransformed cells) or based on a growth advantage in the presence of a metabolizable substrate. Exemplary selectable marker genes for selection of plant transformants include, but are not limited to, a hyg gene which confers hygromycin B resistance; a neomycin phosphotransferase (npt) gene conferring resistance to kanamycin and the like as, for example, described by Potrykus et al., 1985; a glutathione-S-transferase gene from rat liver conferring resistance to glutathione derived herbicides as, for example, described in EP-A-256223; a glutamine synthetase gene conferring, upon overexpression, resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A-275957, a gene encoding a 5-enolshikimate-3-phosphate synthase (EPSPS) conferring tolerance to N-phosphonomethylglycine as, for example, described by Hinchee et al., 1988, a bar gene conferring resistance against bialaphos as, for example, described in WO91/02071; or a nitrilase gene such as bxn from Klebsiella ozaenae which confers resistance to bromoxynil (Stalker et al., 1988). Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (GFP, Niedz et al., 1995) or one of its variants; a luciferase (luc) gene (Ow et al., 1986), which allows for bioluminescence detection, and others known in the art.

In some embodiments, the level of an enzyme activity is modulated by decreasing the level of expression of genes encoding the enzyme in the wheat plant, or increasing the level of expression of a nucleotide sequence that codes for the enzyme in a wheat plant. Increasing expression can be achieved at the level of transcription by using promoters of different strengths or inducible promoters, which are capable of controlling the level of transcript expressed from the coding sequence. Heterologous sequences may be introduced which encode transcription factors that modulate or enhance expression of genes whose products down-regulate starch synthesis such as SSIIa. The level of expression of the gene may be modulated by altering the copy number per cell of a construct comprising the coding sequence and a transcriptional control element that is operably connected thereto and that is functional in the cell. Alternatively, a plurality of transformants may be selected, and screened for those with a favourable level and/or specificity of transgene expression arising from influences of endogenous sequences in the vicinity of the transgene integration site. A favourable level and pattern of transgene expression is one which results in a substantial increase in amylose content in the wheat plant. This may be detected simply by testing the transformants.

Reducing gene expression may also be achieved through introduction and transcription of a “gene-silencing chimeric gene” introduced into the wheat plant. The gene-silencing chimeric gene is preferably introduced stably into the wheat genome, preferably the wheat nuclear genome, so that it is stably inherited in progeny. As used herein “gene-silencing effect” refers to the reduction of expression of a target nucleic acid in a wheat cell, preferably an endosperm cell during seed development while the plant is growing, which can be achieved by introduction of a silencing RNA. In a preferred embodiment, a gene-silencing chimeric gene is introduced which encodes an RNA molecule which reduces expression of one or more endogenous genes, for example genes other than the SSIIa genes, or preferably the three endogenous SSIIa genes. Such reduction may be the result of reduction of transcription, including by methylation of promoter regions via chromatin remodelling, or post-transcriptional modification of the RNA molecules, including via RNA degradation, or both. Gene-silencing should not necessarily be interpreted as an abolishing of the expression of the target nucleic acid or gene. It is sufficient that the level expression of the target nucleic acid in the presence of the silencing RNA is lower that in the absence thereof. The level of expression of the targeted gene may be reduced by at least about 40% or at least about 45% or at least about 50% or at least about 55% or at least about 60% or at least about 65% or at least about 70% or at least about 75% or at least about 80% or at least about 85% or at least about 90% or at least about 95% or effectively abolished to an undetectable level.

Antisense techniques may be used to reduce gene expression in wheat cells. The term “antisense RNA” shall be taken to mean an RNA molecule that is complementary to at least a portion of a specific mRNA molecule and capable of reducing expression of the gene encoding the mRNA, preferably an SSIIa gene. Such reduction typically occurs in a sequence-dependent manner and is thought to occur by interfering with a post-transcriptional event such as mRNA transport from nucleus to cytoplasm, mRNA stability or inhibition of translation. The use of antisense methods is well known in the art (see for example, Hartmann and Endres, 1999).

As used herein, “artificially introduced dsRNA molecule” refers to the introduction of double-stranded RNA (dsRNA) molecule, which preferably is synthesised in the wheat cell by transcription from a chimeric gene encoding such dsRNA molecule. RNA interference (RNAi) is particularly useful for specifically reducing the expression of a gene or inhibiting the production of a particular protein, also in wheat (see, for example, Regina et al., 2006). This technology relies on the presence of dsRNA molecules that contain a sequence that is essentially identical to the mRNA of the gene of interest or part thereof, and its complement, thereby forming a dsRNA. Conveniently, the dsRNA can be produced from a single promoter in the host cell, where the sense and anti-sense sequences are transcribed to produce a hairpin RNA in which the sense and anti-sense sequences hybridize to form the dsRNA region with a related (to a SSIIa gene) or unrelated sequence forming a loop structure, so the hairpin RNA comprises a stem-loop structure. The design and production of suitable dsRNA molecules for the present invention is well within the capacity of a person skilled in the art, particularly considering Waterhouse et al., 1998; Smith et al., 2000; WO 99/32619; WO 99/53050; WO 99/49029; and WO 01/34815.

The DNA encoding the dsRNA typically comprises both sense and antisense sequences arranged as an inverted repeat. In a preferred embodiment, the sense and antisense sequences are separated by a spacer region which may (or may not) comprise an intron which, when transcribed into RNA, is spliced out. This arrangement has been shown to result in a higher efficiency of gene silencing (Smith et al., 2000). The double-stranded region may comprise one or two RNA molecules, transcribed from either one DNA region or two. The dsRNA may be classified as long hpRNA, having long, sense and antisense regions which can be largely complementary, but need not be entirely complementary (typically larger than about 200 bp, for example between 200 and 1000 bp). A hpRNA can also be rather small with the double-stranded portion ranging in size from about 30 to about 42 bp, but not much longer than 94 bp (see WO04/073390). The presence of the double stranded RNA region is thought to trigger a response from an endogenous plant system that destroys both the double stranded RNA and also the homologous RNA transcript from the target plant gene(s), efficiently reducing or eliminating the activity of the target gene.

The length of the sense and antisense sequences that hybridise should each be at least 19 or at least 21 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The longer the sequence, the less stringent the requirement for the overall sequence identity. The RNA molecule may of course comprise unrelated sequences which may function to stabilize the molecule. The promoter used to express the dsRNA-forming construct may be any type of promoter that is expressed in the cells which express the target gene, preferably a promoter which is preferentially expressed in the endosperm of the developing wheat grain relative to non-grain tissues of the wheat plant. When the target gene is SSIIa or other gene expressed selectively in the endosperm, an endosperm specific promoter is preferred which is not expressed in leaf or stem tissues, so as to not affect expression of the target gene(s) in other tissues.

As used herein, “silencing RNAs” are RNA molecules that have 21 to 24 contiguous nucleotides that are complementary to a region of the mRNA transcribed from the target gene, preferably SSIIa. The sequence of the 21 to 24 nucleotides is preferably fully complementary to a sequence of 21 to 24 contiguous nucleotides of the mRNA i.e. identical to the complement of the 21 to 24 nucleotides of the region of the mRNA. However, miRNA sequences which have up to five mismatches in region of the mRNA may also be used (Palatnik et al., 2003), and basepairing may involve one or two G-U basepairs. When not all of the 21 to 24 nucleotides of the silencing RNA are able to basepair with the mRNA, it is preferred that there are only one or two mismatches between the 21 to 24 nucleotides of the silencing RNA and the region of the mRNA. With respect to the miRNAs, it is preferred that any mismatches, up to the maximum of five, are found towards the 3′ end of the miRNA. In a preferred embodiment, there are not more than one or two mismatches between the sequences of the silencing RNA and its target mRNA.

Silencing RNAs derive from longer RNA molecules that are encoded by the chimeric DNAs of the invention. The longer RNA molecules, also referred to herein as “precursor RNAs”, are the initial products produced by transcription from the chimeric DNAs in the wheat cells and have partially double-stranded character formed by intra-molecular basepairing between complementary regions. The precursor RNAs are processed by a specialized class of RNAses, commonly called “Dicer(s)”, into the silencing RNAs, typically of 21 to 24 nucleotides long. Silencing RNAs as used herein include short interfering RNAs (siRNAs) and microRNAs (miRNAs), which differ in their biosynthesis. SiRNAs derive from fully or partially double-stranded RNAs having at least 21 contiguous basepairs, including possible G-U basepairs, without mismatches or non-basepaired nucleotides bulging out from the double-stranded region. These double-stranded RNAs are formed from either a single, self-complementary transcript which forms by folding back on itself and forming a stem-loop structure, referred to herein as a “hairpin RNA”, or from two separate RNAs which are at least partly complementary and that hybridize to form a double-stranded RNA region. MiRNAs are produced by processing of longer, single-stranded transcripts that include complementary regions that are not fully complementary and so form an imperfectly basepaired structure, so having mismatched or non-basepaired nucleotides within the partly double-stranded structure. The basepaired structure may also include G-U basepairs. Processing of the precursor RNAs to form miRNAs leads to the preferential accumulation of one or more distinct, small RNAs each having a specific sequence, the miRNA(s). They are derived from one strand of the precursor RNA, typically the “antisense” strand of the precursor RNA, whereas processing of the long complementary precursor RNA to form siRNAs produces a population of siRNAs which are not uniform in sequence but correspond to many portions and from both strands of the precursor.

MiRNA precursor RNAs of the invention, also termed herein as “artificial miRNA precursors”, are typically derived from naturally occurring miRNA precursors by altering the nucleotide sequence of the miRNA portion of the naturally-occurring precursor so that it is complementary, preferably fully complementary, to the 21 to 24 nucleotide region of the target mRNA, and altering the nucleotide sequence of the complementary region of the miRNA precursor that basepairs to the miRNA sequence to maintain basepairing. The remainder of the miRNA precursor RNA may be unaltered and so have the same sequence as the naturally occurring miRNA precursor, or it may also be altered in sequence by nucleotide substitutions, nucleotide insertions, or preferably nucleotide deletions, or any combination thereof. The remainder of the miRNA precursor RNA is thought to be involved in recognition of the structure by the Dicer enzyme called Dicer-like 1 (DCL1), and therefore it is preferred that few if any changes are made to the remainder of the structure. For example, basepaired nucleotides may be substituted for other basepaired nucleotides without major change to the overall structure. The naturally occurring miRNA precursor from which the artificial miRNA precursor of the invention is derived may be from wheat, another plant such as another cereal plant, or from non-plant sources. Examples of such precursor RNAs are the rice mi395 precursor, the Arabidopsis mi159b precursor, or the mi172 precursor. The use of artificial miRNAs have been demonstrated in plants, for example Alvarez et al., 2006; Parizotto et al., 2004; Schwab et al., 2006.

Another molecular biological approach that may be used to down-regulate endogenous gene expression is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the “sense orientation” with respect to a promoter for its expression, which as used herein refers to the same orientation as transcription and translation (if it occurs) of the sequence relative to the sequence in the target gene. The size of the sense fragment, its correspondence to target gene regions, and its degree of homology to the target gene are as for the antisense sequences described above. In some instances the additional copy of the gene sequence interferes with the expression of the target plant gene. Reference is made to Patent specification WO 97/20936 and European patent specification 0465572 for methods of implementing co-suppression approaches.

Any of these technologies for reducing gene expression can be used to coordinately reduce the activity of multiple genes. For example, one RNA molecule can be targeted against a family of related genes by targeting a region of the genes which is in common. Alternatively, unrelated genes may be targeted by including multiple regions in one RNA molecule, each region targeting a different gene. This can readily be done by fusing the multiple regions under the control of a single promoter.

A number of techniques are available for the introduction of nucleic acid molecules into a wheat cell, well known to workers in the art. The term “transformation” as used herein means alteration of the genotype of a cell, for example a bacterium or a plant, particularly a wheat plant, by the introduction of a foreign or exogenous nucleic acid. By “transformant” is meant an organism so altered. Introduction of DNA into a wheat plant by crossing parental plants or by mutagenesis per se is not included in transformation. The nucleic acid molecule may be replicated as an extrachromosomal element or is preferably stably integrated into the genome of the plant. By “genome” is meant the total inherited genetic complement of the cell, plant or plant part, and includes chromosomal DNA, plastid DNA, mitochondrial DNA and extrachromosomal DNA molecules. In an embodiment, a transgene is integrated in the wheat nuclear genome which in hexaploid wheat includes the A, B and D subgenomes, herein referred to as the A, B and D “genomes”.

The most commonly used methods to produce fertile, transgenic wheat plants comprise two steps: the delivery of DNA into regenerable wheat cells and plant regeneration through in vitro tissue culture. Two methods are commonly used to deliver the DNA: T-DNA transfer using Agrobacterium tumefaciens or related bacteria and direct introduction of DNA via particle bombardment, although other methods have been used to integrate DNA sequences into wheat or other cereals. It will be apparent to the skilled person that the particular choice of a transformation system to introduce a nucleic acid construct into plant cells is not essential to or a limitation of the invention, provided it achieves an acceptable level of nucleic acid transfer. Such techniques for wheat are well known in the art.

Transformed wheat plants can be produced by introducing a nucleic acid construct according to the invention into a recipient cell and growing a new plant that comprises and expresses a polynucleotide according to the invention. The process of growing a new plant from a transformed cell which is in cell culture is referred to herein as “regeneration”. Regenerable wheat cells include cells of mature embryos, meristematic tissue such as the mesophyll cells of the leaf base, or preferably from the scutella of immature embryos, obtained 12-20 days post-anthesis, or callus derived from any of these. The most commonly used route to recover regenerated wheat plants is somatic embryogenesis using media such as MS-agar supplemented with an auxin such as 2,4-D and a low level of cytokinin, see Sparks and Jones, 2004).

Agrobacterium-mediated transformation of wheat may be performed by the methods of Cheng et al., 1997; Weir et al., 2001; Kanna and Daggard, 2003 or Wu et al., 2003. Any Agrobacterium strain with sufficient virulence may be used, preferably strains having additional virulence gene functions such as LBA4404, AGL0 or AGL1 (Lazo et al., 1991) or versions of C58. Bacteria related to Agrobacterium may also be used. The DNA that is transferred (T-DNA) from the Agrobacterium to the recipient wheat cells is comprised in a genetic construct (chimeric plasmid) that contains one or two border regions of a T-DNA region of a wild-type Ti plasmid flanking the nucleic acid to be transferred. The genetic construct may contain two or more T-DNAs, for example where one T-DNA contains the gene of interest and a second T-DNA contains a selectable marker gene, providing for independent insertion of the two T-DNAs and possible segregation of the selectable marker gene away from the transgene of interest. The T-DNA vector is preferably a “super-binary” plasmid as known in the art.

Any wheat type that is regenerable may be used; varieties Bob White, Fielder, Veery-5, Cadenza and Florida have been reported with success. Transformation events in one of these more readily regenerable varieties may be transferred to any other wheat cultivars including elite varieties by standard backcrossing. Other methods involving the use of Agrobacterium include: co-cultivation of Agrobacterium with cultured isolated protoplasts; transformation of seeds, apices or meristems with Agrobacterium, or inoculation in planta such as the floral-dip method for Arabidopsis as described by Bechtold et al., 1993.

Another method commonly used for introducing the nucleic acid construct into a plant cell is high velocity biolistic penetration by small particles (also known as particle bombardment or microprojectile bombardment) with the nucleic acid to be introduced contained either within the matrix of small beads or particles, or on the surface thereof as, for example described by Klein et al., 1987.

Preferred selectable marker genes for use in the transformation of wheat include the Streptomyces hygroscopicus bar gene or pat gene in conjunction with selection using the herbicide glufosinate ammonium, the hpt gene in conjunction with the antibiotic hygromycin, or the nptII gene with kanamycin or G418. Alternatively, positively selectable markers such as the manA gene encoding phosphomannose isomerase (PMI) with the sugar mannose-6-phosphate as sole C source may be used.

The present invention is further described by the following non-limiting Examples.

### Example 1: Materials and Methods

Plant Materials.

Three wheat cultivars which each comprised single null mutations in an SSIIa gene on the A, B or D genomes were kindly provided by Dr M Yamamori, National Institute of Agrobiological Resources, Tsukuba, Japan. They were Chousen 57 (C57) comprising a null mutation in SSIIa-A and therefore lacking SSIIa-A polypeptide (SGP-A1), Kanto 79 (K79) comprising a null mutation in SSIIa-B and therefore lacking SSIIa-B polypeptide (SGP-B1), and Turkey 116 (T116) comprising a null mutation in SSIIa-D and therefore lacking the SGP-D1 polypeptide (Yamamori et al., 2000).

Chinese Spring (CS) nullisomic/tetrasomic lines for homologous group seven chromosomes, designated N7AT7D, N7BT7D and N7DT7B (Sears and Miller, 1985) were kindly supplied by Dr E. Lagudah (CSIRO Agriculture, Canberra, Australia).

Wheat plants including C57, K79, T116, three Australian wheat cultivars Sunco, EGA Hume and Westonia were grown at the CSIRO Agriculture, Canberra, in a glasshouse with natural light and at temperatures of 18° C. (night) and 24° C. (day). Mature grain of each line was harvested and air-dried to a moisture content of approximately 9%. Unless otherwise specified, 5 g of dried grain per line was milled using a Udy Cyclone mill (Fort Collins, Colo., USA) with 0.5 mm mesh scieen to generate wholemeal flour or using a Brabender Quadrumat Junior mill (Brabender© GmbH & Co. KG, Duisburg, Germany) to obtain white flour.

For analysis of proteins and starch in developing grain and mature grain as described in Example 12′, mutant and wild-type wheat plants (ssIIa and SSIIa, respectively) were obtained from a double haploid population reported by Konik-Rose et al. (2007). Twenty to forty developing endosperms were collected at 15 days post anthesis (DPA) in tubes on dry ice and stored at −80° C. for the analyses of RNA, soluble proteins and starch granule bound proteins as described in Example 12. For analysis of proteins and starch properties in grain, grain was harvested at maturity from glasshouse or field-grown plants.

DNA Analysis of Wheat Plants.

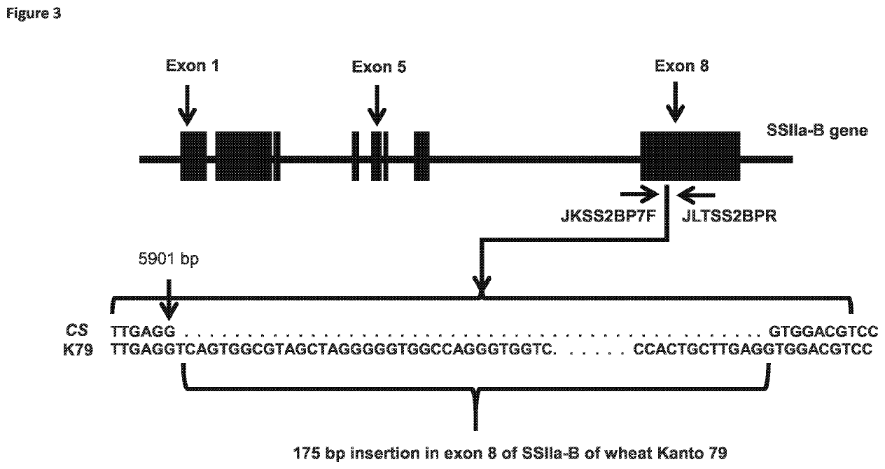

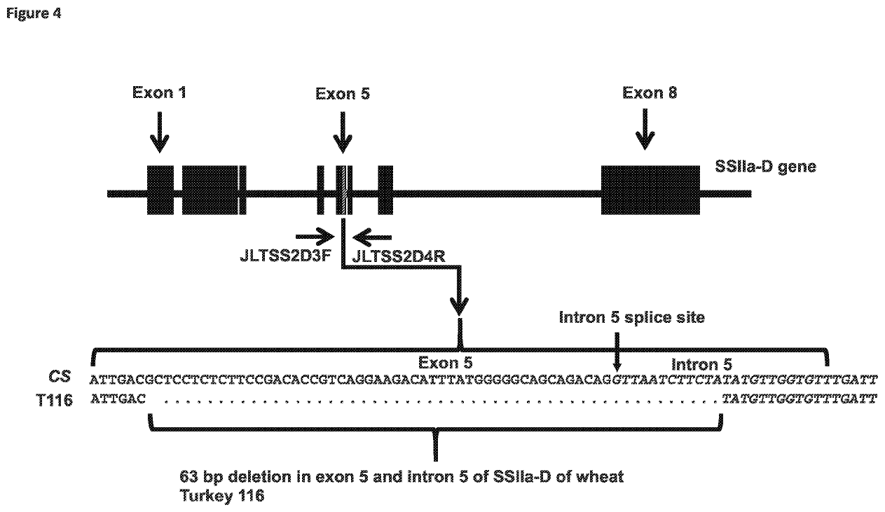

For detecting the presence or absence of mutant ssIIa or wild-type SSIIa alleles by PCR on genomic DNA samples, young leaves were harvested from plants and genomic DNA extracted using a Fast DNA Kit (BIO101 system, Q-BIO gene). For marker-assisted breeding, primer pairs JKSS2AP1F (5′-TGCGTTTACCCCACAGAGCA CA-3′ (SEQ ID NO:15) located between nucleotides 91 and 113 of nucleotide sequence Accession No. AB201445) and JKSS2AP2R (5′-TGCCAAAGGTCCGGAATCATGG-3′ (SEQ ID NO:16) located between nucleotides 1225 and 1246 of AB201445) were used for the A genome SSIIa gene (FIG. 2); primers JKSS2BP7F (5′-GCGGACCAGGTTGTCGTC-3′ (SEQ ID NO:17) located between nucleotides 5978 and 5995 of nucleotide sequence Accession No. AB201446) and JLTSS2BPR1 (5′ CTGGCTCACGATCCAGGGCATC-3′ (SEQ ID NO:18) located between nucleotides 6313 and 6335 of AB201446) for the B genome SSIIa gene (FIG. 3); and primers JTSS2D3F (5′-GTACCAAGGTATGGGGACTATGAA-3′ (SEQ ID NO:19) located between nucleotides 2369 and 2392 of nucleotide sequence Accession No. AB201447) and JTSS2D4R (5′-GTTGGAGAGATACCTCAACAGC-3′ (SEQ ID NO:20) locating between nucleotides 2774 and 2796 of AB201447) were used for the D genome SSIIa gene of wheat (FIG. 4).

The PCR reactions contained 50 ng of genomic DNA, 1.5 mM MgCl2, 0.125 mM of each dNTP, 10 pmol of primers, 0.5 M glycine betaine, 1 μl of dimethylsulphoxide (DMSO), and 1.5-3.5 U of Hot-star Taq polymerase (QIAGEN) in reaction volumes of 20 μl. The amplification reactions were conducted using a HYBAID PCR Express (Integrated Sciences) with one cycle of 95° C. for 5 min, 35 cycles of melting at 94° C. for 45 s, annealing temperature of 52° C. (A genome) or 60° C. (for B and D genome) for 30 s, and extension at 72° C. for 2 min 30 s, then 1 cycle of 72° C. for 10 min followed by cooling to 25° C. The resultant PCR fragments were separated on 1% or 2% agarose gels and visualized (UVitec) after ethidium staining. Other, standard amplifications used 59° C. for the annealing temperature but otherwise used the same PCR conditions, unless stated otherwise.

Southern blot hybridization analysis was performed on DNA from a larger scale (9 ml) extraction from lyophilized ground tissue (Stacey and Isaac, 1994). DNA samples were adjusted to 0.2 mg/ml and digested with restriction enzymes such as BainHI and EcoRI. Restriction enzyme digestion, gel electrophoresis and vacuum blotting are carried out as described by Stacey and Isaac. (1994). ³²P-labeled probes were produced from cDNA and used in hybridisation to Southern blots. Hybridising sequences were detected by autoradiography according to the method of Jolly et al. (1996).

RNA Extraction and Quantitative Real-Time PCR (qRT-PCR).

Total RNA from endosperms at 15 DPA was extracted using a NucleoSpin® RNA Plant Kit (Macherey-Nagel) and quantified using Nanodrop 1000 (Thermo Scientific). Amounts of 0.5 μg of RNA templates were used for cDNA synthesis in 50 μl reactions at 50° C. using SuperScript III reverse transcriptase (Invitrogen). The cDNA template (100 rig) was used in a 10 μl qRT-PCR reaction with the annealing temperature at 58° C. using RT-PCR primers (Table 4). As a quantitation control, a pair of primers was used which amplified a region located in an exon at the 3′ end of a tubulin gene. The amplification reactions were conducted in a Rotor-Gene 6000 (Corbett) using a Rotor-Gene™ SYBR® Green PCR Kit (QIAGEN). Comparative quantitation was analysed using amplification of the tubulin gene fragment as a reference amplification in the Real Time Rotary Analyzer Software (Corbett). For each sample, triplicates of qRT-PCR reactions were performed.

TABLE 4
QRT-PCR primers used for RNA expression determination

| | | Oligonucleotide | SEQ ID | |
|---|---|---|---|---|
| Cereal | Name | Sequence | NO | Reference |
| Barley | ZLBSSI-1RTR3 | AAGGTCTCCACCGTGTCTCGAAG | 21 | This study |
| | ZLBSSI-1RTF3 | GACGTACAGTTTGTCATGCTTGG | 22 | This study |
| | ZLBSSIIaF | CTGCTGGACAGGATATGGAAGT | 23 | This study |
| | ZLBSSIIaR | GTATCACCATAACGGAGCGACT | 24 | This study |
| | ARHv2aF1 | CAATCACTGATGGTGTAACCAAA | 25 | Regina et al., |
| | ARHv2aR3 | CCTTCATGTTGGTCAATAGCAGC | 26 | Regina et al., |
| | ARHv2bF1 | CAGAATGGACAAAGAATCATCC | 27 | Regina et al., |
| | ARHv2bR1 | GAAAATACATCCATGCCTCCATC | 28 | Regina et al., |
| Wheat | SSIFw | AGGGTACAGGGTGGGCGTTCT | 29 | Sestili et al., 2010 |
| | SSIR | GTAGGGTTGGTCCACGAAGG | 30 | Sestili et al., 2010 |
| | SSIIFw | TTCGACCCCTTCAACCACTC | 31 | Sestili et al., 2010 |
| | SSIIR | ACGTCCTCGTAGAGCTTGGC | 32 | Sestili et al., 2010 |
| | SBEIIaFw | TGACGAATCTTGGAAAATGG | 33 | Sestili et al., 2010 |
| | SBEIIaR | GGCGGCATTTATCATAACTATTG | 34 | Sestili et al., 2010 |
| | SBEIIbFw | GTAGATGCGGTCGTTTACTTGA | 35 | Sestili et al., 2010 |
| | SBEIIbR | CCAGCCACCTTCTGTTTGTT | 36 | Sestili et al., 2010 |
| Rice | JLRSSI-RTF | GGGCCTTCATGGATCAACC | 37 | Ohdan et al., 2005 |
| | JLRSSI-RTR | CCGCTTCAAGCATCCTCATC | 38 | Ohdan et al., 2005 |
| | JLRSSIIa-RTF | CTGGACAGGATCTGGAAGTGAA | 39 | This study |
| | JLRSSIIa-RTR | GGAACCTCAACAGCAGCCTTAC | 40 | This study |
| | JLRSBEIIa-RTF | GCCAATGCCAGGAAGATGA | 41 | Ohdan et al., 2005 |
| | JLRSBEIIa-RTR | GCGCAACATAGGATGGGTTT | 42 | Ohdan et al., 2005 |
| | sbe2b-f | TACGAATTCTCCAGCGGAATGAG | 43 | Nishi et al., 2001 |
| | sbe2b-r | TACGGTACCCAAGATGTACAGA | 44 | Nishi et al., 2001 |
| | α-tublin-f | GGAAATACATGGCTTGCTGCTT | 45 | Toyota et al., |
| | α-tublin-r | TCTCTTCGTCTTGATGGTTGCA | 46 | Toyota et al., |
| Common | JLRHvTaTub- | CAGGCTTGTATCCCAGGTCA | 47 | This study |
| | RTF | | | |
| | JLRHvTaTub- | GCGTAGGAGGAAAGCATGAA | 48 | This study |
| | RTR | | | |

Isolation of Soluble and Starch Granule Bound Proteins from Developing Endosperm.

Developing endosperms from developing seeds harvested at 15 DPA were homogenised and suspended in pre-chilled soluble protein extraction buffer at 1.5 μl/mg (0.25 M K₂HPO₄, pH 7.5, 0.05 M EDTA, 20% glycerol, Sigma protease inhibitor cocktail and 0.5 M DTT). The homogenate was centrifuged at 16,000 g for 15 min at 4° C. The supernatant containing soluble proteins was used for the estimation of protein concentration using Coomassie Plus Protein Assay Reagent (Bio-Rad). If desired, samples were stored at −20° C. prior to analysis. The pellets kept from the soluble protein preparation were directly treated with proteinase K after washing with water, and then starch was purified as follows. The starch granule bound proteins were prepared from purified starch according to Rahman et al., (1995) with minor alterations. Starch granules were boiled for 5-10 min in protein denaturing extraction buffer (50 mM Tris buffer, pH 6.8, 10% glycerol, 5% SDS, 5% β-mercaptoethanol, and bromophenol blue) at a ratio of 15 μl/mg starch. After centrifugation at 13,000 g for 20 min, the supernatant was used for SDS-PAGE analysis.

Isolation of Starch and Extraction of Starch Granule Bound Proteins from Mature Grains

Whole grains (100-150 mg) from each plant were ground in a ball bearing machine at a speed of 30 rpm for 30 s using a WIG-L-BUG Mixer MSD (USA). Wholemeal products were first treated with 12.5 mM NaOH, filtered through 0.5 mm nylon sieves, washed with water three times, and then incubated with 0.5 mg proteinase K in 1 nil of 50 mM phosphate buffer at 37° C. for 2 hrs. The starch pellets obtained by centrifugation at 5,000 g were suspended and washed with water for 3 times following with centrifugations after each wash. After washing with acetone, the residual starches were air-dried at 37° C. overnight. Preparation of starch granule bound proteins from mature grain starches were the same as that described earlier for developing endosperm starches.

SDS-PAGE and Gel Staining.

For the quantitation of protein contents in starch, an equal amount of starches (4 mg) were used for the extraction of starch granule bound proteins. The same volume of supernatant containing total proteins was loaded for each sample into NuPAGE Novex 4-12% Bis-Tris Gels (Life technologies). This allowed for the detection of variation in protein binding patterns from the same amount of starches. Samples containing 20 μg total proteins were used for soluble proteins. The SDS-PAGE gels were run and detected as previously described (Butardo et al. 2012).

Immunoblotting.

Anti-serum against GBSSI, SSI, SSIIa, SBEIIa and SBEIIb from previous studies are listed and enumerated in Table 5, including their antigen sources and specificities. Western blotting and detection was carried out as previously-described (Butardo et al. 2012) using the same protein standards as above.

TABLE 5
Polyclonal antibodies used to detect cereal starch synthase proteins.

| | | Antigen | | |
|---|---|---|---|---|
| Antibody | Dilution | source | Specificity | Reference |
| Anti-GBSSI | 1:3000 | wheat | barley, wheat, rice | Li et al., 1999 |
| Anti-SSI | 1:4000 | wheat | barley, wheat, rice | Rahman et al., 1995 |
| Anti-SSIIa | 1:500 | rice | barley, wheat, rice | Kosar-Hashemi et al., 2007 |
| Anti-SBEI | 1:2000 | wheat | barley, wheat, rice | Butardo et al., 2012 |
| Anti-SBEIIa | 1:2000 | wheat, rice | barley, wheat, rice | Regina et al., 2005 |
| Anti-SBEIIb | 1:3000 | wheat, rice | barley, wheat, rice | Regina et al., 2005 |

Quantitation of Protein Bands on SDS-PAGE Gels and Immunoblots.

To quantitate and compare the abundance of proteins between different genotypes, two protein bands (80 kDa and 60 kDa) in 5 μL MagicMark™ XP protein ladders (Invitrogen) were used as references. After visualising the protein bands, SDS-PAGE gels and immunoblots were scanned (Epson Perfection 2450 PHOTO; Epson America Inc., CA, USA) to image files for band intensity analysis using the Quantity One software package following the prescribed methods (Bio-Rad). Band 80 kDa was used for the quantitation of SBEIIa, SBEIIb and SSIIa, band 60 kDa for SSI and GBSSI.

Mass spectromeny.

In-gel proteolytic digestion can be done on selected protein bands from Coomassie Blue stained SDS-PAGE gels. Ion trap tandem MS can be conducted as described by Butardo et al. (2012). Proteins can be identified by the correlation of uninterrupted MS to entries in SwissProt/TREMBL, through ProteinLynx Global Server (Version 1, Micromass) (Colgrave et al. 2013).

Grain weight.

Grain was harvested from plants at maturity, which was taken as when the plants were completely yellowed. The heads were harvested and stored at 37° C. for at least two weeks to ensure complete drying, then stored at room temperature if further storage was required, and then threshed to provide mature grain. It was this grain or wholemeal obtained from it that was analysed for parameters described herein that depend on the grain weight, as well as the grain weight itself. Grain weight for each wheat line was determined from the total weight of 100 grains. The moisture content of grain was measured with a MPA FT-NIR spectrometer (BRUKER); this was typically about 9% on a weight basis for mature grain. Parameters that depend on the grain weight such as starch content, BG content, fructan content etc as described herein were calculated on a dry weight basis, assuming a 9% moisture content (w/w) if not measured by NIR.

Wheat Grain Lipid Analysis.

Total lipids from samples of wholemeal of about 300 mg were extracted with a mixture of chloroform/methanol/0.1 M KCl (at a ratio of 2:1:1, v/v/v). Fatty acid methyl esters (FAME) were prepared by incubating lipid samples in 1 N Methanolic-HCl (Supelco, Bellefonte, Pa.) at 80° C. for 2 h. TAG and polar membrane lipid pools were fractionated from total lipids by thin layer chromatography (TLC) (Silica gel 60, Merck, Germany) using a solvent mixture of hexane:diethylether:acetic acid (70:30:1, v/v/v) and individual membrane lipid classes were separated by TLC using a solvent mixture of chloroform/methanol/acetic acid/water (90/15/10/3, v/v/v/v). Authentic lipid standards were loaded and were run in separate lanes on the same plates for identification of lipid classes. Silica bands, containing individual class of lipid were used to prepare FAME as mentioned above and were analyzed by gas chromatography GC-FID 7890A (Agilent Technologies, Palo Alto, Calif., USA) that was fitted with a 30 m BPX70 column (SGE, Austin, Tex., USA) for quantifying individual fatty acids on the basis of peak area of the known amount of heptadecanoin that was added as an internal standard.

Starch Extraction.

Unless stated otherwise, starch was extracted from grain samples by first grinding grain (10 g) to wholemeal using a Cyclone mill machine (Cyclote 1093, Tecator, Sweden). Starch was isolated from the wholemeal by a protease extraction method (Morrison et al., 1984), and washed with water using 10 ml of water per gram of wholemeal, at room temperature, with removal of the tailings. The starch was then freeze-dried and weighed for analysis. Starch is also isolated on a small scale from developing wheat grain using the method of Regina et al., (2006).

Starch Content.

The total starch content of grain was assayed by AACC method 76.13, using the total starch analysis kit (K-TSTA) supplied by Megazyme (Bray, Co Wicklow, Republic of Ireland) and calculated on a weight basis as a percentage of the mature, unmilled grain weight. Subtraction of the starch weight from the total grain weight to give a total non-starch content of the grain determined whether the reduction in total weight was due to a reduction in starch content.

Amylose Content.

Unless otherwise stated, the amylose content of starch samples was determined in triplicate by the iodometric (iodine binding) method of Morrison and Laignelet (1983) with slight modifications as follows. Approximately 2 mg of starch was weighed accurately (accurate to 0.1 mg) into a 2 ml screw-capped tube fitted with a rubber washer in the lid. To remove lipid, 1 ml of 85% (v/v) methanol was mixed with the starch and the tube heated in a 65° C. water bath for 1 hour with occasional vortexing. After centrifugation at 13,000 g for 5 min, the supernatant was carefully removed and the extraction step repeated. The starch was then dried at 65° C. for 1 hour and dissolved in urea-dimethyl sulphoxide solution (UDMSO; 9 volumes of dimethyl sulphoxide to 1 volume of 6 M urea), using 1 ml of UDMSO per 2 mg of starch. The mixture was immediately vortexed vigorously and incubated in a 95° C. water bath for 1 hour with intermittent vortexing for complete dissolution of the starch. An aliquot of the starch-UDMSO solution (50 μl) was treated with 20 μl of I₂-KI reagent that contained 2 mg iodine and 20 mg potassium iodide per ml of water. The mixture was made up to 1 ml with water. The absorbance of the mixture at 620 nm was measured by transferring 200 μl to microplate and reading the absorbance using an Emax Precision Microplate Reader (Molecular Devices, USA). Standard samples containing from 0 to 100% amylose and 100% to 0% amylopectin were made from potato amylose (Sigma catalogue No. A-0512) and potato amylopectin (Sigma, catalogue No. A-8515) and treated as for the test samples. The amylose content (percentage amylose) was determined from the absorbance values using a regression equation derived from the absorbances for the standard samples.

The amylose contents of starch samples were also determined, when stated, by debranching starch samples and then measured using size-exclusion chromatography (SEC) as described previously (Butardo et al. 2012; Castro et al. 2005). By this method, the short chains resulting from amylopectin debranching were separated from the longer amylose chains and the relative amounts determined. Pullulan standards (Shodex P-82) calibrated with the Mark-Houwink-Sakaruda equation were used for the estimation of the molecular weight from the elution time. Samples were prepared and analysed in triplicate.

Analysis of the amylose/amylopectin ratio of non-debranched starches may also be carried out according to Case et al., (1998) or by an HPLC method using 90% DMSO for separating debranched starches as described by Batey and Curtin, (1996).

Resistant Starch (RS) Content.

The RS content of grain was determined in triplicate using the RS analysis kit (K-RSTARCH) supplied by Megazyme (Bray, Co Wicklow, Republic of Ireland) and calculated on a weight basis as a percentage of the starch. Instead of 100 mg sample proposed in the RS analysis kit, a reduced amount of wholemeal (40 mg) was used for each assay in this work in a 15 ml conical bottom capped tubes (Cat No: 188271, Greiner bio-one) using pro rata amounts of solutions and buffers from the kit. Standard samples containing from 0 to 20 mg/ml glucose were made from glucose (K-RSTARCH kit) and treated as for the test samples. The RS content on a weight basis and non-resistant starch content for the test samples were determined from the absorbance values using a regression equation derived from the absorbance for the standard samples. The RS content was then calculated as the weight of RS as a percentage of the weight of the total starch content.

The level of RS in food samples such as bread may also be measured in vitro as described in WO2012/058730. That method describes the sample preparation and in vitro digestion of starch in foods, as normally eaten. The method has two sections: firstly, starch in the food is hydrolysed under simulated physiological conditions; secondly, by-products are removed through washing and the RS determined after homogenization and drying of the sample. Starch quantitated at the end of the digestion treatment represented the RS content of the food.

β-Glucan (BG).

BG levels were determined in triplicate using the kit (K-BGLU) supplied by Megazyme (Bray, Co, Wicklow, Republic of Ireland).

Fructan Content.

Fructan extraction and assay were performed in 2 ml tubes or 96 well plates (2 ml well) using a modified Megazyme fructan kit (K-FRUC) assay procedure as follows. Wheat wholemeal (40 mg) was mixed with 1 ml water (80° C.) and incubated with shaking (1200 rpm) at 80° C. for 30 min. After cooling to room temperature, the tubes were centrifuged for 5 min, and 20 μl of supernatant containing fructans and other sugars removed for fructan assay. Sucrose, maltose, maltodextrins and starch in the supernatant were hydrolysed to glucose and fructose by adding 20 μl of Enzyme Solution containing sucrase, amylase and maltase from the K-FRUC kit, and incubation of the mixtures at 40° C. with shaking (1000 rpm) for 30 min. Glucose and fructose in the samples were then reduced by adding 20 μl of 10 mg/ml alkaline borohydride solution and incubation at 40° C. with shaking (1000 rpm) for 30 min. Fructans in this solution were hydrolysed with fructanases (40° C. for 30 min with shaking at 1000 rpm) to glucose and fructose. p-hydroxybenzoic acid hydrazide (PAHBAH) was added to develop the colour complex at 98° C. for 6 min. After cooling the samples, the colour complex was measured at 410 nm using a spectrophotometer and the absorbance value was converted to fructan content using a standard curve which contained from 0 to 0.27 mg/ml fructose (Megazyme, K-FRUC kit) and treated as for the test samples after hydrolysis with fructanase. The fructan content (percentage fructan) for the test samples was determined from the absorbance values using a regression equation derived from the absorbances for the standard samples.

Scaled Down Fructan Assay in Plate Format.

For a scaled down fructan assay, all of the amounts for the enzyme solutions, buffers and reagents in the Megazyme kits were scaled down 10-fold and the reactions were conducted in a 96-well plate. Wholemeal samples of 20 mg for samples with high fructan content, or 40 mg samples with lower fructan levels of about 0.5-2%, were used. Pre-wetting flours with ethanol was not necessary for fructan extraction—the wholemeal was well-dispersed in hot water by vortexing before extracting fructans. For the fructan extraction, 20 min extraction time was sufficient. The hydrolysis reactions were performed in 1.1 ml 96-well plates sealed with caps at 40° C. for 30 min with shaking at 1,000 rpm using a BioShake iQ and a 96-well adaptor (Q Instruments, Jena, Germany). In the modified K-FRUC assay, the plates after fructan hydrolysis and addition of p-hydroxybenzoic acid hydrazide (PAHBAH) were sealed with caps and tightly clamped into a custom made plate holder for colour development at 100° C. for 6 min using a water bath (WiseBath, Thermoline Scientific, Wetherill Park, NSW, Australia). Hydrolysed samples (250 μl) were transferred into a 96-well flat bottom microtitre plate (UV-Star® Microplate or PS-Microplate, Greiner Bio-One, Germany) for reading absorbance at 340 nm (for K-FRUCHK) or 410 nm (for K-FRUC) using a Multiskan Spectrum plate reader (Thermo Scientific, Finland).

Total arabinoxylan (AX).

AX was measured using 20 mg samples of wholemeal in 2 ml screw capped tubes. Each sample was mixed with 1 ml of 0.5 M sulphuric acid, vortexed, and the mixtures incubated at 99° C. with shaking (1000 rpm) for 30 min. The tubes were then cooled in ice water for 5 min. The tubes were centrifuged at 10,000 g for 5 min and supernatant (800 μl) from each tube was transferred to a 96-well plate. If desired, these plates were stored at −20° C. before further treatment. For dilution, 100 μl aliquots of supernatant were transferred to another 96-well plate (Greiner bio-one masterblock) and 900 μl Milli Q water was added to each well. The diluted supernatant (100 μl) was transferred to an assay plate (BioRad Titre tube Microtubes Racked, catalog #223-9390). For making xylose standards, 100 μl of standard solutions were made having concentrations at 30, 50, 75, 100, 150 and 200 μg/ml using a 2 mg/ml stock solution (Sigma, catalog No. X-3877). 0.5 ml of freshly made phloroglucinol reagent (PGR, see below) was added to each well. The plates were sealed with MicroCap strips (National Scientific, TN3346-08C), clamped, and incubated at 100° C. for 25 min in a fume hood. The samples were then mixed well by inverting the plates. After that, 200 μl samples were transferred to a UV star plate in a fume hood. The absorbance of each sample was measured at 510 and 552 nm using a plate spectrophotometer eg Thermo multiscan.

The phloroglucinol reagent (PGR) was prepared freshly for each assay in a fumehood. For this, two solutions were made separately and then mixed. Solution 1 was made by dissolving 0.6 g Phloroglucinol (Sigma, catalog No. 7933) in 2.4 ml absolute ethanol for a few minutes in a 50 ml tube. Solution 2 comprised 55 ml glacial acetic acid with 1.1 ml concentrated hydrochloric acid (HCl) added slowly into the acetic acid. Solution 1 was transferred into a 250 ml bottle. The 50 ml tube containing residues of Solution 1 was then rinsed with Solution 2 to quantitatively transfer all of the Solution 1, and then all of Solution 2 mixed with the Solution 1 in the bottle. Finally, 0.6 ml glucose solution (70 mg/ml in Milli Q water) was added into the mixture of Solutions 1 and 2.

Cellulose Content.

Cellulose assay was performed in 2 ml tubes or 96 well plates (2 ml well) using 50 mg samples of wholemeal. Lignin, hemicellulose and solubilised starch in the wholemeal were removed by adding 600 μl acetic nitric reagent (10:1 (v/v) mixture of 80% acetic acid: 70% nitric acid) and incubation of the mixtures at 99° C. with shaking (1000 rpm) for 1 hr. After cooling, the samples were centrifuged at maximum speed for 5 min and supernatants were discarded. Each pellet was washed with 1 ml water, centrifuged at maximum speed for 5 min and each supernatant discarded. For solubilising crystalline cellulose in each pellet, 1 ml of 72% H₂SO₄ was added to each sample tube. Samples were diluted based on the estimated cellulose content and cellulose standards. For developing colour, 100 μl of anthrone reagent (0.2% anthrone in 72% H₂SO₄) was added to each sample tube, and the mixtures incubated at 98° C. for 10 min. The absorbance of the treated samples was read at 620 nm using a spectrophotometer and the cellulose content was calculated by reference to a standard curve which used 0 to 0.75 mg/ml cellulose (Sigma: catalog No. G-6413).

Chain Length Distribution Analysis.

Determination of the chain length distribution of amylopectin was conducted by fluorescence-activated capillary electrophoresis (FACE) after debranching of the starch samples using a capillary electrophoresis unit according to Morell et al., (1998). Samples were prepared as previously described (O'Shea and Morell, 1996).

Starch Gelatinisation.

The gelatinisation temperature profiles of starch samples were measured in a Pyris 1 differential scanning calorimeter (Perkin Elmer, Norwalk Conn., USA). The viscosity of starch solutions was measured on a Rapid-Visco-Analyser (RVA, Newport Scientific Pty Ltd, Warriewood, Sydney), for example using conditions as reported by Batey et al., (1997). The parameters measured included peak viscosity (the maximum hot paste viscosity), holding strength, final viscosity and pasting temperature. The swelling volume of flour or starch was determined according to the method of Konik-Rose et al., (2001). The uptake of water was measured by weighing the sample prior to and after mixing the flour or starch sample in water at defined temperatures and following collection of the gelatinized material.

Starch Granule Morphology.

Starch granule morphology was examined by microscopy. Purified starch granule suspensions in water were examined under both normal and polarized light using a Leica-DMR compound microscope to determine the starch granule morphology. Scanning electron microscopy was carried out using a Joel JSM 35C instrument. Purified starches were sputter-coated with gold and scanned at 15 kV at room temperature.

Analysis of Protein Expression in Endosperm.

Specific expression of SBEI, SBEIIa and SBEIIb proteins in endosperm, in particular the level of expression or accumulation of these proteins, was analysed by Western blot procedures. Endosperm was dissected away from all maternal tissues and samples of approximately 0.2 mg were homogenized in 600 μl of 50 mM potassium phosphate buffer (42 mM K2HPO₄ and 8 mM KH₂PO₄), pH 7.5, containing 5 mM EDTA, 20% glycerol, 5 mM DTT and 1 mM Pefabloc. The ground samples were centrifuged for 10 min at 13,000 g and the supernatant aliquoted and frozen at −80° C. until use. For total protein estimation, a BSA standard curve was set up using 0, 20, 40, 60, 80 and 100 μl aliquots of 0.25 mg/ml BSA standard. The samples (3 μl) were made up to 100 with distilled water and 1 ml of Coomassie Plus Protein reagent was added to each. The absorbance was read after 5 min at 595 nm, using the zero BSA sample from the standard curve as the blank, and the protein levels in the samples determined. Samples containing 20 μg total protein from each endosperm were run on an 8% non-denaturing polyacrylamide gel containing 0.34 M Tris-HCl (pH 8.8), acrylamide (8.0%), ammonium persulphate (0.06%) and TEMED (0.1%). Following electrophoresis, the proteins were transferred to a nitrocellulose membrane according to Morell et al., 1997 and immuno-reacted with SBEIIa, SBEIIb or SBEI specific antibodies (Table 5). Antiserum against wheat SBEIIa protein (anti-wBEIIa) was generated using a synthetic peptide having the amino acid sequence of the N-terminal sequence of mature wheat SBEIIa, AASPGKVLVPDGESDDL (SEQ ID NO: 49) (Rahman et al., 2001). Antiserum against wheat SBEIIb (anti-wBEIIb) was generated in an analogous manner using the N-terminal synthetic peptide, AGGPSGEVMI (SEQ ID NO: 50) (Regina et al., (2005). This peptide was thought to represent the N-terminal sequence of the mature SBEIIb peptide and furthermore was identical to the N-terminus of the barley SBEIIb protein (Sun et al., 1998). A polyclonal antibody against wheat SBEI was synthesised in an analogous manner using the N-terminal synthetic peptide VSAPRDYTMATAEDGV (SEQ ID NO:51) (Morell et al., 1997). Such antisera were obtained from rabbits immunised with the synthetic peptides according to standard methods.

Statistical Analyses.

Statistical analysis of the amylose data was carried out using the 16th edition of Genstat for Windows (VSN International Ltd, Herts, UK). Other data were subjected to statistical analyses (t test and one-way ANOVA, with Tukey post-test) using GraphPad Prism Version 5.01. Error bars represent Standard error of mean (SEM). The statistical significance was defined at P<0.05 and P<0.01.

### Example 2. Identification of cDNA Sequences and Genomic DNA Sequences for Wheat SSIIb and SSIIc Genes and the Encoded Polypeptides and Comparison to SSIIa

To identify genes encoding starch synthase II isoenzymes (SSIIb and SSIIc) in wheat corresponding to SSIIb and SSIIc in rice (Ohdan et al., 2005) and compare them to SSIIa, the NCBI database was searched using the cDNA sequences from rice, namely Accession No. AF419099 for SSIIa, AF395537 for SSIIb and AF383878 for SSIIc. The wheat homologs were identified as follows. For the wheat SSIIa genes, three homoeologous cDNA sequences corresponding to the SSIIa genes in wheat were identified. These were Accession No: AF155217 (SEQ ID NO:4) for SSIIa-A on the A genome, AJ269504 (SEQ ID NO:5) for SSIIa-B on the B genome and AJ269502 (SEQ ID NO:6) for SSIIa-D on the D genome. The genomic DNA nucleotide sequences corresponding to these three homoeologous cDNA sequences were, identified: Accession No. AB201445 for the SSIIa-A gene (SEQ ID NO:7), AB201446 for the SSIIa-B gene (SEQ ID NO:8) and AB201447 for the SSIIa-D gene (SEQ ID NO:9). Searching the IWGSC database, the locations of the three corresponding homoeologous genes were identified on chromosome 7AS (Traes_7AS_53CAFB43A, 7A:52346437-52346905 bp, reverse strand), 7BS (IWGSC: Chromosome 7BS, Traes_7BS_7 BEAF5EC0, 7B: 31821573-31821749 bp forward strand) and 7DS (IWGSC: Chromosome 7DS, Traes_7DS_E6C8AF743, IWGSC_CSS_7DS_scaff 3877787: 1 to 396 bp, 5137 to 5419 bp forward strand), respectively. The amino acid sequences were, respectively: Accession No: AAD53263 for SSIIa-A, CAB96627 for SSIIa-B and CAB86618 for SSIIa-D polypeptides.

When the SSIIa amino acid and corresponding nucleotide sequences were compared pairwise by BLAST over the full length sequences, the homoeologous sequences were 95-96% identical for each comparison. They were therefore readily distinguished from each other.

Two cDNA sequences were identified in the NCBI database encoding wheat SSIIb genes, which were Accession Nos. AK332724 from the A genome and EU333947 from the D genome. No corresponding genomic DNA sequences were identified in the NCBI database, however, the genomic DNA sequences were found in the IWGSC database. Three homoeologous genomic DNA sequences encoding SSIIb were identified, namely the SSIIb-A gene on chromosome 6AL (homology with IWGSC: Chromosome 6AL, Traes_6AL_AE01DC0EA, 6A: 187503905 bp to 187505233 bp, forward strand through BLAST search at EnsemblPlants website) (cDNA sequence SEQ ID NO:12), the SSIIb-B gene on chromosome 6BL (Chromosome 6BL, gene: Traes_6 BL_61D83E262, 6B:162116364-162116691 bp, reverse strand through BLAST search at EnsemblPlants website) (cDNA sequence SEQ ID NO:13) and the SSIIb-D gene on chromosome 6DL (homology with IWGSC: Chromosome 6DL, gene: Traes_6DL_19F1042C7, 6D: 147050072-147051031 bp, reverse strand through BLAST search at EnsemblPlants website) (cDNA sequence SEQ ID NO:14). One amino acid sequence (Accession No: ABY56824, SEQ ID NO:11) was identified in the NCBI database which was 100% identical with the amino acid sequences deduced from EU333947; it was therefore the SSIIb-D polypeptide sequence. A full length amino acid sequence (SEQ ID NO:10) was deduced from the nucleotide sequence of AK332724 for the SSIIb-A polypeptide which had 90% homology with ABY56824 from the D genome SSIIb. An amino acid sequence for the SSIIb-B polypeptide was deduced from the DNA fragment from IWGSC (Traes_6 BL_61 D83E262, 6B:162116364-162116691 bp, reverse strand). The full length SSIIb-A and SSIIb-D amino acid sequences were approximately 90% identical. When compared pairwise, the three corresponding cDNA nucleotide sequences were 91-95% identical. When compared to the SSIIa amino acid or nucleotide sequences, the SSIIb sequences were 71-79% identical to the corresponding SSIIa paralog. Any SSII sequences could therefore be readily identified as SSIIa or SSIIb from either the amino acid or nucleotide sequences.

For the wheat SSIIc genes, one cDNA sequence (Accession No: EU307274) was identified in the NCBI database which corresponded to a gene located on wheat chromosome 1DL (homology with IWGSC: Chromosome 1DL, gene: Traes_1DL_F667ED844, IWGSC_CSS_1DL_scaff 2205619:1950-3041 bp, forward strand through BLAST search at EnsemblPlants website), this therefore corresponded to SSIIc-D. The cDNA sequence for another SSIIc gene was identifed through searching the IWGSC database, that gene was located on chromosome 1AL (IWGSC: Chromosome 1AL, gene: Traes_1AL_729 BF3204, 1A: 68687585-68688377, forward strand through BLAST search at EnsemblPlants website). The cDNA sequence had 98% identity with the sequence from nucleotides 1679 to 2469 of Accession number: EU307274. A sequence from chromosome 1BL was also identified, which was a partial length cDNA sequence for SSIIc-B (IWGSC: Chromosome 1BL, gene: Traes_1 BL_447468 BDE, 1B: 31475067-314776087 bp, forward strand through BLAST search at EnsemblPlants website). One amino acid sequence (Accession No: ABY639) was identified in the NCBI database, which had a 100% identity with the amino acid sequence deduced from the cDNA sequence of Accession No. EU307274. Two partial length amino acid sequences were also deduced from the nucleotide sequences of the genomic DNA fragments for SSIIc from the A and B genomes. When compared pairwise, the SSIIc sequences were close to 98% identical to each other. They were quite divergent to the SSIIa sequences.

It was concluded that the SSIIa genes and SSIIa polypeptide sequences could readily be distinguished from the corresponding SSIIb and SSIIc sequences.

### Example 3. Genome Specific DNA Markers for Marker-Assisted Breeding

In order to generate triple-null ssIIa mutant plants which were isogenic with wild-type plants in several different genetic backgrounds, plants of the three wheat lines C57 (null for SSIIa-A), K79 (null for SSIIa-B) and T116 (null for SSIIa-D) (Yamamori et al., 2000) were used in a series of crosses, backcrosses and inter-crosses. To detect and track the mutations, each of which are recessive, in the breeding program with molecular markers, genome-specific DNA markers based on the SSIIa gene sequences were designed and used. The specific mutations in C57, K79 and T116 SSIIa genes on the A, B and D genomes, respectively, were reported by Shimbata et al., (2005). Each of the mutations was a deletion or an insertion of DNA within the respective SSIIa genes (FIGS. 2 to 4) and therefore these mutations were ideal for designing molecular markers. A DNA marker was designed for each gene by locating a forward oligonucleotide primer upstream of each of the mutation sites and a reverse primer after each of the mutation sites, sequences are described in Example 1 “DNA analysis of wheat plants”.

These primers were used to amplify DNA fragments specific for each genome from the wild-type and the ssIIa null mutant plants. For the A genome SSIIa gene, a 1072 bp fragment was amplified from wild-type and a 778 bp fragment from the ssIIa-A null mutant gene. For the B genome SSIIa gene, a 374 bp fragment was amplified from wild-type and a 522 bp fragment from the ssIIa-B null mutant gene. For the D genome SSIIa gene, a 427 bp fragment was amplified from wild-type and a 364 bp fragment from the ssIIa-D null mutant gene. These genome-specific fragments were readily distinguished by their size by gel electrophoresis and therefore could be used as co-dominant DNA markers to detect the mutant and wild-type alleles. Other specific primer pairs based on the SSIIa gene sequence could easily be designed to provide alternative molecular markers.

### Example 4. Generation of Triple-Null ssIIa Mutants in Different Genetic Backgrounds

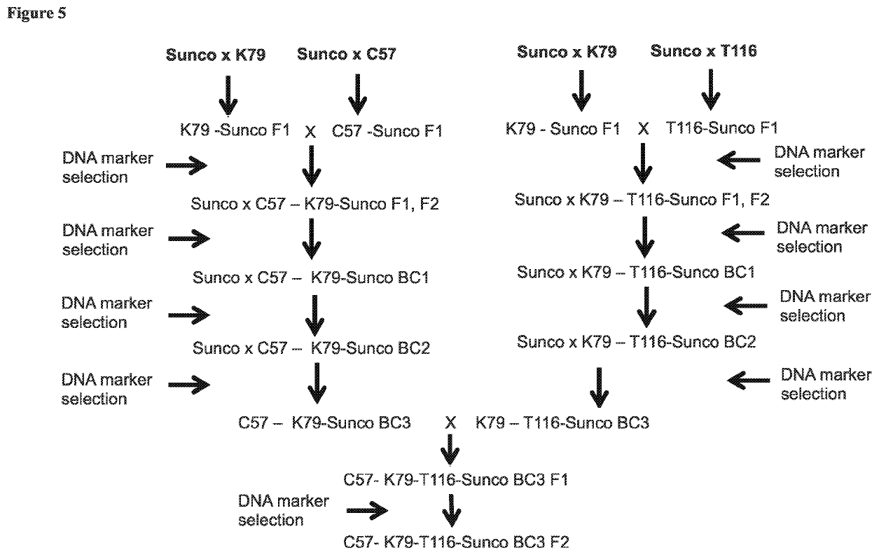

In order to generate triple-null ssIIa mutant plants which were isogenic in several different genetic backgrounds, plants of the three wheat lines C57 (null for SSIIa-A), K79 (null for SSIIa-B) and T116 (null for SSIIa-D) (Yamamori et al., 2000) were used in a series of crosses, backcrosses, intercrosses and progeny selections, shown schematically in FIGS. 5-7. The DNA markers described in Example 1 were used for screening the progeny plants in each generation, allowing for distinguishing the ssIIa null mutant alleles and the corresponding wild-type alleles in wheat varieties Sunco, EGA Hume or Westonia used as the recurrent parents. Plants of C57, K79 and T116 were first crossed with plants of wheat cultivar Sunco, using the Sunco plants as the female plants, to produce C57-Sunco F1, K79-Sunco F1 and T116-Sunco F1. Double null ssIIa mutants for C57-K79-Sunco F1 and K79-T116-Sunco F1 were then produced by crossing the single null mutants in the Sunco background followed by selfing of the progeny to produce F2 plants from the crosses. The DNA markers were used to select progeny which were heterozygous for two null ssIIa mutations. These mutants were then used in three successive back-crosses to Sunco as the recurrent parent to produce BC3 heterozygotes having two null mutations. The BC3 plants were then crossed and the progeny selfed, with selection of the triple-null ssIIa mutants (C57-K79-T116-Sunco BC3 F2) in the Sunco genetic background (FIG. 5).

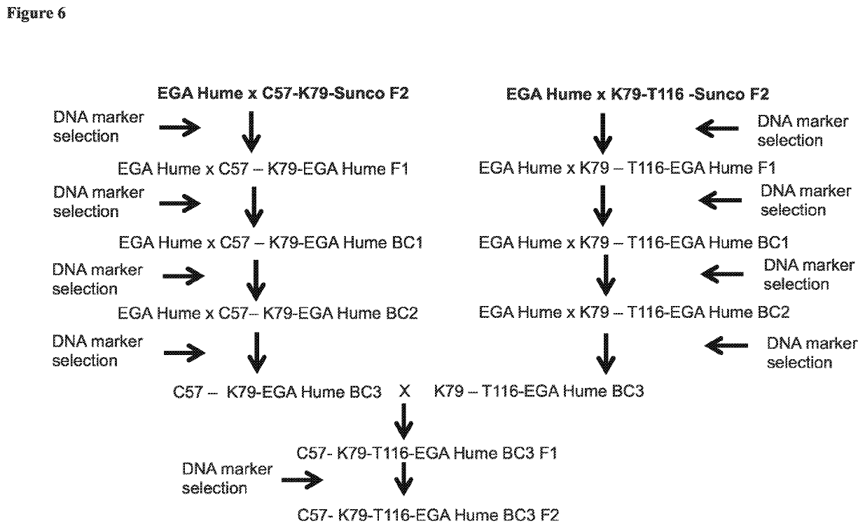

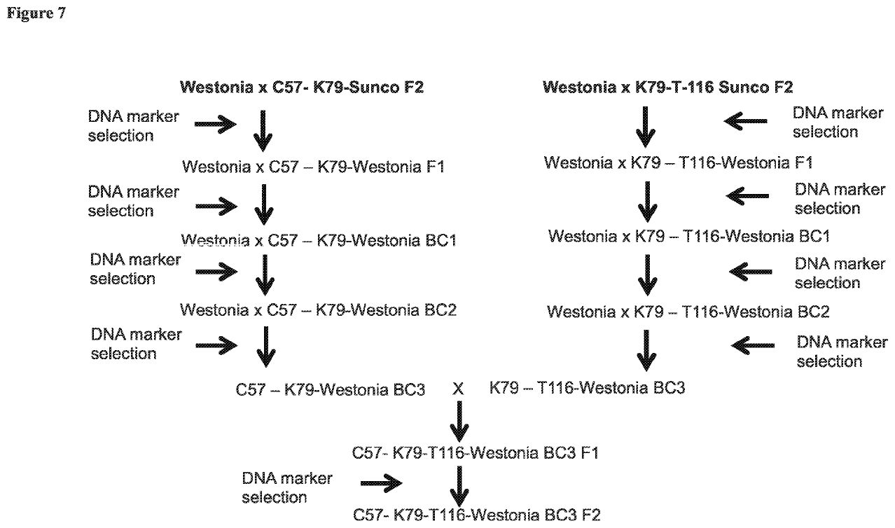

Producing BC3F8 seeds for ssIIa null mutants and wild-type wheat lines in cv. EGA Hume, Sunco and Westonia backgrounds. To produce plants and grain in two different genetic backgrounds other than Sunco, double mutants in Sunco (C57-K79-Sunco F1 or F2, K79-T116-Sunco F1 or F2) were used as pollen donors in crosses to plants of the cultivars EGA Hume and Westonia (FIGS. 6 and 7), with use of the DNA markers in each generation to detect and select the mutant alleles in the progeny. Selected double mutant ssIIa progeny were used in three successive back-crosses to EGA Hume or Westonia as recurrent parent, resulting in BC3 plants. Double mutants were crossed and selfed, producing 466 F2 progeny, from which a total of 21 triple-null ssIIa plants (designated as “abd” genotype) were selected (FIGS. 6 and 7). The 466 progeny included wild-type, single null ssIIa and double null ssIIa genotypes as well as all combinations of heterozygotes for the three SSIIa genes.

Following the production and selection of the 21 triple-null mutants covering the three genetic backgrounds, three generations of single seed descent (SSD) were performed to generate increased homozygosity in each of the three genetic backgrounds, providing the BC3F3, BC3F4 and BC3F5 generations of grain. The BC3F5 grain were further bulked up in three growing generations to produce 10 to 20 g of grain of the BC3F8 generation of each of the lines.

Triple wild-type SSIIa segregants (ABD genotype) were also generated from the crosses and selected as control lines. In each generation, the DNA markers for all three genomes were used for selecting ssIIa mutants or wild-type SSIIa alleles for each genome for each generation. Eventually, 4, 6 and 11 triple-null mutant ssIIa lines of the BC3F8 generation were generated for the EGA Hume, Sunco and Westonia genetic backgrounds, respectively, and 5 wild-type SSIIa BC3F8 lines were generated for each of the EGA Hume, Sunco and Westonia genetic backgrounds. These were grown at the same time under the same growing conditions, grain harvested at plant maturity, and the grain dried to about 9% moisture content (on a weight basis). These grain lots were analysed for various parameters including seed weight, starch content, amylose content, total dietary fibre, lipid content etc. as described below.

### Example 5. Analysis of Grain and Starch Parameters

Grain Weight.

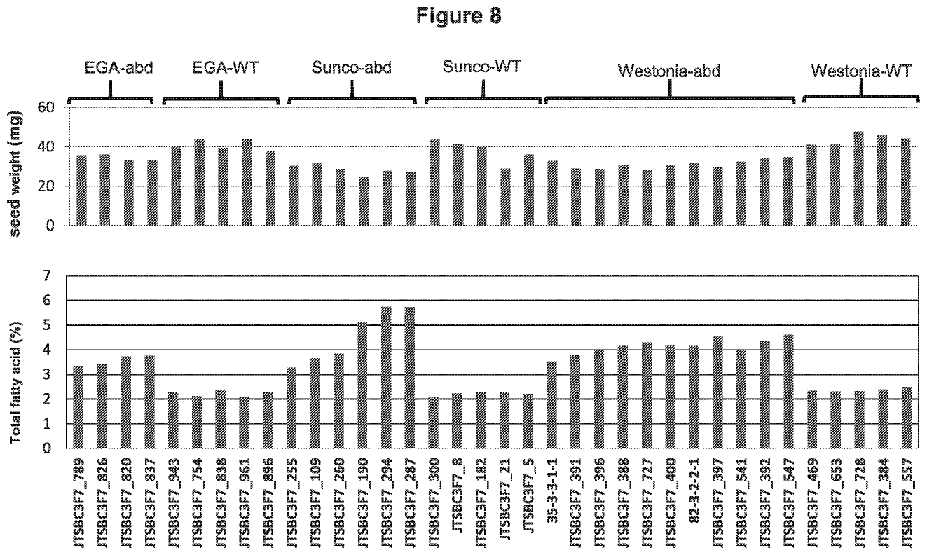

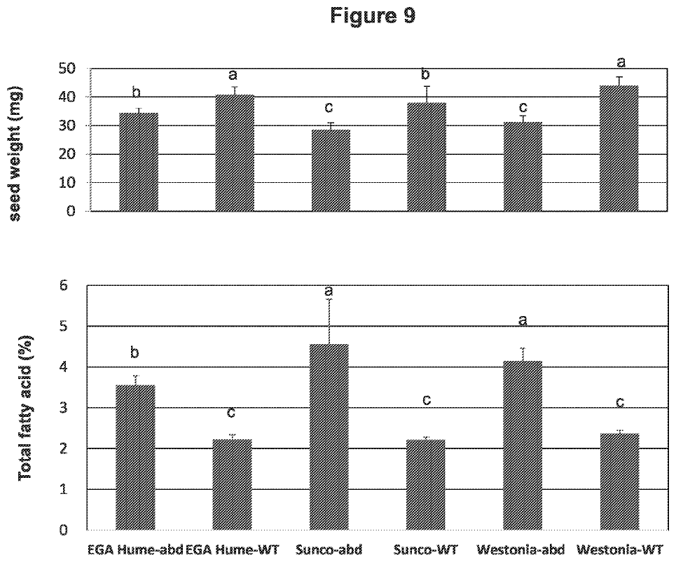

The average grain weight (mg per grain) of the triple-null ssIIa mutants and the wild-type grain in three genetic backgrounds was calculated by measuring the weight of 100 grains from each line. Average per grain weight ranged from 25 mg to 36 mg for ssIIa null mutants and 29 mg to 48 mg for the wild-type lines (Table 6, FIG. 8). The plants were grown under far from ideal growing conditions, which explained the low weights even for the wild-type controls. The mean data are shown in FIG. 9. Compared with the wild-type lines, the triple-null mutant ssIIa grain had lower grain weight, the differences being significant (P<0.05) for each genetic background including Sunco (Table 7, FIG. 9). The mutants in Sunco, EGA Hume, and Westonia had 25%, 15% and 30% lower grain weight, respectively. This was not surprising, given that SSIIa encodes a starch synthase which is involved in starch production and it was known that mutants in SSIIa produced less starch (Yamamoto et al., 2000; Konik-Rose et al., 2007).

There were no statistically significant difference between the grain weights of the Sunco and Westonia mutants. The EGA Hume mutant grain had significantly heavier grain weight than the triple-null ssIIa mutants in the other two genetic backgrounds.

Lipid Content.

The total fatty acid content (lipid content) was measured as described in Example 1. The data are shown in Table 5 and FIG. 8 for individual lines. It was noted that there were significant increases in the TPA content in the triple-null ssIIa lines compared to the wild-type. In particular grain from three Sunco mutant lines (JTSBC3F7_190, JTSBC3F7_287 and JTSBC3F7_294) had substantially increased lipid content as a percentage of grain weight.

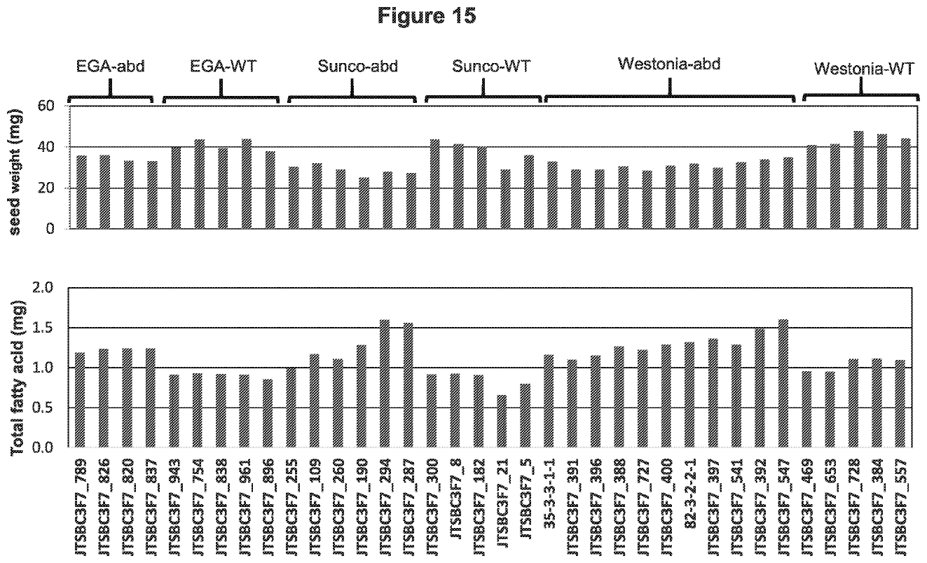

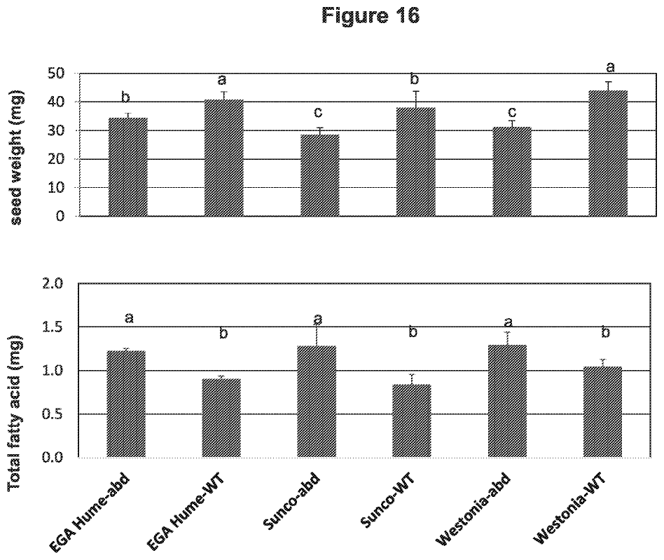

When the lipid content was calculated on a per grain basis, it was observed that the mutant lines exhibited an increased level of lipid in mg per grain (FIGS. 15 and 16).

Amylose Content.

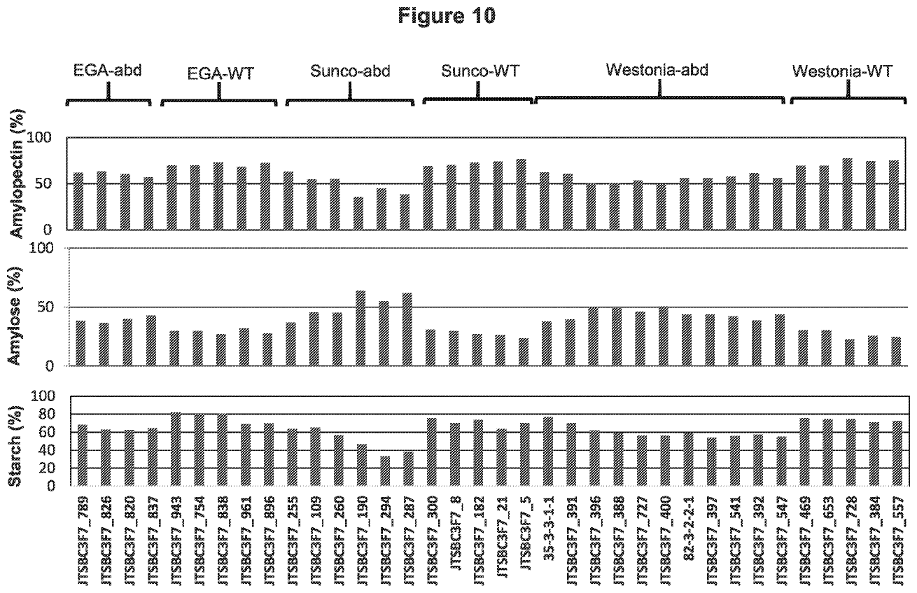

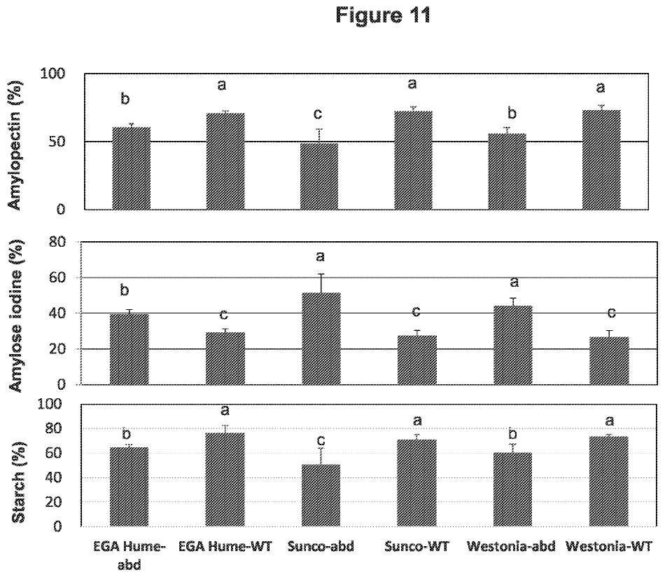

Amylose content as a proportion of the starch in the triple-null ssIIa mutants and wild-type grain in the three genetic backgrounds was measured by the iodine binding assay as described in Example 1. The data are provided in Table 6 and FIG. 10 and the means shown in Table 7 and FIG. 11 (Sunco). The amylose content for the triple-null mutants ranged from 36.6% to 64% and for the wild-type grain from 22.6% to 31.0%. Compared with the wild-type grain, the null ssIIa mutants had greater amylose content as a proportion of total starch and the differences were statistically significant. The Sunco, EGA Hume and Westonia mutant grain had 187%, 135%, and 165% of the level of amylose compared to the corresponding wild-type, respectively. Comparing the triple-null mutant grain in the three genetic backgrounds, the Sunco grain contained significantly higher proportions of amylose than the other two null mutant grain samples. There were no statistically significant differences between the EGA Hume and Westonia mutants. Likewise, there were no statistically significant differences between the three wild-type grain samples. Grain from three of the 6 Sunco triple-null mutant lines contained 61.6%, 64% and 55.1% amylose as a proportion of the starch in the grain. These values were much higher than seen previously for ssIIa mutant grain in hexaploid wheat (Yamamoto et al., 2000; Konik-Rose et al., 2007) and were therefore unexpected and surprising to the inventors.

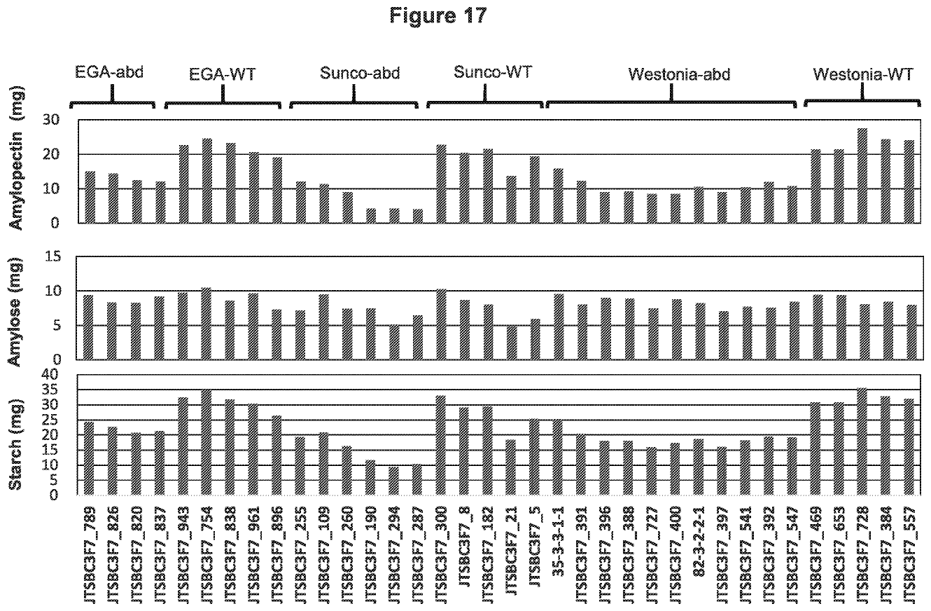

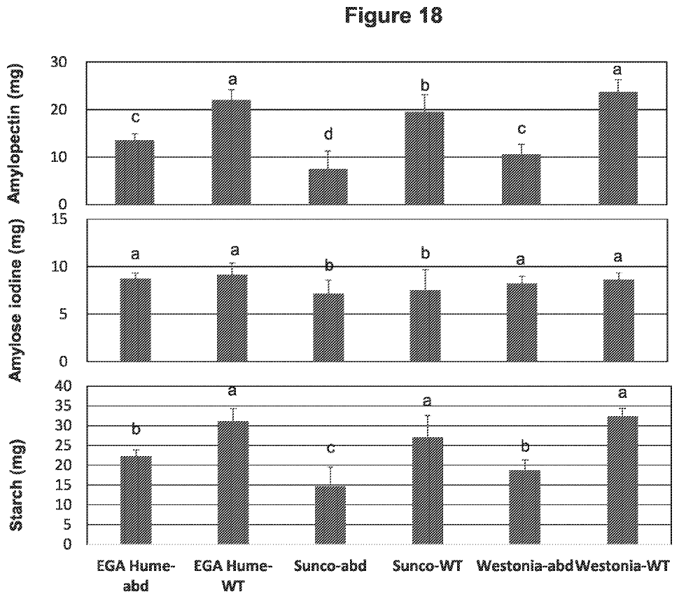

When the amylose content was calculated on a per grain basis (mg amylose per grain), it was observed that the mutant lines did not generally exhibited an increased level of amylose in mg per grain (FIGS. 17 and 18) but rather exhibited a decreased level of amylopectin and therefore decrease total starch content. The inventors concluded that this was due to the mutations in the three SSIIa genes and therefore the loss of SSIIa enzyme activity during development of the endosperm as the plants were growing.

Starch Content.

The starch content in the grain for three triple-null ssIIa mutants and three wild-type lines was measured as described in Example 1. The data are presented in Table 6 and FIG. 10 and the means are shown in Table 7 (Sunco) and FIG. 11. The starch content ranging from 30.4% to 70.0% for the triple-null ssIIa mutants and from 58.1% to 74.3% for the wild-type grain. Compared with the starch content of the wild-type lines, the EGA Hume, Sunco and Westonia mutant grain had on average 15%, 28% and 18% less starch, respectively, than their corresponding wild-type lines. These differences were statistically significant (p<0.05). The Sunco mutant grain contained significantly less starch than the ssIIa mutant grain in the other two genetic backgrounds. The starch content of the EGA Hume mutant grain was not significantly different to the Westonia grain. Grain from three Sunco mutant lines (JTSBC3F7_190, JTSBC3F7_287 and JTSBC3F7_294) had low starch content at 42.5%, 34.8% and 30.4%, respectively. These same lines also had the highest amylose content as a proportion of their starch, indicating that amylopectin synthesis was most reduced in these lines. When calculated on a mg starch per grain basis, the reduced starch content in the ssIIa mutant grain was evident (FIGS. 17 and 18), again caused by the loss of SSIIa activity.

β-Glucan Content.

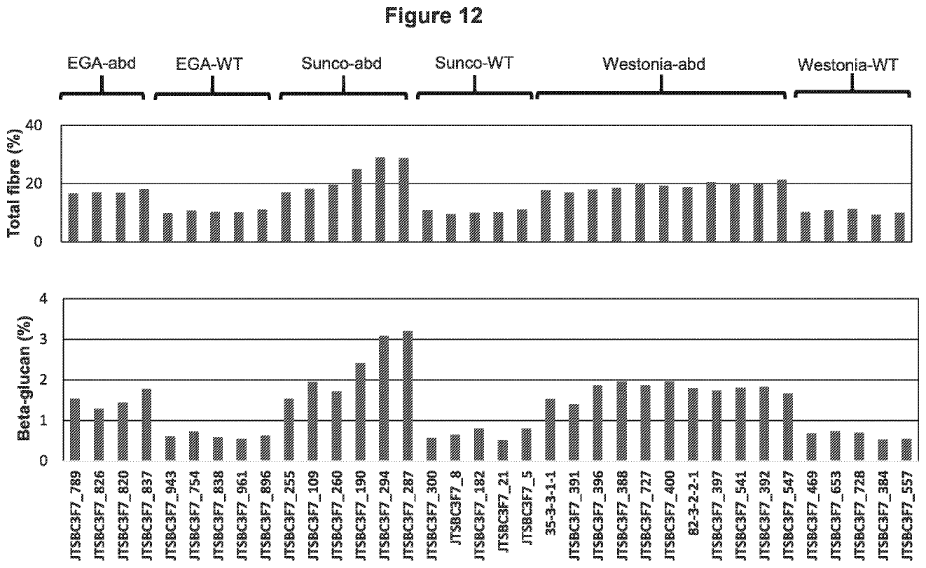

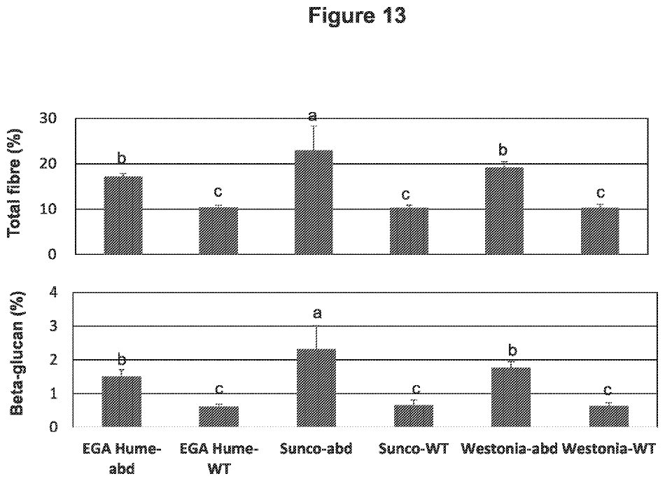

The β-glucan (BG) content of the triple-null ssIIa mutant grain and the wild-type grain was measured as described in Example 1. The data are shown in Table 6 and FIG. 12 as a percentage weight/weight of the whole grain, and the means shown in Table 7 (Sunco) and FIG. 13. The BG content ranged from 1.3% to 3.3% for the triple-null ssIIa mutants and from 0.3% to 0.8% for wild-type grain. Compared with the wild-type grain, the mutant EGA Hume, Sunco and Westonia had 144%, 245% and 177% more BG, respectively. This increase of about 1.5 to 2.5-fold was surprising to the inventors as there was no previous report of such a feature in hexaploid wheat. The Sunco mutant grain had significantly more BG than the EGA Hume and Westonia mutant grain (P<0.05). Grain of three Sunco mutant lines (JTSBC3F7_190, JTSBC3F7_287 and JTSBC3F7_294) which had the highest levels of amylose also contained the highest BG content, of 2.5%, 3.3% and 3.2%, respectively. This demonstrated the correlation between increased amylose content and increased BG content in the ssIIa mutants, each on a weight basis.

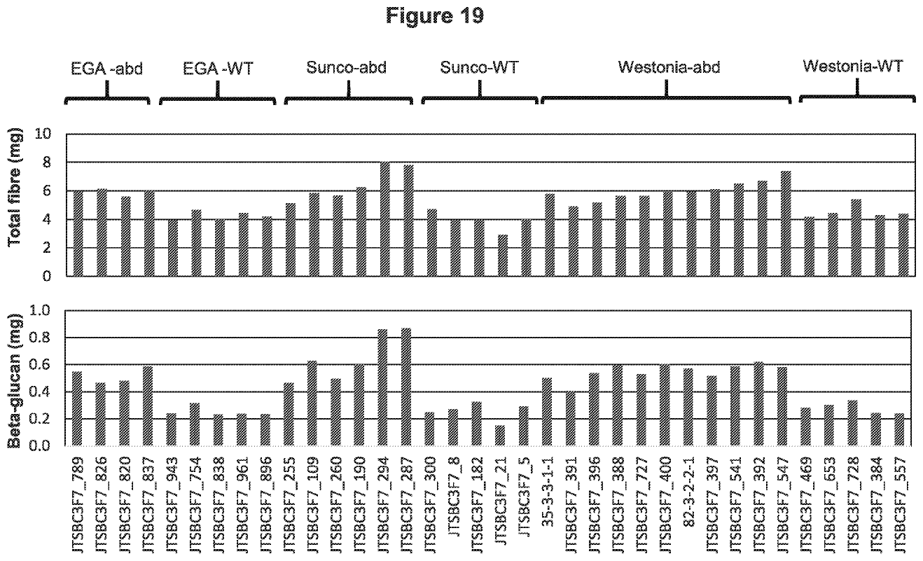

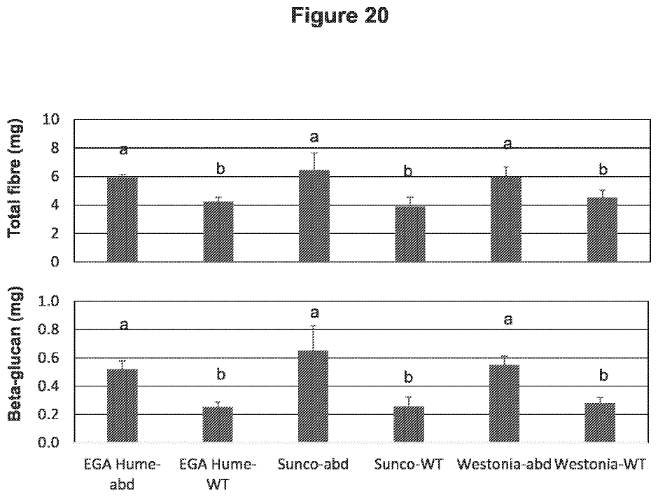

When calculated on a per grain basis, it was observed that the mutant grain had significantly increased levels of BG (FIGS. 19 and 20). The inventors considered that this was due to a diversion of carbon, coming into the grain as di-saccharides or monosaccharides, from amylopectin into BG relative to the wild-type.

Fructan Content.

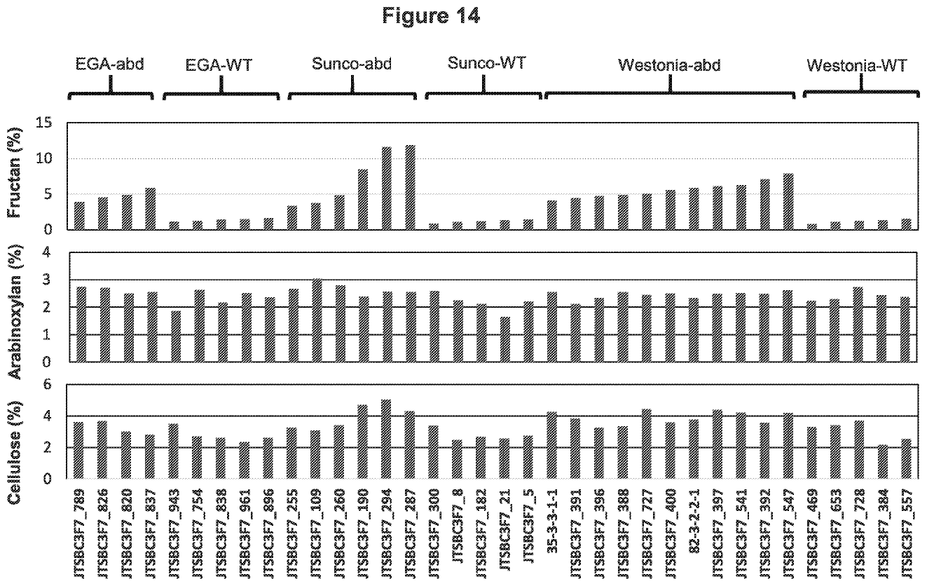

The fructan content of the triple-null ssIIa mutant grain and the wild-type grain was measured as described in Example 1. The data are shown in Table 6 and FIG. 14 as a percentage weight/weight of the whole grain, and the means shown in Table 7 for Sunco. The fructan content ranged from 3.1% to 10.8% for the triple-null ssIIa mutants and from 0.7% to 1.5% for the wild-type grain. Compared with the wild-type grain, the EGA Hume, Sunco and Westonia mutant grain had 242%, 521% and 376% more fructan, respectively. The Sunco mutant grain had significantly more fructan than the EGA Hume and Westonia mutant grain (P<0.05). The three high amylose Sunco mutant lines (JTSBC3F7_190, JTSBC3F7_287 and JTSBC3F7_294) also contained the highest fructan content of 7.7%, 10.8% and 10.5%, respectively. Such levels of fructan on a weight basis had never previously been reported in hexaploid wheat grain. This demonstrated the correlation between not only increased amylose and increased BG but also the increased fructan in the ssIIa mutants.

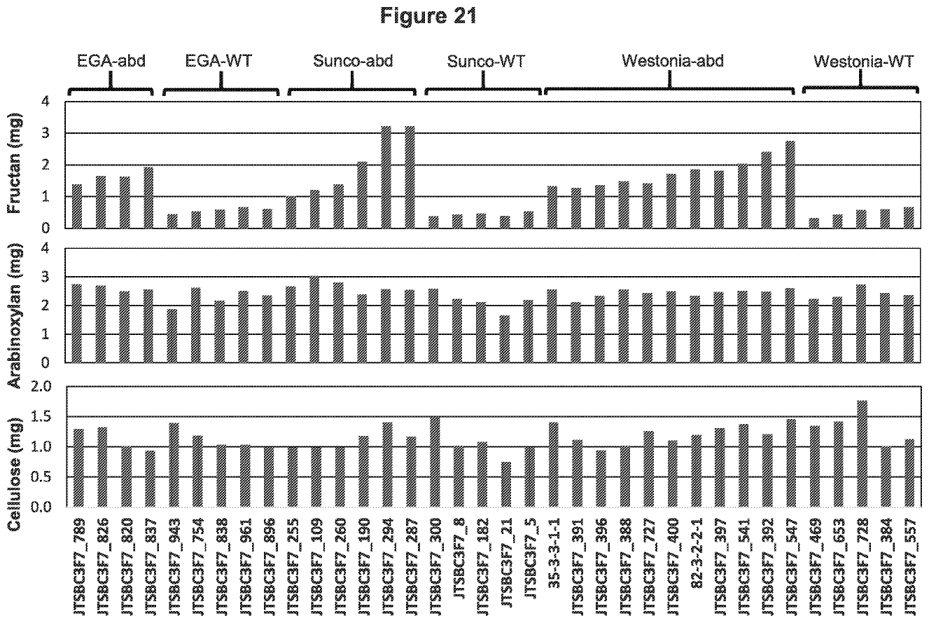

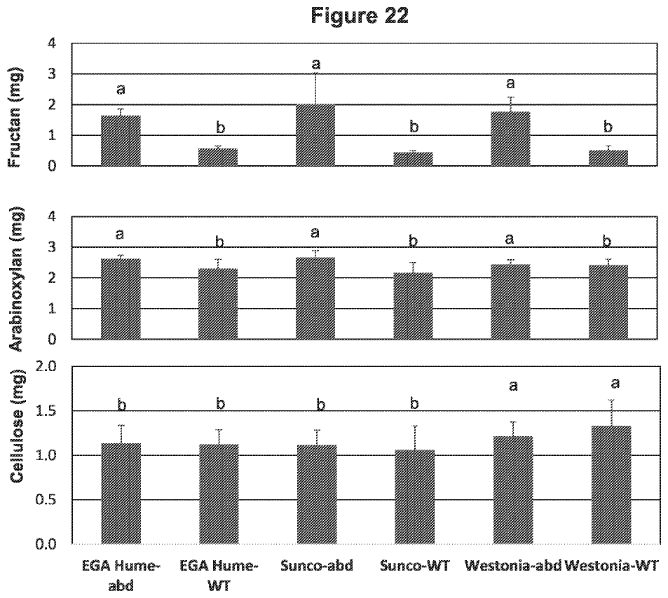

When calculated on a per grain basis, it was observed that the mutant grain had significantly increased levels of fructan (FIGS. 21 and 22). The inventors considered that this was due to a diversion of carbon, coming into the grain as di-saccharides or monosaccharides, from amylopectin into fructan relative to the wild-type during development of the endosperm.

Arabinoxylan Content.

The arabinoxylan (AX) content of the triple-null ssIIa mutant grain and the wild-type grain was measured as described in Example 1. The data are shown in Table 6 and FIG. 14 as a percentage weight/weight of the whole grain, and the means shown in Table 7 for Sunco. The AX content ranged from 6.7% to 8.8% for the triple-null ssIIa mutants and from 4.3% to 5.7% for wild-type grain. Compared with the wild-type grain, the EGA Hume, Sunco and Westonia mutant grain had 35%, 65% and 43% more AX, respectively. The Sunco mutant grain had significantly more AX than the EGA Hume and Westonia mutant grain (P<0.05). The three high amylose Sunco mutant lines (JTSBC3F7_190, JTSBC3F7_287 and JTSBC3F7_294) contained the highest AX content of 8.7%, 8.5% and 8.4%, respectively. This demonstrated the correlation between the four parameters, namely increased amylose, increased BG, increased fructan and increased AX in the ssIIa mutants. Arabinoxylan contents on a per grain basis were also significantly increased (FIGS. 21 and 22).

Cellulose Content.

The cellulose content of the triple-null ssIIa mutant grain and the wild-type grain was measured as described in Example 1. The data are shown in Table 6 and FIG. 14 as a percentage weight/weight of the whole grain, and the means shown in Table 7 for Sunco. The cellulose content ranged from 2.6% to 4.6% for the triple-null ssIIa mutants and from 2.0% to 3.4% for the wild-type grain. Compared with the wild-type lines, the mutant EGA Hume, Sunco and Westonia had 19%, 43% and 29% more cellulose, respectively. There were no significant differences in cellulose content between the three null mutants. The three high amylose Sunco lines (JTSBC3F7_190, JTSBC3F7_287 and JTSBC3F7_294) also contained high cellulose content of 4.3%, 3.9% and 4.6%, respectively. Cellulose contents were not significantly increased on a per grain basis (FIGS. 21 and 22).

Total Fibre Content.

The total fibre content for the triple-null ssIIa mutant grain and the wild-type grain was calculated as the sum of the β-glucan (BG), fructan, arabinoxylan (AX) and cellulose contents (each as a percentage of grain weight). The data are provided in Table 6 and FIG. 12 as a percentage weight/weight of the whole grain and the means are provided in Table 7 and FIG. 13. Total fibre content in the grain ranged from 15.9% to 27.5% for ssIIa null mutants and from 8.5% to 10.4% for wild-type grain. Compared with the wild-type grain, the mutants in EGA Hume, Sunco and Westonia had 68%, 125% and 88% greater total fibre content, respectively. The Sunco mutant grain had significantly more total fibre than the EGA Hume and Westonia mutant grain (P<0.05). There were no significant differences in the total fibre content between the EGA Hume and Westonia mutant grains. The three high amylose Sunco mutant lines (JTSBC3F7_190, JTSBC3F7_287 and JTSBC3F7_294) contained the highest total fibre content at 23.2%, 26.5% and 27.5%, respectively. Total fibre content was also significantly increased on a per grain basis (FIGS. 19 and 20).

Resistant Starch (RS) Content.

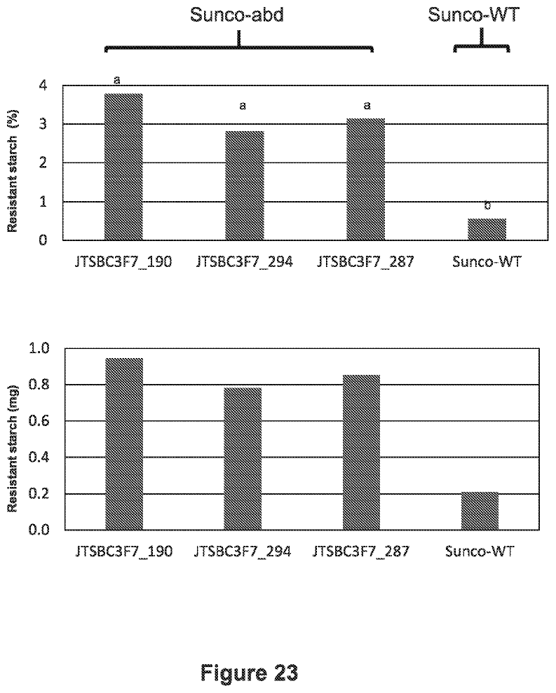

The RS content as a percentage of the starch in the triple-null ssIIa mutants and wild-type grain in the Sunco genetic background was measured using a commercial resistant starch analysis kit as described in Example 1. The data are provided in Table 8 and means shown in FIG. 23. The RS content for the triple-null mutants ranged from 1.0% to 3.8% and for the wild-type grain from 0.4% to 0.8%. Compared with the wild-type grain, the ssIIa mutants had about 5-fold greater RS content and the difference was statistically significant. Grain from three of the 6 triple-null ssIIa mutant lines in the Sunco genetic background contained 3.8%, 2.8% and 3.1% RS as a percentage of the starch in the grain (FIG. 23, upper panel). The levels of RS calculated as mg per grain were also substantially increased (FIG. 23, lower panel).

Discussion.

A number of starch properties have previously been reported to be modified in triple-null ssIIa hexaploid wheat including starch granule morphology, amylose content, amylopectin chain length distribution, crystallinity and starch gelatinization temperature, RVA and swelling power (Yamamori et al. 2000; Yamamori et al. 2006; Konik-Rose et al. 2007). The present study found that the genetic background of ssIIa null mutations had an effect on grain composition parameters including, to the surprise of the inventors, the amylose content in the starch which was increased above 45% in some lines. Three backcrossed populations were generated in different genetic backgrounds, using commercially grown wheat cultivars, and genotyped using DNA markers for the ssIIa null mutations. Four to eleven lines of the triple-null ssIIa mutants in each genetic background and five wild-type lines from each of the three breeding populations were selected and analysed.

Through the analysis of the seed weight and grain composition in each genetic background, three high amylose, high fibre, high AX and BG, and high fructan lines were identified. These three lines were from crosses with the same genetic background, namely Sunco. Wheat lines containing about 60% amylose, more than 23% total fibre and more than 7% fructan were identified and selected. Such levels have never before been reported for hexaploid wheat and were entirely unexpected to the inventors. The increases were also observed on a per grain basis. These high amylose ssIIa null mutants also contained increased BG, AX and cellulose, demonstrating the correlation of these parameters. However, these three mutant lines also showed reduced starch content and seed weight, due to substantially reduced amylopectin synthesis.

TABLE 6
Grain parameters for ssIIa triple mutants (abd) and SSIIa wild-type (ABD) wheat in three genetic backgrounds

| | | | grain | | Total | Total | Beta | | | | Lipid |
|---|---|---|---|---|---|---|---|---|---|---|---|
| Wheat | | Genetic | weight | Amylose | Starch | fibre | Glucan | Fructan | AX | Cellulose | content |
| line ID | Genotype | background | (mg) | % | % | % | % | % | % | % | % |
| SS533 | abd | EGA Hume | 35.76 | 38.4 | 67.98 | 16.67 | 1.54 | 3.87 | 7.66 | 3.60 | 3.31 |
| SS299 | abd | EGA Hume | 36.04 | 36.6 | 63.03 | 17.01 | 1.29 | 4.55 | 7.48 | 3.68 | 3.42 |
| SS412 | abd | EGA Hume | 33.23 | 39.6 | 62.57 | 16.87 | 1.45 | 4.88 | 7.52 | 3.02 | 3.74 |
| SS393 | abd | EGA Hume | 33.00 | 43.0 | 64.47 | 18.14 | 1.78 | 5.82 | 7.73 | 2.81 | 3.76 |
| SS344 | abd | Sunco | 30.25 | 37.0 | 63.75 | 16.96 | 1.54 | 3.36 | 8.81 | 3.25 | 3.27 |
| SS497 | abd | Sunco | 32.00 | 45.4 | 65.16 | 18.26 | 1.96 | 3.75 | 9.48 | 3.08 | 3.66 |
| SS435 | abd | Sunco | 28.83 | 45.0 | 56.79 | 19.62 | 1.72 | 4.79 | 9.69 | 3.42 | 3.84 |
| SS274 | abd | Sunco | 24.95 | 64.0 | 46.65 | 25.14 | 2.42 | 8.45 | 9.57 | 4.70 | 5.13 |
| SS110 | abd | Sunco | 27.81 | 55.1 | 33.37 | 28.94 | 3.09 | 11.56 | 9.24 | 5.04 | 5.74 |
| SS047 | abd | Sunco | 27.16 | 61.6 | 38.22 | 28.74 | 3.20 | 11.86 | 9.38 | 4.30 | 5.72 |
| SS104 | abd | Westonia | 32.90 | 37.7 | 76.88 | 17.62 | 1.53 | 4.05 | 7.78 | 4.26 | 3.53 |
| SS224 | abd | Westonia | 28.94 | 39.4 | 70.36 | 16.97 | 1.40 | 4.42 | 7.31 | 3.85 | 3.80 |
| SS403 | abd | Westonia | 28.84 | 49.9 | 62.34 | 17.96 | 1.87 | 4.73 | 8.11 | 3.25 | 3.99 |
| SS446 | abd | Westonia | 30.45 | 49.1 | 59.25 | 18.56 | 1.97 | 4.87 | 8.38 | 3.34 | 4.16 |
| SS066 | abd | Westonia | 28.35 | 46.4 | 56.33 | 19.91 | 1.87 | 5.00 | 8.62 | 4.43 | 4.30 |
| SS324 | abd | Westonia | 30.82 | 50.5 | 56.38 | 19.21 | 1.97 | 5.56 | 8.10 | 3.58 | 4.17 |
| SS135 | abd | Westonia | 31.76 | 43.68 | 58.92 | 18.74 | 1.80 | 5.84 | 7.36 | 3.75 | 4.15 |
| SS131 | abd | Westonia | 29.78 | 43.66 | 54.10 | 20.53 | 1.73 | 6.10 | 8.32 | 4.38 | 4.57 |
| SS306 | abd | Westonia | 32.54 | 42.35 | 56.12 | 19.97 | 1.81 | 6.25 | 7.69 | 4.22 | 3.96 |
| SS627 | abd | Westonia | 33.94 | 38.77 | 57.25 | 19.81 | 1.83 | 7.10 | 7.32 | 3.56 | 4.36 |
| SS028 | abd | Westonia | 34.78 | 43.81 | 55.22 | 21.28 | 1.67 | 7.91 | 7.51 | 4.19 | 4.61 |
| SS199 | ABD | EGA Hume | 39.68 | 30.1 | 69.90 | 9.95 | 0.61 | 1.13 | 4.70 | 3.51 | 2.30 |
| SS581 | ABD | EGA Hume | 43.59 | 29.9 | 70.10 | 10.68 | 0.72 | 1.23 | 6.02 | 2.70 | 2.13 |
| SS386 | ABD | EGA Hume | 39.40 | 27.0 | 73.00 | 10.18 | 0.59 | 1.47 | 5.51 | 2.62 | 2.34 |
| SS366 | ABD | EGA Hume | 43.81 | 31.9 | 68.10 | 10.14 | 0.55 | 1.52 | 5.73 | 2.35 | 2.09 |
| SS427 | ABD | EGA Hume | 37.72 | 27.61 | 72.39 | 11.13 | 0.63 | 1.63 | 6.25 | 2.63 | 2.26 |
| SS077 | ABD | Sunco | 43.63 | 31.0 | 69.00 | 10.77 | 0.57 | 0.85 | 5.95 | 3.41 | 2.10 |
| SS421 | ABD | Sunco | 41.35 | 29.8 | 70.20 | 9.59 | 0.66 | 1.07 | 5.41 | 2.46 | 2.23 |
| SS532 | ABD | Sunco | 39.98 | 27.1 | 72.90 | 9.97 | 0.81 | 1.18 | 5.29 | 2.69 | 2.26 |
| SS293 | ABD | Sunco | 28.93 | 26.2 | 73.80 | 10.09 | 0.52 | 1.32 | 5.68 | 2.57 | 2.27 |
| SS048 | ABD | Sunco | 36.03 | 23.44 | 76.56 | 11.12 | 0.81 | 1.46 | 6.10 | 2.75 | 2.21 |
| SS073 | ABD | Westonia | 40.88 | 30.5 | 69.50 | 10.22 | 0.68 | 0.78 | 5.46 | 3.30 | 2.34 |
| SS422 | ABD | Westonia | 41.29 | 30.5 | 69.50 | 10.79 | 0.73 | 1.07 | 5.57 | 3.42 | 2.30 |
| SS628 | ABD | Westonia | 47.72 | 22.59 | 77.41 | 11.32 | 0.70 | 1.21 | 5.71 | 3.69 | 2.32 |
| SS584 | ABD | Westonia | 46.16 | 25.66 | 74.34 | 9.30 | 0.53 | 1.31 | 5.27 | 2.19 | 2.41 |
| SS415 | ABD | Westonia | 44.06 | 24.81 | 75.19 | 10.01 | 0.55 | 1.53 | 5.37 | 2.56 | 2.49 |

TABLE 7
Mean and standard deviations (std) of grain parameters for three ssIIa triple
mutants (abd) compared to wild-type (ABD) wheat in Sunco genetic backgrounds

| | | Seed | Amylose | Amylo- | Total | Total | Beta | | Arabino- | | total |
|---|---|---|---|---|---|---|---|---|---|---|---|
| | | weight | (iodine) | pectin | Starch | fibre | Glucan | Fructan | xylan | Cellulose | lipid |
| | | (mg) | % | % | % | % | % | % | % | % | % |
| Sunco-abd | mean | 26.64 | 60.23 | 39.77 | 39.41 | 27.60 | 2.90 | 10.62 | 9.40 | 4.68 | 5.53 |
| Sunco-WT | mean | 37.98 | 27.51 | 72.49 | 70.80 | 10.31 | 0.67 | 1.17 | 5.68 | 2.78 | 2.22 |
| Sunco-abd | std | 1.50 | 4.60 | 4.60 | 6.72 | 2.14 | 0.42 | 1.89 | 0.17 | 0.37 | 0.35 |
| Sunco-WT | std | 5.77 | 2.99 | 2.99 | 4.51 | 0.62 | 0.14 | 0.24 | 0.34 | 0.37 | 0.07 |

TABLE 8
Resistant starch content of starch in the ssIIa mutant grain in the Sunco background

| | | | | | | per seed | RS per | | |
|---|---|---|---|---|---|---|---|---|---|
| wheat | | RS | | RS | | weight | seed | | |
| ID | Line name | (%) | std | (%) | std | (mg) | (mg) | mean | std |
| SS344 | JTSBC3F7_255 | 1.04 | 0.13 | 2.56 | 0.94 | 30.25 | 0.31 | 0.71 | 0.23 |
| SS497 | JTSBC3F7_109 | 2.45 | 0.05 | | | 32.00 | 0.78 | | |
| SS435 | JTSBC3F7_260 | 2.1 | 0.15 | | | 28.83 | 0.61 | | |
| SS274 | JTSBC3F7_190 | 3.79 | 0.22 | | | 24.95 | 0.95 | | |
| SS110 | JTSBC3F7_294 | 2.81 | 0.22 | | | 27.81 | 0.78 | | |
| SS047 | JTSBC3F7_287 | 3.14 | 0.49 | | | 27.16 | 0.85 | | |
| SS077wt | JTSBC3F7_300 | 0.41 | 0.18 | 0.55 | 0.16 | 43.63 | 0.18 | 0.20 | 0.04 |
| SS421wt | JTSBC3F7_008 | 0.64 | 0.11 | | | 41.35 | 0.26 | | |
| SS532wt | JTSBC3F7_182 | 0.38 | 0.11 | | | 39.98 | 0.15 | | |
| SS293wt | JTSBC3F7_021 | 0.75 | 0.63 | | | 28.93 | 0.22 | | |
| SS048wt | JTSBC3F7_005 | 0.59 | 0.5 | | | 36.03 | 0.21 | | |
| l.s.d. | | | | 0.97 | | | | 0.23 | |

*Per seed weight was the average seed weight of 100 seeds

### Example 6. Combination of SSIIa Mutations and Other Starch Synthesis Gene Mutations or Inhibitory Constructs

Wheat plants which were triple-null mutant for the three SSIIa genes as described in Example 1 were crossed with plants comprising a hairpin RNA construct designated hp-SBEIIa and having reduced starch branching enzyme II (SBEII) activity in the endosperm (Regina et al., 2006). The hp-SBEIIa parental plants showed a high amylose phenotype in grain starch (Regina et al., 2006). F1 plants were obtained and selfed to produce F2 progeny grain. Screening of 288 F2 grains was carried out by PCR using three different primer pairs described by Konik-Rose et al. (2007), each primer pair being specific for a ssIIa mutant gene from a particular genome. This resulted in the identification of one homozygous, triple-null ssIIa grain, designated YDH7, which lacked the wild-type alleles for each of the three SSIIa genes. Further testing of YDH7 DNA confirmed the presence of the hp-SBEIIa genetic construct in this grain in addition to the three mutated ssIIa genes. The grain was germinated to produce progeny plants and grain, designated herein as the YDH7 line.

In a similar fashion, plants comprising triple-null mutations in SSIIa are crossed with plants of a sbeI triple-null wheat line described in Regina et al., 2004. DNA extracted from F2 half seeds are screened for the null mutations in each of the SBEI genes using a PCR based cleaved amplified products (CAPs) marker designed from the intron 2 region of the wheat SBEI gene. The marker generates a 460 bp DNA fragment from the SBEI-D gene from the D genome, a 390 bp DNA fragment from the SBEI-B gene from the B genome and a 200 bp DNA fragment from the SBEI-A gene from the A genome. Apart from these SBEI genome-specific fragments, this CAPS marker also produces a 250 bp DNA fragment that is non-specific to the SBEI genes and is useful as an internal control in the PCR reactions. Grains are identified which lack the SBEI-specific DNA fragments amplified from the wild-type genes, indicating that they are triple-null mutants for sbeI. These triple-null grains are then tested by PCR for the presence of the ssIIa null mutations. These grains are sown to produce plants and grain which contain the combination of ssIIa and sbeI mutations, homozygous for each of the mutant alleles.

### Example 7: Analysis of Starch Granules and Starch Properties

Starch granules in wholemeal produced from triple mutant ssIIa grain were examined. The properties of the starch extracted from the wholemeal were also analysed, as follows. For these analyses, five ssIIa mutant wheat lines, designated A24, B22, B29, B63 and E24, were selected from the double haploid population of Konik-Rose et al. (2007) and analysed for starch and granule properties. Grain samples (20 g) from each line and from a corresponding wild-type line were milled to wholemeal in a Quadramat Jnr. Mill (Brabender, Cyrulla's Instruments, Sydney, Australia) after conditioning the grains to a moisture content of 14%. Starch was isolated was isolated from the wholemeal samples by a protease extraction method (Morrison et al., 1984) followed by water washing and removal of the tailings.

Changes in Starch Granule Morphology and Birefringence.

Starch granule morphology and their birefringence under polarised light were examined for granules in the wholemeal samples. Scanning electron microscopy was used to identify gross changes in starch granule size and structure. Compared to the wild-type granules, starch granules from endosperms lacking SSIIa displayed significant morphological alterations. They were highly irregular in shape and many of the A granules (>10 μm diameter) appeared to be sickle shaped. In contrast, both A and B (<10 μm diameter) starch granules in the wild-type wholemeal were smooth surfaced and spherical or ellipsoid in shape.

When observed microscopically under polarised light, wild-type starch granules typically showed a strong birefringence pattern. However, the incidence of birefringence was greatly reduced for granules from the triple-null ssIIa grain. Less than 10% of the starch granules from mutant ssIIa grains were birefringent when visualized under polarized light. In contrast, about 94% of the wild-type starch granules exhibited full birefringence. Loss of birefringence therefore correlated with the lack of SSIIa and increased amylose content.

Chain Length Distribution of Starch by FACE.

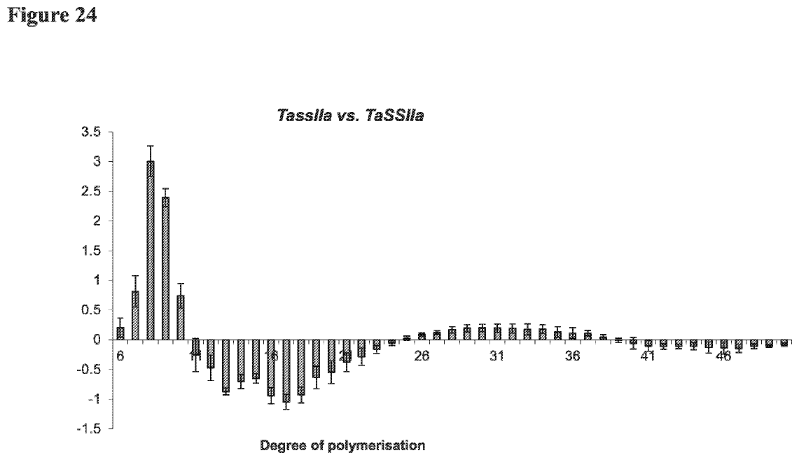

Chain length distribution of isoamylase de-branched starch was determined by fluorophore assisted carbohydrate electrophoresis (FACE) as described in Example 1. This technique provided a high resolution analysis of the distribution of chain lengths in the range from DP 1 to 50 and the relative frequency of chains of different lengths in the modified starch compared to wild-type starch. From the molar difference plot (FIG. 24) in which the normalized chain length distribution of the wild-type starch was subtracted from the normalized distribution of the triple mutant ssIIa starch, it was observed that there was a marked increase in the proportion of chain lengths of DP 7-10 and a substantial decrease in the chain lengths of DP 11-24 in starch from the triple-null ssIIa grain. There was also a slight increase in chain lengths of DP 26-36.

Molecular Weight of Amylopectin and Amylose.

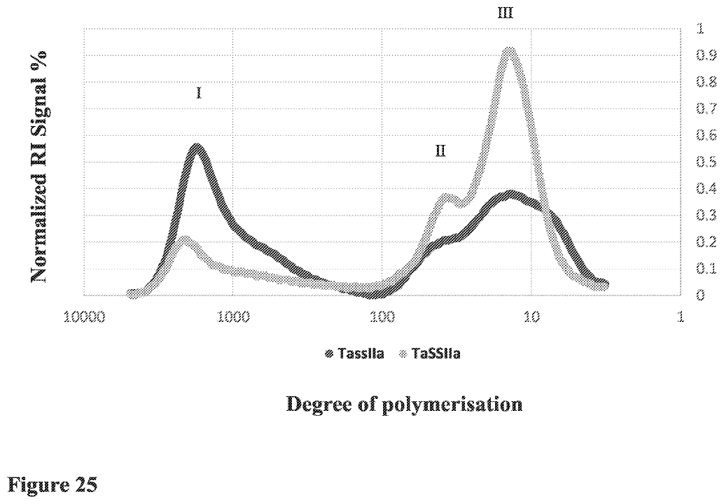

The molecular weight distribution of starch from the mutant grain was determined by size exclusion chromatography (SEC) after isoamylase debranching. The isoamylase debranching treatment cleaves each of the α-1,6-linkages in amylopectin to release the separate chains but leaves the amylose mostly untouched, therefore allowing separation of the amylose (eluting first) and the glucan chains from the amylopectin on the basis of size. FIG. 25 shows the resultant SEC traces for the debranched starches according to their degree of polymerisation. The relative amount of amylose (first peak) in the starch of the triple-null ssIIa mutant grain was substantially increased relative to the wild-type starch, and the amount of amylopectin (peak III) was much reduced compared to wild-type. The average molecular weight of the amylose in the starch of the triple-null ssIIa mutant grain appeared to be reduced compared to that of amylose in the wild-type starch, with amylose peak positions at 274 kDa versus 330 kDa for the wild-type (FIG. 25).

Starch Swelling Power (SSP).

Starch swelling power of gelatinized starch was determined following the small scale test of Konik-Rose et al., (2001) which measured the uptake of water during gelatinization of starch. The estimated value of SSP was significantly lower for starch from the triple-null ssIIa grain with a figure of 6.85 compared to starch from the wild-type at 11.79.

Starch Pasting Properties.

Starch paste viscosity parameters were determined using a Rapid Visco Analyzer (RVA) essentially as described in Regina et al., (2004). The temperature profile for the RVA comprised the following stages: hold at 60° C. for 2 min, heat to 95° C. over 6 min, hold at 95° C. for 4 min, cool to 50° C. over 4 min, and hold at 50° C. for 4 min. The results showed that the peak and final viscosities were significantly lower in starch from the triple-null ssIIa grain (105.5 and 208.6, respectively) compared to the wild-type wheat starch (232.3 and 350.6, respectively).

Starch Gelatinisation Properties.

Gelatinisation properties of the starch were studied using differential scanning calorimetry (DSC) as described in Regina et al., (2004). DSC was carried out on a Perkin Elmer Pyris 1 differential scanning calorimeter. Starch and water were premixed at a ratio of 1:2 and approximately 50 mg weighed into a DSC pan which was sealed and left to equilibrate overnight. A heating rate of 10° C. per minute was used to heat the test and reference samples from 30 to 130° C. Data was analysed using the software available with the instrument. The results clearly showed a reduced end gelatinisation temperature (61.5° C.) for starch from the triple-null ssIIa grain compared to the control (67.1° C.). The peak gelatinisation temperature was also lower in the triple-null ssIIa starch (55.4° C.) compared to the control starch (61.3° C.). Therefore, starch from the triple-null ssIIa grain has significantly lower gelatinisation temperature and reduced enthalpy of gelatinisation.

Grain Compositional Changes.

Proximate composition of triple-null ssIIa wheat grain was analysed to determine the changes induced in grain components by loss of SSIIa activity. The sucrose level in the ssIIa wholemeal flour was increased relative to that from a wild-type wheat, NB1. The overall sugar content was also higher in the ssIIa mutant starch compared to wild-type starches. Wholemeal from the ssIIa grain and a second grain comprising an hp-SBEIIa genetic construct (Regina et al., 2006) had an increased level of protein of >19% in the grain while wild-type wholemeal had a level within the range of 11-13%. The total dietary fibre (TDF) was higher in the ssIIa starch with a level of 19.2% compared to the wild-type value of 11.0%. The level of other grain components such as neutral non-starch polysaccharides (NNSP), total anti-oxidants and total lipids was comparatively higher in the ssIIa flour compared to that of the wild-type wheat. For NNSP, the highest value of 13.8% was recorded for the ssIIa mutant compared to 8.3% in the wild-type NB1. Total starch content was about 53% in the ssIIa mutant grain compared to >60% in NB1 and YDH7.

### Example 8: Production of Breads and Other Food Products

Food products such as bread and breakfast cereals are effective ways of delivering the modified wheat starch into the diet. To show that the high amylose wheat could readily be incorporated into breads and breakfast cereals and to examine the factors that allowed retention of the food quality, samples of flour were produced, analysed and used in baking or extrusion experiments. Small-scale bread bakes were carried out using YDH7 flour with three levels of addition, 30%, 60% and 100% in comparison with the bakes from flours from grain comprising the hp-SBEIIa genetic construct and the wild-type NB1. Increasing the addition level of either YDH7 or hp-SBEIIa flour resulted in significant increases in RS and reductions in GI.

An extruded breakfast cereal was prepared from wholemeal from the ssIIa mutant wheat and compared to a corresponding breakfast cereal from wild-type wheat. The breakfast cereal contained an increased level of RS (4.3%) when using the triple-null ssIIa wheat compared to the wild-type wheat (1.3%). Increasing the melt temperature from 110° C. to 140° C. in the extruded process slightly reduced the RS content when using the triple-null ssIIa wheat. The results also showed that the wholemeal breakfast cereals from the triple-null ssIIa wheat had a lower HI (hydrolysis index for estimating GI value) value of 72 compared to that from the wild-type wheat at 82.

The following methods are employed for the production of bread from ssIIa wheat at a larger scale. Quantities of wheat grain are conditioned to 16.5% moisture content overnight before milling and sieving to achieve a final average particle size of about 150 μm. The protein and moisture content of the milled samples are determined by infrared reflectance (NIR) according to AACC Method 39-11 (1999), or by the Dumas method using an air-oven according to AACC Method 44-15 A (AACCs 1999).

Micro Z-Arm Mixing.

Optimum water absorption values of wheat flours are determined with a Z-arm Mixer, for example using 4 g of test flour per mix (Gras et al., (2001); Bekes et al., (2002) with constant angular velocity with shaft speeds for the fast and slow blades of 96 and 64 rpm, respectively. Mixing is carried out for about 20 minutes. Before adding water to the flour, the baseline is automatically recorded for 30 sec by mixing only the solid components. The water addition is carried out in one step using an automatic water pump. The following parameters are determined from the individual mixing experiments by taking the averages: WA %—Water Absorption is determined at 500 Brabender Unit (BU) dough consistency; Dough Development Time (DDT): time to peak resistance (sec).

Mixograms.

To determine optimal dough mixing parameters with the modified wheat flour, samples with variable water absorption corresponding to water absorption determined by the Z-arm mixer, are mixed in a Mixograph keeping the total dough mass constant. For each of the flour samples, the following parameters are recorded: MT—mixing time (sec); PR—Mixograph peak resistance (Arbitrary Units, AU); BWPR—band width at peak resistance (Arbitrary Units, AU); RBD—resistance breakdown (%); BWBD—bandwidth breakdown (%); TMBW—time to maximum bandwidth (s); and MBW—maximum bandwidth (Arbitrary Units, A.U.). Dough extensibility parameters are measured as follows: doughs are mixed to peak dough development in a Mixograph. Extension tests at 1 cm/sec are carried out on a TA.XT2i texture analyser with a modified geometry Kieffer dough and gluten extensibility rig (Mann et al., 2003). Dough samples for extension testing (˜1.0 g/test) are moulded with a Kieffer moulder and rested at 30° C. and 90% RH for 45 min. before extension testing: The R_Max and Ext_Rmax are determined from the data with the help of Exceed Expert software (Smewing, TX2 texture analyzer handbook, SMS Ltd: Surrey, UK, 1995).

An illustrative recipe based on 14 g of flour as 100% is as follows: flour 100%, salt 2%, dry yeast 1.5%, vegetable oil 2%, and improver (ascorbic acid 100 ppm, fungal amylose 15 ppm, xylanase 40 ppm, soy flour 0.3%, obtained from Goodman Fielder Pty Ltd, Australia) 1.5%. The water addition level is based on the Z-arm water absorption values that are adjusted for the full formula. Flour (14 g) and the other ingredients are mixed to peak dough development time in a Mixograph. The moulding and panning is carried out with two proofing steps at 40 C at 85% RH. Baking is carried out in a Rotel oven for 15 min at 190° C. Loaf volume (determined by the canola seed displacement method) and weight measurements are taken after cooling the loaves on a rack for 2 hours. Net water loss is measured by weighing the loaves over time.

The flour or wholemeal may be blended with flour or wholemeal from non-modified wheats or other cereals such as barley to provide desired dough and bread-making or nutritional qualities. For example, flour from cvs Chara or Glenlea has a high dough strength while that from cv Janz has a medium dough strength. In particular, the levels of high and low molecular weight glutenin subunits in the flour is positively correlated with dough strength, and further influenced by the nature of the alleles present.

Flour from line YDH7 was used at 100%, 60% and 30% addition levels according to the methods described above for dough preparation and bread making. That is, either 100% of the flour came from the YDH7 or control flour, or 60% or 30% by weight of YDH7 flour was blended with the Baking Control (B. extra) flour. Percentages were of total flour in the bread formulation. The unmodified wheat flour was from wild-type cv. NB1. The grain samples were milled in a Brabender Quadramat Junior mill. All flour blends had their water absorption determined on a Z-arm mixer and optimal mixing times determined on Mixograph as described above. These conditions were used in preparing the test bake loaves.

Mixing Properties.

Apart from the control sample using entirely wild-type flour (Baking Control, NB1), all other flour samples gave elevated water absorption values. Increased incorporation levels of flour from the YDH7 line also led to a decrease in the optimal Mixograph mixing times. In keeping with the water absorption data, the breads including flour from the YDH7 line all showed a reduction in specific loaf volume (loaf volume/loaf weight), correlating with increasing levels of addition of the YDH7 flour.

These studies showed that breads with commercial potential, including acceptable crumb structure, texture and appearance, could be obtained using the modified ssIIa wheat flour blended with control flour samples. Furthermore, high amylose ssIIa wheats may be used in combination with preferred genetic background characteristics (e.g. preferred high and low molecular weight glutenins), or modifications in the food processing such as, for example, the addition of improvers such as gluten, ascorbate or emulsifiers, or the use of differing bread-making styles (e.g. sponge and dough bread-making, sour dough, mixed grain, or wholemeal) to provide a range of products with particular utility and nutritional efficacy for improved bowel and metabolic health.

Other Food Products:

Yellow alkaline noodles (YAN) (100% flour, 32% water, 1% Na₂CO₃) are prepared in a Hobart mixer using the standard BRI Research Noodle Manufacturing Method (AFL 029). Noodle sheet is formed in the stainless steel rollers of an Otake noodle machine. After resting (30 min) the noodle sheet is reduced and cut into strands. The dimensions of the noodles are 1.5×1.5 mm.

Instant noodles (100% flour, 32% water, 1% NaCl and 0.2% Na₂CO₃) are prepared in a Hobart mixer using the standard BRI Research Noodle Manufacturing method (AFL 028). Noodle sheet is formed in the stainless steel rollers of an Otake noodle machine. After resting (5 min) the noodle sheet is reduced and cut into strands. The dimensions of the noodles are 1.0×1.5×25 mm. The noodle strands are steamed for 3.5 min and then fried in oil at 150° C. for 45 sec.

Sponge and Dough (S&D) bread. The BRI Research sponge and dough baking involves a two-step process. In the first step, the sponge is made by mixing part of the total flour with water, yeast and yeast food. The sponge is allowed to ferment for 4 h. In the second step, the sponge is incorporated with the rest of the flour, water and other ingredients to make dough. The sponge stage of the process is made with 200 g of flour and is given 4 h fermentation. The dough is prepared by mixing the remaining 100 g of flour and other ingredients with the fermented sponge.

Pasta—Spaghetti. The method used for pasta production is as described in Sissons et al., (2007). Flours from ssIIa modified wheat and wild-type wheat are mixed with Manildra semolina at various percentages (test sample: 0, 20, 40, 60, 80, 100%) to obtain flour mixes for small scale pasta preparation. The samples are corrected to 30% moisture. The desired amount of water is added to the samples and mixed briefly before being transferred into a 50 g farinograph bowl for a further 2 min mix. The resulting dough, which resembles coffee-bean-size crumbs, is transferred into a stainless steel chamber and rested under a pressure of 7000 kPa for 9 min at 50° C. The pasta is then extruded at a constant rate and cut into lengths of approximately 48 cm. The pasta is dried using a temperature and humidity cabinet. The drying cycle uses a holding temperature of 25° C. followed by an increase to 65° C. for 45 min then a period of about 13 h at 50° C. followed by cooling. Humidity is controlled during the cycle. Dried pasta is cut into 7 cm strands for subsequent tests.

### Example 9: In Vitro Measurements of Glycaemic Index (GI) of Food Samples

The Glycemic Index (GI) of food samples was measured in vitro as follows. The in vitro method simulates what happens to food samples when consumed by human subjects and is predictive of in vivo GI measurement. Food samples were homogenised with a domestic food processor. An amount of sample representing approximately 50 mg of carbohydrate was weighed into a 120 ml plastic sample container and 100 μl of carbonate buffer added without α-amylase. Approximately 15-20 seconds after the addition of carbonate buffer, 5 ml of Pepsin solution (65 mg of pepsin (Sigma) dissolved in 65 ml of HCl 0.02 M, pH 2.0, made up on the day of use) was added, and the mixture incubated at 37° C. for 30 minutes in a reciprocating water bath at 70 rpm. Following incubation, the sample was neutralised with 5 ml of NaOH (0.02 M) and 25 ml of acetate buffer 0.2 M, pH 6 added. 5 ml of enzyme mixture containing 2 mg/mL of pancreatin (α-amylase, Sigma) and 28 U/mL of amyloglucosidase from Aspergillus niger (AMG, Sigma) dissolved in Na acetate buffer (sodium acetate buffer, 0.2 M, pH 6.0, containing 0.20 M calcium chloride and 0.49 mM magnesium chloride) was then added, and the mixture incubated for 2-5 minutes. 1 ml of solution was transferred from each flask into a 1.5 ml tube and centrifuged at 3000 rpm for 10 minutes. The supernatant was transferred to a new tube and stored in a freezer. The remainder of each sample was covered with aluminium foil and the containers incubated at 37° C. for 5 hours in a water bath. A further 1 ml of solution was then collected from each flask, centrifuged and the supernatant transferred as before. Supernatants were stored in a freezer until the absorbances could be read.

All supernatants were thawed and centrifuged at 3000 rpm for 10 min. Samples were diluted as necessary (1 in 10 dilution usually sufficient), 10 μl of supernatant transferred from each sample to 96-well microtitre plates in duplicate or triplicate. A standard curve for each microtitre plate was prepared using glucose (0 mg, 0.0625 mg, 0.125 mg, 0.25 mg, 0.5 mg and 1.0 mg). 20 μl of Glucose Trinder reagent (Microgenetics Diagnostics Pty Ltd, Lidcombe, NSW) was added to each well and the plates incubated at room temperature for approximately 20 minutes. The absorbance of each sample was measured at 505 nm using a plate reader and the amount of glucose calculated with reference to the standard curve.

Bread loaves baked using flour from the YDH7 wheat line were tested for GI using the method described above along with bread made from the non-transformed wild-type wheat, as well as blends of the two flours using incorporation levels of 60% and 30% YDH7 flour, the remainder 40% or 70% flour being from the wild-type grain. Increased incorporation of YDH7 flour resulted in significant reductions in GI as measured by the in vitro test.

### Example 10: Processing of High Amylose Wheat and Resultant RS Levels

A small scale study was conducted to determine the resistant starch (RS) content in processed grain from the YDH7 wheat which had been rolled or flaked. The technique involved conditioning the grains to a moisture level of 25% for one hour, followed by steaming the grains. Following steaming, the grains were flaked using a small-scale roller. The flakes were then roasted in an oven at 120° C. for 35 min. Two roller widths and three steaming timings were used on approximately 200 g of samples from high amylose wheat having reduced SSIIa and wild-type, control wheat (cv. Hartog). The roller widths tested were 0.05 mm and 0.15 mm. The steaming timings tested were 60′, 45′ and 35′.

This study showed a clear and substantial increase in the amount of RS in processed high amylose wheat compared to the control. There also appeared to be some effect of the processing conditions on the RS level. For example with the high amylose grain, increased steaming times led to a slight reduction in the level of RS, most likely due to increased starch gelatinization during steaming. The wider roller gap generated a higher RS level except at the longest steaming time. This could have been due to increased shear damage of the starch granules when the grains were rolled at narrower gaps, reducing RS levels slightly. Narrower roller gaps also led to higher RS levels in the Hartog control, albeit at much lower overall RS levels. In contrast to the high amylose results, increased steaming times led to higher RS levels, possibly due to increased starch gelatinization at longer steaming times contributing to more starch retrogradation during subsequent processing and cooling.

### Example 11: Generation and Identification of Additional ssIIa Mutations

Mutagenesis of Wheat by Heavy Ion Bombardment.

Mutagenesis by radiation such as by heavy ion bombardment (HIB) readily produces deletion mutations at practical frequencies in the wheat genome. A mutagenised wheat population was generated in the wheat variety Chara, a commonly used commercial variety, by HIB of wheat seeds. Two sources of heavy ions were used, namely carbon and neon, for mutagenesis which was conducted at Riken Nishina Centre, Wako, Saitama, Japan. Mutagenised seeds were sown in the greenhouse to obtain the M1 plants. These were then selfed to produce the M2 generation. DNA samples were isolated from each of approximately 15,000 M2 plants, each from a different M1 plant.

Each DNA is individually screened for mutations in each of the SSIIa genes by PCR using genome specific oligonucleotide primers which produce amplified fragments for each of the SSIIa genes on the A, B and D genomes. Such diagnostic PCR primers can readily be designed by those skilled in the art by comparing the three genomic nucleotide sequences (SEQ ID NOs:7, 8 and 9) and choosing oligonucleotide sequences which anneal specifically to one genomic sequence but not the other two, or where the resultant amplified fragments are cleaved differentially by restriction enzyme digestion to produce different sized fragments for the three genomic SSIIa genes. Each of the PCR reactions on wild-type (non-mutagenised) DNA samples thereby yield 3 distinct amplification products which corresponded to the amplified regions of SSIIa genes on the A, B and D genomes, whereas the absence of one of the fragments in the PCRs from mutagenized M2 DNA samples indicates the absence of the corresponding region in one of the genomes, i.e. the presence of a mutant allele in which at least part of the gene was deleted. Such mutant alleles would certainly be null alleles. When screened in such a manner using SBEIIa- and SBEIIb-gene specific primers, the 15,000 M2 plants identified a total of 34 mutants which were deletion mutants for the SBEIIa and/or SBEIIb genes (WO2012/058730), indicating that the frequency of deletion mutations for a gene of interest in this mutagenized M2 populations was about 1 per 1000 lines. Screening of the M2 lines therefore identifies about 15 mutants each having a deletion mutation of, or in, an SSIIa gene. Since the SSIIa genes on the A, B and D genomes are distinguished by the diagnostic PCR reactions, the mutant alleles are assigned to one of the genomes according to which amplification product was absent. About 5 mutants are identified for each of the SSIIa-A, SSIIa-B and SSIIa-D genes in this manner from the population of 15,000 M2 plants.

The extent of the chromosome deletion in each of the mutants is determined by microsatellite mapping. Microsatellite markers previously mapped to the short arm of chromosomes 7A, 7B and 7D, the chromosomal locations of the SSIIa genes, are tested on these mutants to determine the presence or absence of each marker in each mutant. Mutant plants in which either all or most of the specific chromosome microsatellite markers were retained, based on the production of the appropriate amplification product in the reactions, are inferred to be relatively small deletion mutants. Such mutants were preferred, considering that it was less likely that other, important genes were affected by the mutations.

Crossing of Mutants.

Mutant plants that were homozygous for smaller deletions of or in an SSIIa gene as judged by the microsatellite marker analysis are selected for crossing to generate progeny plants and grain which have mutant ssIIa alleles on multiple genomes. F1 progeny plants from the crosses are selfed, and F2 seed obtained and analysed for their SSIIa genotype. Such mutants can also be crossed with mutants comprising point mutations in SSIIa genes to produce triple gene mutants having combinations of deletions and point ssIIa mutations.

Point Mutations.

Point mutations including single nucleotide polymorphisms (SNP) can be identified in publically available libraries of mutagenized wheat plants. Such libraries include, for example, one available from John Innes Centre, UK. A wheat SSIIa nucleotide sequence (Genbank Accession No: AB201445) was used to interrogate the John Innes Centre wheat database using BLAST software and lines comprising SNPs in each of the three SSIIa genes were identified. The SNPs were categorized into three classes. The first group comprised mutants which had a mutated SSIIa gene comprising a new stop codon in the protein coding region of the gene. These mutations were predicted to cause premature termination of translation of the SSIIa protein encoded by that gene. Premature termination mutations, also known as nonsense mutations or “stop codon-gained mutations”, are almost always null mutations provided the mutation is not close to the 3′ end of the protein coding region, although even those may be null mutations. The second group of mutants comprised lines which had a nucleotide polymorphism in a splice site of an SSIIa gene, in either a splice donor site or a splice acceptor site. Such mutations were expected to cause mis-splicing of the RNA transcript from the SSIIa gene and severely affect the mRNA; splice-site mutations are most often null mutations. The third group consisted of mutants which comprised a point mutation in one of the SSIIa genes which resulted in an amino acid substitution in the encoded SSIIa polypeptide; these are termed “missense mutations”. The impact of each missense mutation on the structure of the encoded protein is predicted using Blosum 62 and Pam 250 matrices.

The SSIIa gene mutants which were identified in the first and second groups are listed in Table 9. For the SSIIa-A gene, 5 nonsense mutations, 2 splice-site mutations and 49 missense mutations were identified from the database. Two of the nonsense mutations, identified in different pools, were identical. For the SSIIa-B gene, 2 nonsense mutations, 7 splice site variants and 22 missense mutations, were identified. For the SSIIa-D gene, 2 splice mutations and 49 missense mutations were identified. Several mutants had more than one polymorphism in an SSIIa gene, including some which had a polymorphism in an intron as well as a polymorphism in a protein coding region.

Identification of Mutations by TILLING.

A population of mutated plant lines was developed after sodium azide mutagenesis of seeds of the wheat cultivar Sunstate, using the method described in WO2014/028980. Five thousand M1 plants were grown to maturity, allowing them to self-fertilise. M2 seeds were harvested separately from each plant, thereby producing the mutagenized population of 5000 lines. DNA was extracted from a leaf section (˜2 cm) from plants from each of the lines. Wheat leaf DNAs were quantified using a plate reader (FLUOstar Omega, BMG LABTECH) and normalized to 10 ng/μl using a robot machine (Corbett Life Science). For TILLING, DNA samples from sets of 8 plants were pooled together in each 96 plate well. PCR reactions were conducted to amplify segments of the SSIIa genes, for example with one cycle of 95° C. for 5 min, then 35 cycles of melting at 94° C. for 45 s, annealing temperature of 60 to 62° C. for 30 s, and extension at 72° C. for 2 min 30 s, then 1 cycle of 72° C. for 10 min, followed by cooling to 25° C. The resultant PCR fragments (5 μl) were separated on 1 or 2% agarose gels and visualized (UVitec) after ethidium staining for examining the quality of PCR amplification.

Heteroduplexing of PCR fragments to the wild-type RNA transcript is carried out using a PCR machine with 1 cycle of 99° C. for 5 min, 70 cycles of annealing starting at 70° C. and reducing at a rate of 0.6° C. per cycle, for 20 s, followed by cooling to 25° C. The heteroduplexes are digested with the Cell enzyme, using 5 μl of heteroduplexed PCR product with 5 μl of 2× buffer mixture and 0.5 μl of Cell enzyme. The buffer mixture is composed of 20 mM HEPES, pH 7.5, 20 mM MgSO₄, 20 mM KCl, 0.004% Triton X-100 and 0.4 μg/ml BSA. The assembled reactions are mixed and incubated at 45° C. for 15-30 min. The digested DNA samples are then loaded onto a DNA fragment analyser (Advanced Analytical Technologies) for separating DNA fragments according to the Mutation Discovery kit (DNF-910-K1000T). Alternatively, sets of 10 amplification products are pooled after normalization of the PCR products, and sequenced with one DNA pool per flow cell. The sequence data is analysed to select lines having ssIIa gene polymorphisms. SNP assays are designed for each polymorphism based on kaspar technology, and genotyping is performed on the M1 lines in each pool that is positive for a particular polymorphism. Thereby, the individual mutant line containing each mutant gene is identified and the mutant SSIIa sequences confirmed.

Plants containing a point mutation in a single SSIIa gene are crossed with plants containing mutations in the other two SSIIa genes to generate triple-null ssIIa mutants.

TABLE 9
ssIIa mutants identified in the JIC mutagenized library

| | | | | | | | | | |
|---|---|---|---|---|---|---|---|---|---|
| | | | | | | | | Amino | |
| | | | Mutation | Chromosome | WT | Mutant | cDNA | acid | Codon |
| Line | Chromosome | Mutation type | Position | position | base | base | position | change | change |

ssIIa-A mutants

| | | | | | | | | | |
|---|---|---|---|---|---|---|---|---|---|
| Kronos2261 | 7AS | Stop codon gained | 322 | 52346758 | C | T | 1013 | W269* | tgG/tgA |
| Cadenza0110 | 7AS | Stop codon gained | 114 | 52346550 | G | A | 1221 | Q339* | Cag/Tag |
| Cadenza0403 | 7AS | Stop codon gained | 114 | 52346550 | G | A | 1221 | Q339* | Cag/Tag |
| Cadenza1738 | 7AS | Stop codon gained | 3460 | 52349896 | C | T | 563 | W119* | tgG/tgA |
| Cadenza1097 | 7AS | Stop codon gained | 323 | 52346759 | C | T | 1012 | W269* | tGg/tAg |
| Cadenza1237 | 7AS | Splice acceptor site | 5153 | 52351589 | C | T | N/A | N/A | N/A |
| | | variant | | | | | | | |
| Cadenza0413 | 7AS | Splice region variant | 5081 | 52351517 | G | A | N/A | N/A | N/A |
| | | & intron variant | | | | | | | |

ssIIa-B mutants

| | | | | | | | | | |
|---|---|---|---|---|---|---|---|---|---|
| Cadenza1731 | 7BS | Stop codon gained | 1811 | 31821582 | G | A | 342 | W82* | tGg/tAg |
| Kronos3900 | 7BS | Stop codon gained | 1812 | 31821583 | G | A | 343 | W82* | tgG/tgA |
| Kronos2209 | 7BS | Splice acceptor variant | 1172 | 31820943 | G | A | N/A | N/A | N/A |
| Cadenza1031 | 7BS | Splice region variant | 40 | 31819811 | G | A | N/A | N/A | N/A |
| | | & intron variant | | | | | | | |
| Cadenza1788 | 7BS | Splice region variant | 41 | 31819812 | G | A | N/A | N/A | N/A |
| | | & intron variant | | | | | | | |
| Kronos2375 | 7BS | Splice region variant, | 1001 | 31820772 | C | T | N/A | N/A | N/A |
| | | intron variant | | | | | | | |
| Kronos2187 | 7BS | Splice region variant, | 1088 | 31820859 | G | A | N/A | N/A | N/A |
| | | intron variant | | | | | | | |
| Kronos3229 | 7BS | Splice region variant, | 1167 | 31820938 | C | T | N/A | N/A | N/A |
| | | intron variant | | | | | | | |
| Cadenza1176 | 7BS | Splice region variant | 1378 | 31821149 | C | T | N/A | N/A | N/A |
| | | & intron variant | | | | | | | |

ssIIa-D mutants

| | | | | | | |
|---|---|---|---|---|---|---|
| Cadenza0627 | 7DS | Splice region variant, | 555 | N/A | N/A | N/A |
| | | intron variant | | | | |
| Cadenza1762 | 7DS | Splice region variant, | 1612 | N/A | N/A | N/A |
| | | intron variant | | | | |

### Example 12: Analysis of Proteins in ssIIa Mutant Grain

Expression of Starch Biosynthetic Genes in ssIIa Mutants.

Five ssIIa mutant wheat lines, designated A24, B22, B29, B63 and E24, were selected from the double haploid population of Konik-Rose et al. (2007) and the content of several starch biosynthetic mRNAs and proteins analysed as follows. The level of mRNA expression in developing grains at 15 DPA of the ssIIa mutant wheat plants was compared to the wild-type by quantitative reverse transcription PCR (qRT-PCR) as described in Example 1. Quantitation of RNA in each sample was done by reference to a constitutively-expressed tubulin gene. The qRT-PCR data revealed that the level of ssIIa mRNA in the homozygous triple mutant ssIIa endosperm was significantly lower than that in the corresponding wild-type grains (p<0.01), being only about 8% of the level relative to the corresponding wild-type wheat endosperm. In contrast, the levels of SSI, SBEIIa and SBEIIb transcripts in the mutant grain was about the same as in the corresponding wild-type developing grain. This indicated a specific effect on ssIIa mRNA in the mutant endosperm.

Analyses of the Abundance of Granule Bound Proteins in the Starch of Mature Grain.

To compare protein band intensity on a protein gel and the amount of protein sample loaded on the gel, one to four mg of starch as starch granules from mature ssIIa mutant and SSIIa wild-type grains were used for the extraction of starch granule bound proteins. The proteins were separated on a protein gel as described in Example 1. Four proteins with molecular weight of at least 60 kDa were observed to be bound to the starch granules in the wild-type mature grain. In the ssIIa mutant grain, a single protein, identified as GBSSI, was observed at approximately 60 kDa. When the protein bands were quantitated, there was a positive correlation between the protein band intensity and the amount of starch used for protein extraction for each of SSIIa, SBEII and SSI from the wild-type grain and for GBSSI in the mutant grain. Due to the low levels of SSIIa, SBEII and SSI proteins inside the starch granules, an amount of four mg starch was selected for extracting starch granule bound proteins for the further analysis described as follows.

Four starch granule bound proteins having a molecular weight of about 60 kDa or greater were observed when the protein extracts of wild-type wheat grain were subjected to gel electrophoresis and stained with Sypro. They were identified as SSIIa (˜88 kDa), SBEIIa and SBEIIb (each ˜83 kDa), SSI (˜75 kDa) and GBSSI (˜60 kDa) polypeptides by immunonblot analyses. SBEIIa and SBEIIb migrated at almost the same position on the gels, but the abundance of SBEIIb inside the wild-type starch granules was much greater than SBEIIa as judged by the Sypro staining.

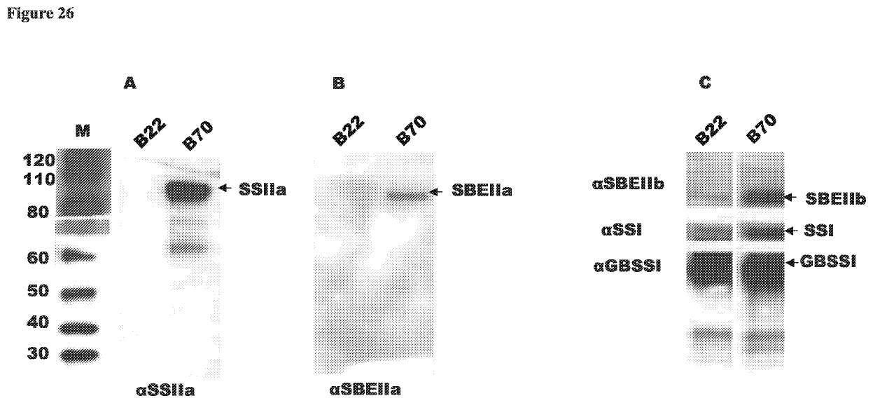

When the proteins from the ssIIa mutant wheat starch granules were analysed by gel electrophoresis, SSIIa and SBEIIa proteins were not detected. SSI and SBEIIb proteins were not detected by Sypro staining but could be detected by immunoblot analyses although their abundance was so low that accurate quantitation could not be made (FIG. 26).

Analysis of the Protein Abundance of Starch Biosynthetic Enzymes in the Soluble Stroma and Inside the Starch Granules of Developing Endosperm.

To determine whether the low concentration of SSI, SBEIIa and SBEIIb inside the starch granules in the mature, mutant grain was due to a lower rate of synthesis during grain development, at the time when SSIIa protein was absent in the mutant grain, the SSI, SBEIIa and SBEIIb proteins in the soluble stroma fraction and inside the starch granules of the developing endosperms were quantitated at 15 DPA. Quantitation was done by immunodetection. When the soluble proteins of the developing endosperms were analysed, significantly increased amounts of both SBEIIb and SSI were present in the ssIIa mutant grain compared to the SSIIa wild-type developing grain at the same stage (15 DPA). In contrast, the level of SBEIIa was similar in the ssIIa mutant and SSIIa wild-type grain. SBEIIa was more abundant in the soluble fraction of the amyloplasts than SBEIIb. It was concluded that the reduced levels of SSI, SBEIIa and SBEIIb proteins inside the starch granules was not due to reduced expression levels but instead reflected a reduced binding in the starch granules, indicating a localisation away from the starch granules and into the soluble fraction in the stroma.

When the starch granule bound proteins in the developing grain were analysed, no SSIIa protein was detected in the mutant wheat ssIIa grain by immunoblot analyses. The level of SBEIIb was decreased to about 25% compared to the wild-type. The level of SSI inside the starch granules of the mutant ssIIa was also approximately 25% of the wild-type level. Similar patterns of reduction in the amount of starch granule bound proteins were observed in the mature endosperms. However, on a per starch basis, the concentration of starch biosynthetic enzymes in starch granule bound proteins of developing endosperms was higher than that in mature grains as a result of the dilution effect of higher starch levels in mature grain endosperm.

### BIBLIOGRAPHY

Abel et al., (1996). Plant Journal 10:981-991.
Altschul et al., (1997). Nucl. Acids Res. 25:3389-3402.
Alvarez et al., (2006). Plant Cell 18:1134-1151.
Asai et al., (2014). J. Exper. Biol. 65:5497-5507.
Batey and Curtin, (1996). Starch 48:338-344.
Batey et al., (1997). Cereal Chemistry 74:497-501.
Bechtold et al., (1993). C.R. Acad. Sci. Paris, 316:1194-1199.
Bekes et al., (2002). Crit Care Med. 30:1906-1907.
Birt et al., (2013). Adv. Nutrition 4:587-601.
Boch (2011). Nature Biotechnology, 29:135-136.
Botticella et al., (2011). BMC Plant Biology 11: 156.
Boyer and Preiss, (1978). Carbohydrate Research, 61: 321-334.
Boyer and Preiss, (1981). Plant Phys. 67: 1141-1145.
Bryan et al., (1998). J. Cereal Sci. 28:135-145.
Butardo et al., (2012). J Agr Food Chem 60:11576-11585. Ausubel et al., “Current Protocols in Molecular Biology”, John Wiley & Sons Inc, Chapter 15, 1994-1998
Carpita et al., (1993). Plant J. 3:1-30.
Carpita et al., (2011) Plant Physiol. 155:171-184.
Case et al., (1998). Journal of Cereal Science 27: 301-314.
Castro et al., (2005). Biomacromolecules 6: 2260-2270.
Cheng et al., (1997). Plant Physiol 115: 971-980.
Colgrave et al., (2013). Food Res Int 54:1001-1012.
Comai et al., (2004). Plant J. 37: 778-786.
Doudna et al., (2014) Science 346 (6213).
Dupuis et al., (2014). Comprehensive Reviews in Food Science and Food Safety 13:1219-1234.
Durai et al., (2005). Nucleic Acids Research 33: 5978-5990.
Eagles et al., (2001). Aust. J. Agric. Res. 52: 1 349-1356.
Ellison et al., (1994) Aust. J. Exp. Agric. 34:869-869.
Fergason, (1994). Speciality Corns eds, CRC Press Inc. pp 55-77.
Fontaine et al., (1993). J Biol Chem 268:16223-16230.
Fujita et al., (2006). Plant Physiol 140:1070-1084.
Fuwa et al., (1999). Starch-Starke 51: 147-151.
Gao et al., (1997). Plant Physiol 114: 69-78.
Gao and Chibbar (2000). Genome 43:768-775.
Giddings et al., (1980). J Cell Biol. 84:327-339.
Gras et al., (2001). In: Proc. ICC 11th Int. Cereal and Bread Congress. RACI: Melbourne.
Gruppen et al., (1992). J. Cereal Sci. 16:41-51.
Guan and Keeling (1998). Trends Glycosci. Glycotechnol. 10:307-319.
Earn et al., (1998). Plant Mol. Biol. 37:639-649.
Hartmann and Endres, (1999). Manual of Antisense Methodology, Kluwer.
Hayashi et al., (2007). Cyclotrons and Their Applications. Eighteenth International Conference 237-239.
Hedman and Boyer, (1982). Biochemical Genetics 20: 483-492.
Henikoff et al., (2004). Plant Physiol. 135: 630-636.
Henry (1985). J. Sci. Food Agric. 36:1243-1253.
Hinchee et al., (1988). Biotech., 6: 915-922.
Hopkins et al., (2003). Appl. Environ. Microbiol. 69:6354-6360.
Hung et al., (2006). Trends Food Sci. Technol. 17:448-456.
Huynh et al (2008a). J Cereal Sci. 48:369-378.
Huynh et al (2008b). Theor. Appl. Genet. 117:701-709.
Jobling et al., (1999). Plant Journal 18: 163-171.
Jolly et al. (1996). Proc. Natl. Acad. Sci. U.S.A. 93: 2408-2413.
Juong et al, (2013). Nat Rev Mol Cell Biol 14:49-55.
Kanna and Daggard, (2003). Plant Cell Rep 21: 429-436.
Karppinen et al., (2003). Cereal Chem. 80:168-171.
Kaur and Gupta (2002) J. Biosci. 27:703-714.
Kawaura et al., (2009). BMC Genomics 10:271.
Kazama et al., (2008). Plant Biotechnology 25: 113-117.
Klein et al., (1987). Nature 327: 70-73.
Konik-Rose et al., (2001). Starch-Starke, 53: 14-20.
Konik-Rose et al., (2007). Theor Appl Genet 115:1053-1065.
Kosar-Hashemi et al., (2007). Funct Plant Biol. 34:431-438.
Krueger et al., (1987). Cereal Chemistry 64: 187-190.
Langridge et al., (2001). Aust. J. Agric. Res. 52: 1043-1077.
Lazaridou et al., (2007). In: Biliaderis and Izydorczyk (Eds.) Functional food carbohydrates (pp 1-72). CRC Press, Boca Raton Fla.
Lazo et al., (1991). Biotechnology (N Y). 9(10): 963-967.
Le Provost et al., (2009). Trends in Biotechnology 28(3): 134-141.
Leterrier et al., (2008) BMC Plant Biol. 8:98.
Li et al., (1999a). Plant Physiol 120:1147-1155.
Li et al., (1999b). Theor. Appl. Genet. 98: 1208-1216.
Li et al., (2000). Plant Physiol 123: 613-624.
Li et al., (2003). Funct. Integr. Genomics 3:76-85.
Liu et al., (2010). Biotechnology and Bioengineering, 106: 97-105.
Luo et al., (2015). Theor. Appl. Genet. 128: 1407-1419.
Mann et al., (2003). In ‘Workshop on gluten proteins’ Eds. Lafiandra et al., The Royal Society of Chemistry: Cambridge, UK, pp. 215-218.
Matsumoto et al., (2011). Plant Physiol. 156:20-28.
McCleary et al., (2000). J AOAC Int. 83:997-1005.
McCleary et al., (2013). Cereal Chem. 90:396-414.
Mizuno et al., (1992). J. Biochem 112:643-651.
Mizuno et al., (1993). J. Biol. Chem. 268: 19084-19091.
Morell et al., (1998). Electrophoresis 19: 2603-2611.
Morell et al., (1997). Plant Physiol 113: 201-208.
Morell et al., (2003). Plant J 34:172-184.
Morrison and Laignelet, (1983). J Cereal Sci, 1: 9-20.
Morrison et al., (1984). J Cereal Sci. 2:257-271.
Nakamura et al., (1996). Planta 199:209-218.
Nakamura and Yamanouchi, (1992). Plant Physiol. 99:1265-1266.
Needleman and Wunsch, (1970). J. Mol. Biol. 48:443-453.
Niedz et al., (1995). Plant Cell Reports, 14:403-406.
Nishi et al., (2001). Plant Physiol 127:459-472.
Ohdan et al., (2005). J Exp Bot 56:3229-3244.
O'Shea and Morell (1996). Electrophoresis 17:681-686.
Ow et al., (1986). Science, 234:856-859.
Palatnik et al., (2003). Nature 425:257-263.
Parizotto et al., (2004). Genes Dev 18:2237-2242.
Potrykus et al., (1985). Mol. Gen. Genet., 199:183-188.
Prasher et al., (1985). Biochem. Biophys. Res. Comm. 126:1259-1268.
Prosky et al., (1985). J Assoc. Off. Anal. Chem. Int. 68:677-679.
Quraishi et al., (2011). Funct Integr Genomics 11:71-83.
Rahman et al., (1999). Theor. Appl. Genet. 98:156-163.
Rahman et al., (1995). Aust. J. Plant Physiol. 22:793-803.
Rahman et al., (2001). Plant Physiol 125:1314-1324.
Regina et al., (2006). Proc. Natl. Acad. Sci. U.S.A. 103:3546-3551.
Regina et al., (2005). Planta 222:899-909.
Regina et al., (2010). J Exp Bot. 61(5): 1469-1482.
Regina et al., (2004). Funct Plant Biol, 31: 591-601.
Ritsema and Smeekens (2003) Curr. Opin. Plant Biol. 6:223-230.
Salanoubat et al., (2000). Nature 408:820-822.
Schnable et al., (2009). Science 326:1112-1115.
Schondelmaier et al., (1992) Plant Breeding 109:274-280.
Schulman and Kammiovirta, (1991). Starch 43: 387-389.
Schwab et al., (2006). Plant Cell 18: 1121-1133.
Schwall et al., (2000). Nature Biotechnology 18: 551-554.
Sestili et al., (2010). Mol Breeding 25:145-154.
Sharp et al., (2001). Aust J Agric Res 52: 1357-1366.
Shimbata et al., (2005). Theor. Appl. Genet. 111:1072-1079.
Sidebottom et al., (1998). J. Cereal Sci 27: 279-287.
Sissons et al., (2007). J. Sci. Food and Agric. 87: 1874-1885.
Slade and Knauf, (2005). Transgenic Res. 14: 109-115.
Smewing, (1995). TX2 texture analyzer handbook, SMS Ltd: Surrey, UK.
Smith et al., (2000). Nature, 407: 319-320.
Smith and Hartley (1983). Carbohydrate Research 118: 65-80.
Sparks and Jones (2004). In Transgenic Crops of the World-Essential protocols, Ed. IP Curtis, Kluwer Academic Publishers, Dordrecht, Netherlands, pp 19-34.
Stalker et al., (1988). Science, 242: 419-423.
Steegmans et al (2004). J. AOAC Int. 87, 1200e1207.
Stone and Morell (2009). In: Khan and Shewry (Eds.) Wheat: Chemistry and Technology. American Association of Cereal Chemists, St Paul, Minn., pp 299-362.
Sun et al., (1997). New Phytol. 137:215-222.
Sun et al., (1998). Plant Physiol 118: 37-49.
Sune et al., (2013). Biotechnology and Bioengineering 110:1811-1821.
Takeda et al., (1993). Carbohydrate Research 246: 273-281.
Tanaka et al., (1990). Nucl. Acids Res. 18: 6767-6770.
Tilley (2004). Cereal Chem 81:44-47.
Toyota et al., (2006). Planta 223:248-257.
Tungland and Meyer (2002) Comprehensive Reviews in Food Science and Food Safety 2:73-77.
Waterhouse et al., (1998). Proc. Natl. Acad. Sci. U.S.A. 95: 13959-13964.
Weir et al., (2001). Aust J Plant Physiol 28: 807-818.
Whelan et al (2011). Int J. Food Sci. Nutr. 62:498-503.
Wu et al., (2003). Plant Cell Rep 21: 659-668.
Yamamori et al., (2000). Theor Appl Genet 101:21-29.
Yamamori et al., (2006). Aust. J. Agric. Res. 57:531-535.
Yan et al., (2008). Plant Sci. 176:51-57.
Zwar and Chandler, (1995). Planta 197: 39-48.

1. Wheat grain of the species Triticum aestivum, the grain comprising
i) mutations in each of its SSIIa genes such that the grain is homozygous for a mutation in its SSIIa-A gene, homozygous for a mutation in its SSIIa-B gene and homozygous for a mutation in its SSIIa-D gene, wherein at least two of the mutations in said SSIIa genes are null mutations,
ii) a total starch content comprising an amylose content and an amylopectin content,
iii) a fructan content which is increased relative to wild-type wheat grain on a weight basis, preferably between 3% and 12% of the grain weight,
iv) a β-glucan content,
v) an arabinoxylan content,
vi) a cellulose content,
the grain having a grain weight of between 25 mg and 60 mg, wherein the amylose content is between 45% and 70% on a weight basis of the total starch content of the grain as determined by iodine binding assay, wherein the amylopectin content on a weight basis is reduced relative to the wild-type wheat grain, wherein each of the β-glucan content, arabinoxylan content and cellulose content are increased relative to the wild-type wheat grain on a weight basis, such that the sum of the fructan content, β-glucan content, arabinoxylan content and cellulose content is between 15% and 30 of the grain weight.

2. The wheat grain of claim 1 which is further characterised by one or more or all of the features:
i) a starch content of between 30% and 70% of the grain weight,
ii) the amylose content is between 45% and 65% of the total starch content of the grain as determined by iodine binding assay,
iii) the starch content has a chain length distribution as determined by fluorescence-activated capillary electrophoresis (FACE) after debranching of the starch samples which is increased in the proportion of chain lengths of DP 7-10 and decreased in the proportion of chain lengths DP 11-24, relative to wild-type wheat starch,
iv) the fructan content comprises fructan of DP 3-12 such that at least 50% of the fructan content is of DP 3-12,
v) the fructan content is increased by between 2-fold and 10-fold relative to the wild-type wheat grain on a weight basis,
vi) the β-glucan content is increased by 1% or by 2% on an absolute basis, and/or is increased by between 2-fold and 7-fold relative to the wild-type wheat grain on a weight basis,
vii) the β-glucan content is between 1% and 4% of the grain weight,
viii) the arabinoxylan content is increased by between 1% and 5% on an absolute basis,
ix) the cellulose content is increased by between 1% and 5% on an absolute basis,
x) the grain has a germination rate which is between about 70% and about 100% relative to the wild-type wheat grain,
xi) the grain, when sown, gives rise to wheat planes which are male and female fertile.

3. The grain of claim 1, wherein the grain comprises a level and/or activity of SSIIa protein which is less than 5% of the level or activity of SSIIa protein in the wild-type wheat grain, or which lacks one or more or all of SSIIa-A protein, SSIIa-B protein and SSIIa-D protein.

4. The grain of claim 1, wherein the grain is homozygous for a null mutation in its SSIIa-A gene, homozygous for a null mutation in its SSIIa-B gene and homozygous for a null mutation in its SSIIa-D gene.

5. The grain of claim 1, wherein each null mutation is selected, independently, from the group consisting of a deletion mutation, an insertion mutation, a premature translation stop codon, a splice site mutation and a non-conservative amino acid substitution mutation, preferably wherein the grain comprises deletion mutations in each of two or three SSIIa genes.

6-11. (canceled)

12. The grain of claim 1, wherein the grain comprises starch granules and/or starch characterised by one or more of properties selected from the group consisting of:
(i) comprising at least 2% resistant starch;
(ii) the starch characterised by a reduced glycaemic index (GI);
(iii) the starch granules being distorted in shape;
(iv) the starch granules having reduced birefringence when observed under polarized light;
(v) the starch characterized by a reduced swelling volume;
(vi) modified chain length distribution and/or branching frequency in the starch;
(vii) the starch characterized by a reduced peak temperature of gelatinisation;
(viii) the starch characterized by a reduced peak viscosity;
(ix) reduced starch pasting temperature;
(x) reduced peak molecular weight of amylose as determined by size exclusion chromatography;
(xi) reduced starch crystallinity; and
(xii) reduced proportion of A-type and/or B-type starch, and/or increased proportion of V-type crystalline starch;
each property being relative to wild-type wheat starch granules or wild-type wheat starch.

13-15. (canceled)

16. A wheat plant which produces, or is obtained from, the grain of claim 1.

17. The wheat plant of claim 16 which is characterised by a level and/or activity of SSIIa protein in its endosperm which is less than 5% of the level or activity of SSIIa protein in the wild-type wheat grain, or which lacks one or more or all of SSIIa-A protein, SSIIa-B protein and SSIIa-D protein.

18. (canceled)

19. Flour or wheat bran produced from the grain of claim 1.

20. Wheat starch granules or wheat starch produced from the grain of claim 1.

21. (canceled)

22. A food ingredient comprising flour or wheat bran, or both, at a level of at least 10% on a dry weight basis, wherein the flour or bran was produced from the grain of claim 1.

23. (canceled)

24. A food product comprising a food ingredient at a level of at least 10% on a dry weight basis, wherein the food ingredient is wheat grain of claim 1, or flour or wheat bran produced therefrom.

25. (canceled)

26. A process for producing wheat grain, comprising harvesting wheat grain from a wheat plant which comprises mutations in each of its SSIIa genes such that the grain is homozygous for a mutation in its SSIIa-A gene, homozygous for a mutation in its SSIIa-B gene and homozygous for a mutation in its SSIIa-D gene, wherein at least two of the mutations in said SSIIa genes are null mutations, wherein the harvested wheat grain is grain according to claim 1.

27. A process for producing a wheat plant that produces grain according to any one of claims claim 1 to 13, the process comprising step (i) crossing two parental wheat plants each comprising a null mutation in each of one, two or three SSIIa genes selected from the group consisting of SSIIa-A, SSIIa-B and SSIIa-D, or of mutagenising a parental plant comprising said null mutations; and step (ii) screening plants or grain obtained from the cross or mutagenesis, or progeny plants or grain obtained therefrom, by analysing DNA, RNA, protein, starch granules or starch from the plants or grain, and step (iii) selecting a fertile wheat plant that has reduced SSIIa activity relative to at least one of the parental wheat plants of step (i).

28. A process for screening wheat grain or a wheat plant, the method comprising determining the amount or activity of SSIIa relative to the amount or activity in wild-type wheat grain or a wild-type wheat plant and selecting grain, or a plant which produces grain, wherein the selected grain is the wheat grain according to claim 1, or the selected plant produces the wheat grain according to claim 1.

29. A process for producing a food comprising steps of (i) adding a food ingredient according to claim 22 to another food ingredient, and (ii) mixing the food ingredients, thereby producing the food.

30. (canceled)

31. A process for improving one or more parameters of metabolic health, bowel health or cardiovascular health in a subject in need thereof, or of preventing or reducing the severity or incidence of a metabolic disease such as diabetes, bowel disease or cardiovascular disease, the method comprising providing to the subject the food product of claim 24.

32. (canceled)

33. A process for producing starch, comprising the steps of i) obtaining wheat grain according to claim 1, and ii) extracting starch from the grain, thereby producing the starch.

34. A process of producing bins of wheat grain comprising:
a) reaping wheat stalks comprising the wheat grain of claim 1;
b) threshing and/or winnowing the stalks to separate the grain from chaff; and
c) sifting and/or sorting the grain separated in step b), and loading the sifted and/or sorted grain into bins, thereby producing bins of wheat grain.
